SEQUENCE LISTING (3 sequences)

SEQ ID NO: 1 - protein, 8 aa, artificial sequence
  synthetic peptide
SIINFEKL

SEQ ID NO: 2 - DNA, 1812 bp, artificial sequence
  synthetic polynucleotide (sequence encoding anti-murine FOLR2-Fc-antigen)
  CDS (7)..(1806)
gccaccatgggctggagctgcatcatcctgttcctggtggccaccgccaccggcgtgcacagcctcgaggacatcgtgatgacccagagccccagcagcctggccgtgagcgccggcgagaccgtgaccatcaactgcaagagcagccagagcctgctgtacagcggcaaccagaagaactacctggcctggtaccagcagaagcccggccagagccccaagctgctgatccactgggccagcaccaggcagagcggcgtgcccgacaggttcatcggcagcggcagcggcaccgacttcaccctgaccatcagcagcgtgcaggccgaggacctggccatctactactgccagcagtactacgacagccccctgaccttcggcagcggcaccaagctggagatcaagggcggcggcggcagcggcggcggcggcagcggcggcggcggcagcggcggcggcggcagcgaggtgcagctggtggagagcggcggcggcctggtgcagcccggcaggagcctgaagctgagctgcgccgccagcggcttcaccttcagcaactacgacatggcctgggtgaggcaggcccccaccaagtgcctggagtgggtggccagcatcagccccagcggcgacatcacctactacagggacagcgtgaagggcaggttcaccgtgagcagggacaacaccaagagcaccctgttcctgcagatggacagcctgaggagcgaggacaccgccacctactactgcgccaggcacggccccaccgaggcccccgacatcatggacgcctggggccagggcgccagcgtgaccgtgagcagcggcggcggcggcagcgccgccgccgcggccgctagatctgctcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacgccagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagcccgggcactggaaagcatcatcaacttcgagaagctgaccgaggccgccagcatctacagatactacggcctggccgctcccagcagctcctaccggagacctgtgggcatcagctctaacttcagctctctggagaacttcagagcctacgtggacggccagatcgccagcaacgagaacatggaaacaatggagtctgggcgcgccgactacaaagacgatgacgacaagtaagctagc

SEQ ID NO: 3 - protein, 599 aa, artificial sequence
  Synthetic Construct
MGWSCIILFLVATATGVHSLEDIVMTQSPSSLAVSAGETVTINCKSSQSLLYSGNQKNYLAWYQQKPGQSPKLLIHWASTRQSGVPDRFIGSGSGTDFTLTISSVQAEDLAIYYCQQYYDSPLTFGSGTKLEIKGGGGSGGGGSGGGGSGGGGSEVQLVESGGGLVQPGRSLKLSCAASGFTFSNYDMAWVRQAPTKCLEWVASISPSGDITYYRDSVKGRFTVSRDNTKSTLFLQMDSLRSEDTATYYCARHGPTEAPDIMDAWGQGASVTVSSGGGGSAAAAAARSAPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKARALESIINFEKLTEAASIYRYYGLAAPSSSYRRPVGISSNFSSLENFRAYVDGQIASNENMETMESGRADYKDDDDK

(19) United States
(12) Patent Application Publication
(10) Pub. No.: US 2024/0201192 A1
(43) Pub. Date: Jun. 20, 2024

(54) FOLR2+ MACROPHAGES AND ANTI-TUMOR IMMUNITY

(71) Applicants: Institut Curie, Paris (FR); Institut National de la Sante et de la Recherche Medicale, Paris (FR); Universite Paris Cite, Paris (FR); Centre National de la Recherche Scientifique, Paris (FR)

(72) Inventors: Rodrigo Nalio Ramos, Sao Paolo (BZ); Pierre Guermonprez, Paris (FR); Eliane Piaggio, Paris (FR); Julie Helft, Paris (FR)

(73) Assignees: Institut Curie, Paris (FR); Institut National de la Sante et de la Recherche Medicale, Paris (FR); Universite Paris Cite, Paris (FR); Centre National de la Recherche Scientifique, Patis (FR)

(21) Appl. No.: 18/286,252

(22) PCT Filed: Apr. 12, 2022
(86) PCT No.: PCT/EP2022/059792

§ 371 Date: Oct. 10, 2023

(30) Foreign Application Priority Data
Apr. 12, 2021 (EP) 21305475.2

(51) Int. Cl. G01N 33/574; C07K 16/28; C12Q 1/6886; G01N 33/569

(52) U.S. Cl. CPC G01N 33/57484; C07K 16/28; C12Q 1/6886; G01N 33/56972; C12Q 2600/118; C12Q 2600/158

(57) ABSTRACT

The invention relates to tumor-associated FOLR2+ macrophages and gene signature thereof as a biomarker of favorable outcome and anti-tumor immunity useful for the prognosis and monitoring of cancer patients. The invention relates also to FOLR2+ macrophages as a therapeutic target for enhancing T cell immunity in the prevention and treatment of cancer and infectious diseases.

### FIELD OF THE INVENTION

The invention pertains to the field of immunological biomarkers and immunotherapy. The invention relates to tumor-associated FOLR2+ macrophages and gene signature thereof as a biomarker of favorable outcome and anti-tumor immunity useful for the prognosis and monitoring of cancer patients. The invention relates also to FOLR2+ macrophages as a therapeutic target for enhancing T cell immunity in the prevention and treatment of cancer and infectious diseases.

### BACKGROUND OF THE INVENTION

Macrophage infiltration is a hallmark of solid cancers and overall macrophage infiltration is correlated with lower patient survival and resistance to therapy. For example, macrophages are one of the most abundant immune cell population in human breast tumors microenvironment (TME)(Cassetta and Pollard. 2018). Macrophage infiltration in breast tumor correlates with poor prognosis and higher tumor grades (Zhao et al., 2017)(Ruffell and Coussens, 2015)(Ramos et al., 2020). Tumor-associated macrophages (TAMs) play pro-tumoral roles by providing growth factors to tumors, enhancing tumor cell motility and invasion, and by promoting angiogenesis and metastasis (Lewis and Pollard, 2006)(Engblom et al., 2016)(Caux et al., 2016). In addition, TAMs exert immunosuppressive functions thereby preventing tumor cell destruction by NK and T lymphocytes. Therefore, targeting TAM recruitment, survival and function has become a major therapeutic goals (Ries et al., 2014)(Mantovani et al., 2017)(Binnewies et al., 2018).

Even though the current paradigm presents TAMs as pro-tumorigenic cells in most instances, several studies have highlighted protective roles for TAMs in specific disease stages or organs (Ruffell and Coussens, 2015). Accordingly, targeting TAMs in preclinical cancer models can have a detrimental impact on tumor progression and metastasis (Bonapace et al., 2014)(Cassetta and Pollard, 2018)(Hanna et al., 2015). As described earlier (Mantovani et al., 2004), distinct populations of macrophages with opposite pro- and anti-tumoral functions might co-exist within the same tumor (Mantovani et al., 2002)(Ali et al., 2016). Tumor-associated macrophages are phenotypically and functionally heterogeneous. Specific tumor-associated macrophage subsets might be endowed with antagonistic role on cancer progression and on the development of anti-tumor immunity. Therefore, establishing the extent of heterogeneity in the macrophage compartment is a pre-requisite for the rational design of macrophage-targeting therapies.

Macrophage heterogeneity might potentially arise from i) alternative activation states (Mantovani et al., 2017), ii) imprinting by tissue- or tumor-associated cues defining macrophage niches (Cassetta et al., 2019)(Guilliams and Scott, 2017); iii) distinct TAM cellular origins (adult monocyte versus embryonic progenitors)(Franklin et al., 2014)(Ginhoux et al., 2010)(Loyher et al., 2018)(Zhu et al., 2017) and iv) tumor-induced systemic modification of circulating monocytes (Gallina et al., 2006)(Veglia et al., 2018)(Cassetta et al., 2019)(Ramos et al., 2020).

In human breast cancer, macrophage infiltration has been assessed with markers like CD14 or CSF1R (Ruffell et al., 2012)(Cassetta et al., 2019) and CD68 (Leek et al., 1996)(Yuan et al., 2014). However, CD14 and CSF1R also mark undifferentiated monocytes while CD68 expression among phagocytes is not fully characterized. Other markers like CD163, TIE2, MRC1/CD206 or MARCO have been implemented to assess TAM phenotypic heterogeneity (Cassetta and Pollard, 2018). Pioneer single cell RNA sequencing (scRNAseq) studies have invalidated alternative activation as the main mechanism accounting for TAM heterogeneity (Azizi et al., 2018). In summary, the phenotypic and functional diversity of TAM infiltrating human breast cancer remain to be elucidated.

Therefore, reliable markers specific for distinct subsets of Tumor-associated macrophages (TAMs) reflecting the antagonistic roles of TAM subsets are missing for the prognosis and treatment of cancer.

The present invention fulfills this need.

### SUMMARY OF THE INVENTION

In order to understand the phenotypic and functional heterogeneity of macrophage populations in human breast cancers, the inventors have generated a single-cell atlas of myeloid cells infiltrating human luminal breast tumors. They have identified two phenotypically distinct TAM subsets: TREM2⁺ macrophages expressing Triggering Receptor Expressed by Myeloid cells-2 (TREM2), Osteopontin (SPP1) and Cell Adhesion Molecule 1 (CADM1) genes; and FOLR2⁺ macrophages expressing Folate Receptor 2 (FOLR2), selenoprotein P (SEPP1), solute carrier family 40 member 1 (SLC40A1), Hyaluronan receptor (LYVE-1) and Mannose Receptor C-Type 1 (MRC1/CD206) genes.

Here, the inventors show that FOLR2⁺ macrophages are tissue-resident macrophages (TRMs) evolutionarily conserved across species and populating healthy mammary glands prior the onset of cancer development.

In stark contrast with TREM2⁺ macrophage whose infiltration has been previously associated to worst clinical outcomes (Molgora et al., 2020), the inventors show that the abundance of FOLR2⁺ TAMs is predictive of better clinical outcomes. Specific gene signatures defining FOLR2⁺ macrophages are an independent prognostic factor which positively correlates with patient survival in breast cancer and across at least six other types of cancer.

Accordingly, FOLR2⁺ macrophages positively correlate with signatures of major cellular players of anti-tumor immunity, including CD8⁺ T cells, NK cells and dendritic cells (DCs).

It was further shown that FOLR2+ and TREM2+ macrophages are spatially segregated within the tumor microenvironment (TME). FOLR2+ macrophages specifically locate in peritumoral, stromal areas, including perivascular regions. Moreover, FOLR2⁺ TAMs co-localize with lymphoid aggregates containing CD8+ T cells in breast cancer and across ten other types of cancers. This FOLR2⁺ macrophage/CD8⁺ T cell co-localization correlates with favorable clinical outcomes suggesting an anti-tumorigenic role for this newly characterized macrophage subset.

In addition, the inventors have shown that FOLR2+ macrophages have a higher capacity to activate T cells than TREM2+ (CADM1+) macrophages. Furthermore, vaccination using Anti-FOLR2 targeting antibody coupled to a model antigen elicit a specific CD8+ T cell response.

This study highlights antagonistic roles for tumor-associated macrophage subsets and paves the way for subset-specific therapeutic interventions in macrophages-based cancer therapies. Based on these results, the inventors propose to use FOLR2+ tumor-associated macrophages as biomarker for the prognosis and monitoring of cancer and to target antigens to FOLR2-expressing macrophages to enhance T cell immunity for preventing or treating cancer and infectious diseases.

The present invention relates to a method of prognosis and monitoring of cancer in a patient, comprising:
determining the level of FOLR2+ macrophages in a patient tumor sample, wherein the level of tumor-associated FOLR2+ macrophages correlates positively with outcome of cancer disease or treatment in the patient, and
deducing therefrom whether the outcome of cancer disease or treatment is likely to be favorable or unfavorable in the patient.

In some embodiments of the method, an elevated level of FOLR2+ macrophages in the patient tumor sample as compared to a reference, indicates that the outcome of cancer disease or treatment is likely to be favorable in the patient.

In some embodiments of the method, the favorable outcome of cancer disease comprises an increased survival time or rate, a decreased rate of relapse, an increased time to relapse, and/or a reduced tumor evolution or metastasis.

In some embodiments, the method comprises determining the density of FOLR2+ cells in the patient tumor sample; preferably by immunohistochemical technique using anti-FOLR2 antibody; preferably wherein the FOLR2+ cells are further TREM2− or TREM2^{low} and/or CADM1−.

In some other embodiments, the method comprises determining the level of expression of FOLR2 gene in the patient tumor sample; preferably comprising determining the level of FOLR2 protein.

In some other embodiments, the method comprises determining the level of expression of a gene signature of tumor-associated FOLR2+ macrophages, which comprises or consists of the FOLR2, SEPP1 and SLC40A1 genes; preferably comprising determining the levels of mRNA expressed by said genes.

In some preferred embodiments, the method comprises determining the level(s) of mRNA expressed by the FOLR2 gene or the FOLR2, SEPP1 and SLC40A1 genes by RNA-Seq.

In some embodiments, the method further comprises a step of classification of the patient into favorable and unfavorable prognosis groups based on the level of tumor-associated FOLR2+ macrophages determined in the patient tumor sample.

The invention also relates to a gene signature of tumor-associated FOLR2+ macrophages comprising or consisting of the FOLR2, SEPP1 and SLC40A1 genes and its in vitro use, as a biomarker for the prognosis or monitoring of cancer in a patient.

In some embodiments of the above method and use according to the invention, the cancer is selected from the group consisting of: breast, kidney, lung, liver, skin, uterus and adrenal gland cancer; preferably breast cancer; or the cancer is selected from the group consisting of: breast, kidney, lung, liver, skin, uterus, brain, thyroid and adrenal gland cancer; preferably breast cancer.

The invention also relates to a targeted antigen delivery system comprising a FOLR2 binding ligand associated with an antigen of interest or a nucleic acid encoding the antigen in expressible form.

In some embodiments of the targeted antigen delivery system, the antigen of interest is a vaccine antigen, preferably selected from tumor antigens and antigens from pathogens, in particular viral, bacterial, fungal, and parasite antigens.

In some embodiments of the targeted antigen delivery system, the FOLR2 binding ligand comprises an anti-FOLR2 antibody or fragment thereof comprising the antigen-binding site.

In some embodiments of the targeted antigen delivery system, the FOLR2 binding ligand and antigen or nucleic acid thereof are associated in a conjugate, a fusion protein or a particle; preferably wherein the particle is selected from the group consisting of lipoparticle, nanoparticle, virus-like particle, viral vector particle and combination thereof; more preferably wherein the particle incorporates the antigen or nucleic acid thereof and presents the FOLR2 binding ligand at its surface.

The present invention also relates to a pharmaceutical composition, comprising the antigen delivery system according to the present disclosure, and at least one pharmaceutically acceptable vehicle, adjuvant and/or carrier, and its use for stimulating T cell immune response specific for the antigen in the prevention and treatment or cancer and infectious diseases.

### DETAILED DESCRIPTION OF THE INVENTION

The invention provides the abundance of tumor-associated FOLR2-positive (FOLR2+) macrophages as a biomarker of favorable outcome and anti-tumor immunity in cancer patients. The invention further provides gene signatures defining FOLR2+ macrophages useful for measuring the biomarker. The invention provides the various uses of the biomarker and gene signatures for the prognosis and monitoring of cancer. The invention also provides, antigen-delivery systems targeting FOLR2+ macrophages and their use for stimulating T cell immune response in the prevention and treatment of cancer and infectious diseases.

### Definitions

Macrophages are a type of leukocyte of the immune system which are mononuclear phagocytes. Macrophages play a critical role in innate and adaptative immunity, as well as in tissue-homeostasis. Macrophages differentiate from embryonic precursors or from circulating monocytes and remain in different tissues including tumors. Macrophages residing in healthy tissues are named Tissue-resident macrophages (TRM). Macrophages infiltrating tumors are named Tumor-associated macrophages or TAM. Macrophages may be defined by various combination of markers as disclosed in the present examples. These include in particular: CD3E−, CD19/20−, CD66−, XCR1−, CD1C−, CCR2−, CD64+, CD11c+, HLA-DR+, CD14+ and APOE+; APOE+, APOC1+, C1QA+ and C1QB+.

FOLR2⁺ macrophages refer to a distinct subset of Tumor-associated macrophages or TAM. FOLR2+ macrophages differ from other subsets of TAMs such as TREM2⁺ macrophages by the differential expression of specific genes (gene signature) as shown in the examples and figures of the present application.

As used herein, «gene signature», «gene expression signature», «molecular signature» refers to a single or combined group of genes in a cell with a uniquely characteristic pattern of gene expression that occurs as a result of an altered or unaltered biological process or pathogenic medical condition.

As used herein, “biomarker” refers to a distinctive biological or biologically derived indicator of a process, event or condition. Biomarker includes “molecular marker” which refers to a specific gene or gene product (mRNA or protein).

As used herein, antitumor immunity refers to immune responses mediated by immune cells present in the tumor environment including in particular CD8⁺ T cells, NK cell, B cells. These cells may be organized in inflammation-induced lymphoid structures called tertiary lymphoid structures.

As used herein, antigen refers to any substance that can be specifically recognized by the immune system and in particular by the antibodies and the cells of the immune system (B lymphocytes, CD4+ T lymphocytes, CD8+ T lymphocytes). The antigen according to the invention refers to an immunogenic substance able to induce a specific immune response, such as the production of antibodies, and/or the induction of a T-helper response (activation of CD4+ T lymphocytes) and/or cytotoxic T response (activation of CD8+ T lymphocytes) specific for said antigen.

As used herein, the term “cancer” refers to any member of a class of diseases or disorders characterized by uncontrolled division of cells and the ability of these cells to invade other tissues, either by direct growth into adjacent tissue through invasion or by implantation into distant sites by metastasis. Metastasis is defined as the stage in which cancer cells are transported through the bloodstream or lymphatic system. The term cancer according to the present invention also comprises cancer metastases and relapse of cancer. Cancers are classified by the type of cell that the tumor resembles and, therefore, the tissue presumed to be the origin of the tumor. For example, carcinomas are malignant tumors derived from epithelial cells. This group represents the most common cancers, including the common forms of breast, prostate, lung, and colon cancer. Lymphomas and leukemias include malignant tumors derived from blood and bone marrow cells. Sarcomas are malignant tumors derived from connective tissue or mesenchymal cells. Mesotheliomas are tumors derived from the mesothelial cells lining the peritoneum and the pleura. Gliomas are tumors derived from glia, the most common type of brain cell. Germinomas are tumors derived from germ cells, normally found in the testicle and ovary. Choriocarcinomas are malignant tumors derived from the placenta. As used herein, “cancer” refers to any cancer type including solid and liquid tumors.

As used herein infectious diseases refers to any disease caused by a pathogenic agent or microorganism such as virus, bacteria, fungi, parasite and the like.

As used herein, the term “subject” refers to both human and non-human animal, in particular a mammal, such as with no limitations a rodent, a feline, a canine, a bovine, an ovine, an equine and a primate. A “patient” refers to a subject affected by a disease. Preferably, a subject or patient according to the invention is a human. The patient is preferably a cancer patient.

As used herein, the term “tumor sample” of a patient refers to any biological sample comprising cancer cells of said patient. For example, the tumor sample may be a sample from a primary tumor, metastasis, and/or tumor-draining lymph nodes (TDLN). Preferably, it is a tumor biopsy.

Samples include direct samples and processed samples. Processed samples have been treated by standard methods, used to prepare a biological sample for analysis. In particular, processed samples include samples that have been treated by standard methods used for the preparation of tissue for immunohistological analysis, or the isolation and purification of nucleic acids or proteins for analysis, such as those described in the Examples.

The term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies. As used herein, the terms “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and include suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment.

“Treating cancer” includes, without limitation, reducing the number of cancer cells or the size of a tumor in the patient, reducing progression of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms, slowing the progression of symptoms and others.

As used herein, “drug” or “therapeutic agent” refers to a compound or agent that provides a desired biological or pharmacological effect when administered to a human or animal, particularly results in an intended therapeutic effect or response on the body to treat or prevent conditions or diseases. Therapeutic agents include any suitable biologically-active chemical compound or biologically derived component.

As used herein, a “therapeutic response” or “response to treatment with a drug” refers to a positive medical response characterized by objective parameters or criteria such as objective clinical signs of the disease, patient self-reported parameters and/or the increase of survival. The objective criteria for evaluating the response to drug-treatment will vary from one disease to another and can be determined easily by one skilled in the art by using clinical scores. A positive medical response to a drug can be readily verified in appropriate animal models of the disease which are well-known in the art.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

### Prognosis and Monitoring of Cancer

The invention provides a biomarker and derived molecular diagnostic test useful for predicting the outcome of cancer disease and treatment in a patient.

The present invention shows that the abundance or level of tumor-associated FOLR2+ macrophages correlates positively with cancer outcome and anti-tumor immunity. Therefore, the level of tumor-associated FOLR2+ macrophages is a biomarker for the prognosis of cancer useful to predict the outcome of cancer disease in a patient before undergoing cancer treatment or in the course of cancer treatment. Furthermore, antitumor immunity is a predictive factor for cancer treatment efficacy. Therefore, it is considered that the level of tumor-associated FOLR2+ macrophages is also a biomarker for monitoring cancer treatment useful to predict the response to treatment, in particular a treatment comprising immunotherapy, such as checkpoint blockade therapies, in a cancer patient.

The invention provides a method of prognosis and monitoring of cancer in a patient, comprising measuring the level of FOLR2+ macrophages in a patient tumor sample, wherein the level of tumor-associated FOLR2+ macrophages correlates positively with outcome of cancer disease or treatment in the patient. This means, that the higher the level of tumor-associated FOLR2+ macrophages in the patient sample, the more favorable the outcome of cancer disease and treatment is likely to be in the patient. Therefore, according to the method of the invention, an elevated level of FOLR2+ macrophages in a patient tumor sample indicates that the outcome of cancer disease or treatment is likely to be favorable in the patient.

In some embodiments, the method according to the invention comprises:
determining the level of FOLR2+ macrophages in a patient tumor sample, wherein the level of tumor-associated FOLR2+ macrophages correlates positively with outcome of cancer disease or treatment in the patient, and
deducing therefrom whether the outcome of cancer disease or treatment is likely to be favorable or not in the patient.

A favorable outcome of cancer disease may comprise one or more of: an increased survival time or rate, a decreased rate of relapse; an increased time to relapse; a reduced tumor evolution or metastasis. A favorable outcome of cancer treatment means a positive medical response characterized by objective parameters or criteria such as a reduction of tumor growth, reduction of tumor marker expression, and others that are well-known in the art.

The level of FOLR2+ macrophages in the patient tumor sample (e.g. the level of tumor-associated FOLR2+ macrophages) may be determined directly, by measuring the level of FOLR2 positive (FOLR2+) cells in the patient tumor sample, or indirectly, by measuring the level of expression of the FOLR2 gene in the patient tumor sample, alone or in combination with other genes specific for FOLR2+ macrophages, and forming a gene signature of tumor-associated FOLR2+ macrophages.

According to the method of the invention, the presence of an elevated level of FOLR2+ macrophages in a patient tumor sample may be determined by comparison with a reference. The reference may be a reference sample comprising known levels of FOLR2+ cells; FOLR2 mRNA or protein; mRNA or protein expressed by signature genes. Alternatively, the reference is a predetermined value. The predetermined value may be a threshold value or a range. A reference value refers to a value established by statistical analysis of values obtained from representative panels of individuals. The reference value may for example be obtained by measuring FOLR2+ macrophage levels as disclosed above, in samples from a panel of cancer patients with favorable prognosis (for example, increased survival) and a panel of cancer patients with unfavorable prognosis (for example, no increased survival), as disclosed in the present examples. A cut-off value that can discriminate favorable and unfavorable prognosis of cancer is then determined. The panel of cancer patients is preferably of the same type of cancer as the tested patient.

In some embodiments, the level of tumor-associated FOLR2+ macrophages is determined directly, by measuring the level of FOLR2 positive (FOLR2+) cells in the patient tumor sample. The level of FOLR2+ cells in the patient tumor sample may be measured by immunohistological technique using anti-FOLR2 antibody, according to well-known methods such as disclosed in the present examples. The method may comprise determining the density of FOLR2+ cells in the patient tumor sample, which means the number of FOLR2+ cells per surface unit of tumor sample, wherein the unit maybe square millimetre (mm²). In some preferred embodiments, the FOLR2 positive (FOLR2+) cells are further TREM2 negative or “low” (TREM2− or TREM2^{low}) and/or CADM1 negative (CADM1−).

In some other embodiments, the level of tumor-associated FOLR2+ macrophages is determined indirectly, by measuring the level of expression of FOLR2 gene in the patient tumor sample. The method may comprise measuring the level of mRNA or protein expressed by the FOLR2 gene. Preferably, the method comprises measuring the level of FOLR2 protein.

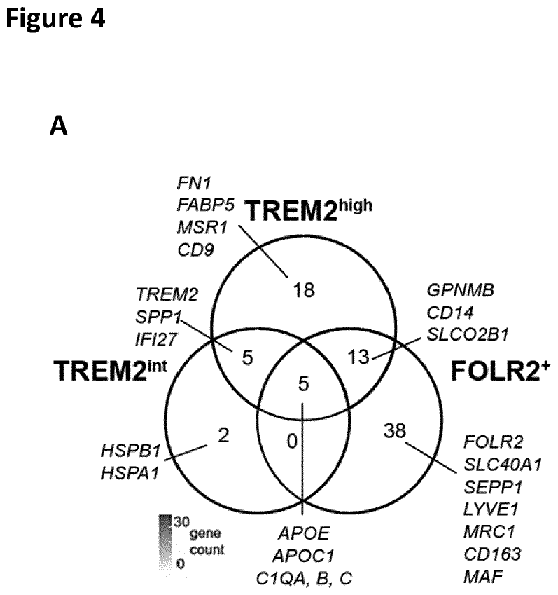

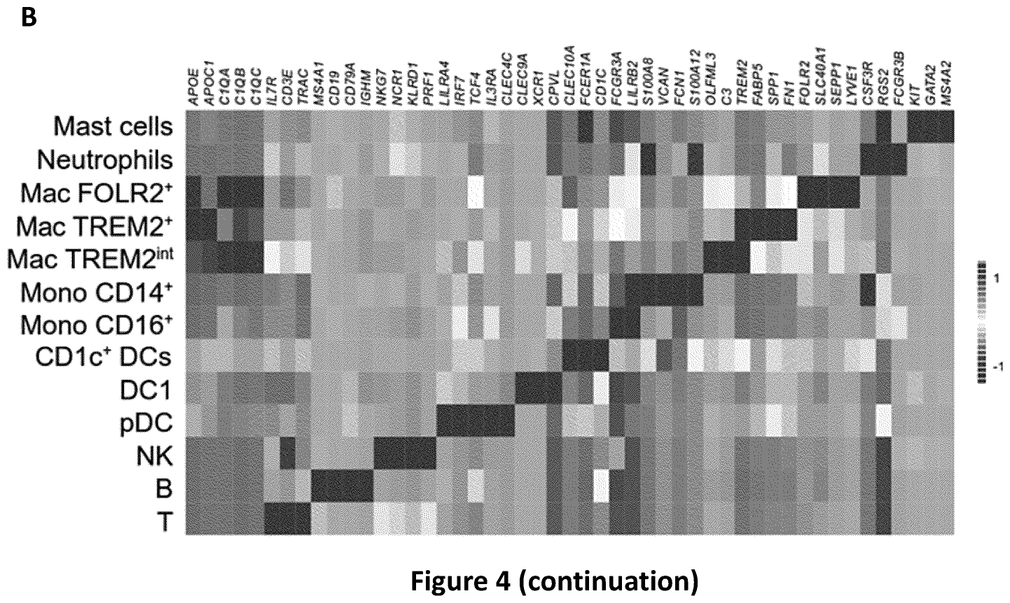

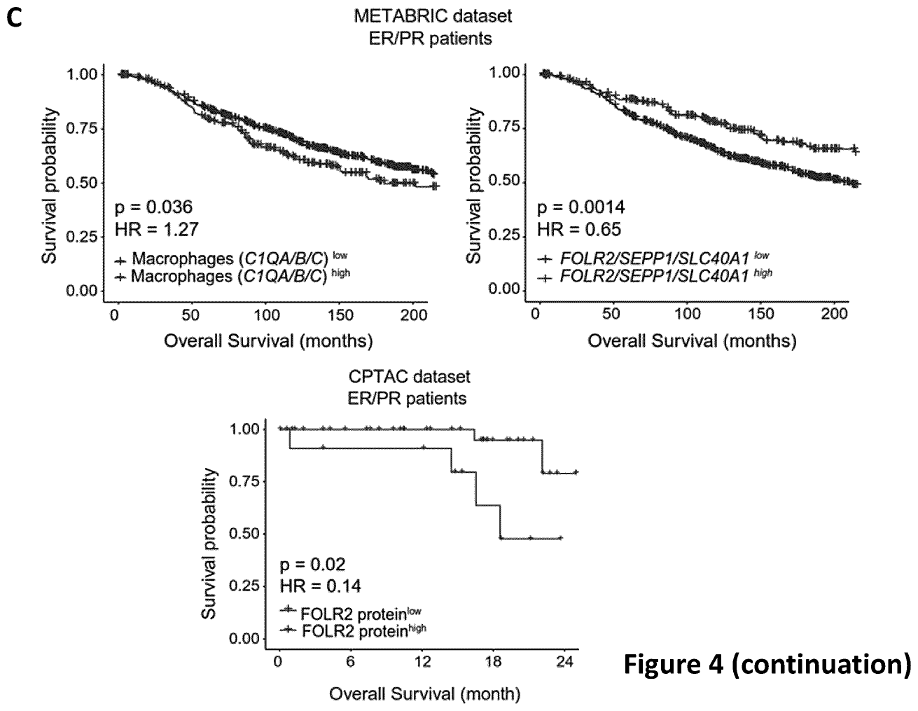

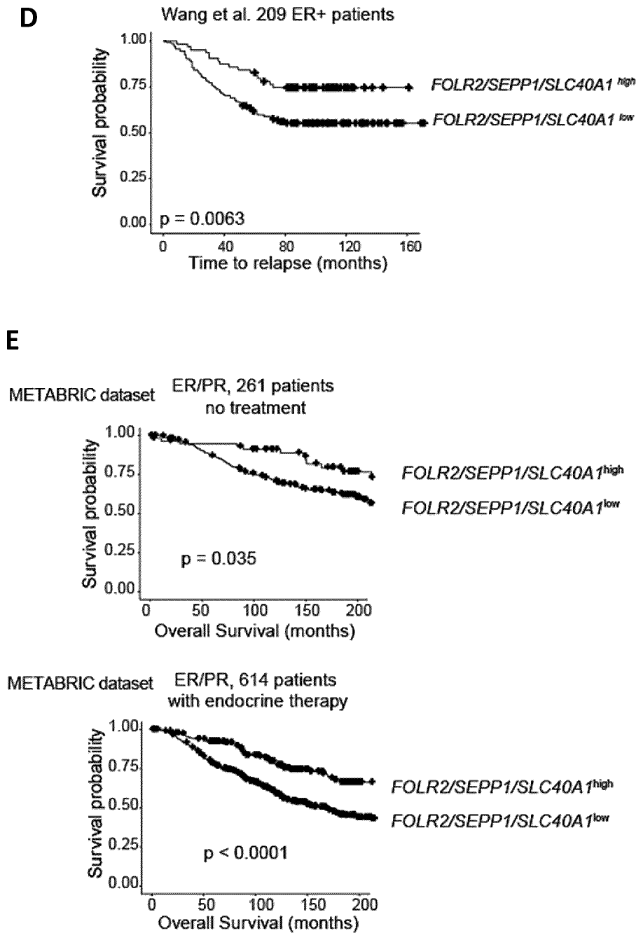

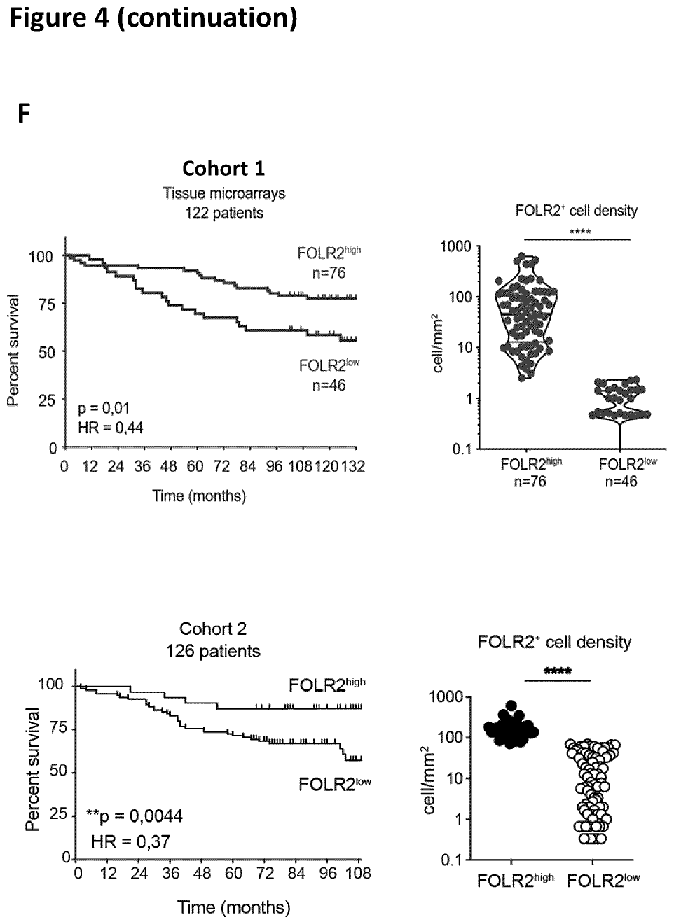

In some preferred embodiments, the level of tumor-associated FOLR2+ macrophages is determined indirectly, by measuring the level of expression of a gene signature of tumor-associated FOLR2+ macrophages, which comprises or consists of the FOLR2, SEPP1 and SLC40A1 genes. The cut-off value to stratify patients with high or low expression of the signature is determined by calculating all cut-off possible and choosing the cut-off with the best p-value. Alternatively, cut-off can be determined as the 25% of patients within a cohort with the highest expression of the signature, as compared to the 75% of patients with a lower expression of the same signature. As shown in the examples (FIG. 4A-B), this gene signature is specific for FOLR2+ macrophages and allows to differentiate FOLR2+ macrophages from other TAMs and other leukocytes lineages. Preferably, the method comprises measuring the levels of mRNA expressed by the signature genes.

In some embodiments, TAM-FOLR2⁺ macrophages express at least Folate Receptor 2 (FOLR2), selenoprotein P (SEPP1) and solute carrier family 40 member 1 (SLC40A1) genes and do not express Osteopontin (SPP1) and Cell Adhesion Molecule 1 (CADM1) genes, i.e., the TAM-FOLR2+ macrophages gene signature comprises FOLR2. SEPP1 and SLC40A1 and does not comprise SPP1 and CADM1. In some embodiments, TAM-FOLR2⁺ macrophages further express one or more or all of Hyaluronan receptor (LYVE-1), Mannose Receptor C-Type 1 (MRC1/CD206), CD163 and MAF genes, i.e., the TAM-FOLR2+ macrophages gene signature comprises one or more or all of LYVE-1, MRC1/CD206, CD163 and MAF. In some embodiments, TAM-FOLR2⁺ macrophages further express low level of Triggering Receptor Expressed by Myeloid cells-2 (TREM2), i.e., the TAM-FOLR2+ macrophages gene signature is TREM2-low. In some embodiments, FOLR2⁺ macrophages further do not express Triggering Receptor Expressed by Myeloid cells-2 (TREM2), i.e., the TAM-FOLR2+ macrophages gene signature does not comprise TREM2. In some embodiments, TAM-FOLR2⁺ macrophages further do not express one or more or all of FN1, FABP5, MSR1, CD9, IFI27, HSPB1 and HSPA1 genes, i.e., the TAM-FOLR2+ macrophages gene signature does not comprise one or more or all of FN1, FABP5, MSR1, CD9, IFI27, HSPB1 and HSPA1. In some particular embodiments, the TAM-FOLR2+ macrophages gene signature does not comprise SPP1, C3 and CD9.

As used herein, the term “gene expression level” or “level of expression of a gene” refers to an amount or a concentration of a transcription product (or transcript), for instance mRNA, or of a translation product, for instance a protein or polypeptide. Typically, a level of mRNA expression can be expressed in units such as transcripts per cell or nanograms per microgram of tissue. A level of a polypeptide can be expressed as nanograms per microgram of tissue, for example. Alternatively, relative units can be employed to describe a gene expression level.

As used herein, the expression of “measuring the level of expression of a gene” encompasses the step of measuring the quantity of a transcription product, preferably mRNA obtained through transcription of said gene, and/or the step of measuring the quantity of translation product, preferably the protein obtained through translation of said gene.

Typically, gene expression levels may be determined according to the routine techniques, well-known of the person skilled in the art. For example, the measurement may comprise contacting the patient tumor sample with selective reagents such as probes, primers, ligands or antibodies, and thereby detecting the presence of nucleic acids or proteins of interest originally in the sample.

Methods for determining the quantity of mRNA are well known in the art. For example, the mRNA contained in the sample is first extracted according to standard methods, for example using lytic enzymes or chemical solutions or extracted by nucleic-acid-binding resins following the manufacturer's instructions. The extracted mRNA present in the sample is then detected by any suitable method such as with no limitations: spectrophotometric methods; Hybridization such as Northern Blotting, Microarray, in situ hybridization such as RNAscope; Sequencing such as next generation sequencing (NGS) and Single-molecule sequencing; micro and nanosensor-based electrochemical, electrical, mechanical or optical detection and Nucleic acid amplification techniques. Nucleic acid amplification methods include isothermal and polymerase chain reaction (PCR)-based techniques such as for example, reverse transcription-PCR (RT-PCR), quantitative PCR (Q-PCR) in particular real time Q-PCR, RT-qPCR, droplet digital PCR (ddPCR), PCR-HM (High Resolution DNA Melting. PCR coupled to ligase detection reaction based on fluorescent microspheres (Luminex® microspheres).

In some embodiments, mRNA present in the sample is detected by nucleic acid amplification, nucleic acid hybridization or nucleic acid sequencing assay or a combination thereof. mRNA may be amplified using any suitable nucleic acid amplification technique such as described above or combinations thereof. Nucleic acid amplification assay uses at least one oligonucleotide primer specific for the mRNA, usually a pair of forward primer (sense primer) and reverse primer (anti-sense) specific for the mRNA, and preferably also an oligonucleotide probe specific for the mRNA for detecting any amplified product. The mRNA is subjected to a reverse transcription reaction with a reverse primer before amplification. Preferably, the amplification is reverse transcription polymerase chain reaction (RT-PCR), more preferably real-time reverse transcription polymerase chain reaction (RT-qPCR). The amplification or hybridization products are detected using suitable label for nucleic acid that are well-known in the art and include, fluorescent, chemiluminescent, radioactive, enzymatic labels or other.

RNA-Seq (RNA-sequencing) also called whole transcriptome shotgun sequencing (WTSS) is a technique that can examine the quantity and sequences of RNA in a sample using next generation sequencing (NGS) (Review in Wang et al., Nat. Rev. Genet., 2009, 10, 57-63). It analyzes the transcriptome of gene expression patterns encoded within RNA. RNA-seq has been adapted to single-cell analysis and single-cell RNAseq was first reported by Tang et al. (Nat. Methods, 2009, 6, 377-382); review in Wang et al., Nature Reviews Genetics, 2009, 10, 57-63 and Svensson et al. (Nat Protoc. 2018 April; 13(4):599-604).

In a preferred embodiment, the mRNA expression level is measured by RNA-Seq.

The level of the protein may be determined by any suitable methods known by skilled persons. Usually, these methods comprise contacting a cell sample, preferably a cell lysate, with a binding partner capable of selectively interacting with the protein present in the sample. The binding partner is generally a polyclonal or monoclonal antibody, preferably monoclonal. The methods generally include suitable labels such as fluorescent, chemiluminescent, radioactive, enzymatic labels or dye molecules, or other methods for detecting the amount of complex formed between the protein and the antibody or antibodies reacted therewith. The quantity of the protein may be measured, for example, by semi-quantitative Western blots, enzyme-labelled and mediated immunoassays, such as ELISAs, biotin/avidin type assays, radioimmunoassay, immune-electrophoresis, mass spectrometry, immunoprecipitation or by protein or antibody arrays.

The folate receptor beta (FOLR2) gene (also known as BETA-HFR, FBP, FBP/PL-1, FOLR1, FR-BETA, FR-P3, FRbeta) encodes a member of the folate receptor (FOLR) family, which have a high affinity for folic acid and for several reduced folic acid derivatives, and mediate delivery of 5-methyltetrahydrofolate to the interior of cells. The gene is expressed in placenta and hematopoietic cells. Expression is increased in malignant tissues. Human FOLR2 corresponds to the Gene ID: 2350. Human FOLR2 protein corresponds to the amino acid sequence UniProtKB/Swiss-Prot accession number P14207. Four transcript variants that encode the same protein have been found for this gene (GenBank/NCBI accession number: NM_000803.5 (variant 1); NM_001113534.2 (variant 2); NM_001113535.2 (variant 3); NM_001113536.2 (variant 4), all accessed on Mar. 22, 2021).

The triggering receptor expressed on myeloid cells 2 (TREM2) gene (also known as PLOSL2, TREM-2, Trem2a, Trem2b, Trem2c) encodes a membrane protein that forms a receptor signaling complex with the TYRO protein tyrosine kinase binding protein. The encoded protein functions in immune response and may be involved in chronic inflammation by triggering the production of constitutive inflammatory cytokines. Alternative splicing results in multiple transcript variants encoding different isoforms. TREM2 is broadly expressed in brain, lung and 14 other tissues. Human TREM2 corresponds to the Gene ID: 54209. Human TREM2 protein corresponds to the amino acid sequence UniProtKB/Swiss-Prot accession number Q9NZC2. Two transcript variants that encode the same protein have been found for this gene (GenBank/NCBI accession number: NM_018965.4 (variant 1); NM_001271821.2 (variant 2); all accessed on Mar. 22, 2021).

The cell adhesion molecule 1 (CADM1) gene (also known as BL2; ST17; IGSF4; NECL2; RA175; TSLC1; IGSF4A; Necl-2; SYNCAM; sgIGSF; sTSLC-1; synCAM1) encodes a cell adhesion molecule broadly expressed in lung, thyroid and 23 other tissues. Human CADM1 corresponds to the Gene ID: 23705. Human CADM1 protein corresponds to the amino acid sequence UniProtKB/Swiss-Prot accession number Q9BY67. Five transcript variants that encode the same protein have been found for this gene (GenBank/NCBI accession number: NM_014333.4 (variant 1); NM_001098517.2 (variant 2); NM_001301043.2 (variant 3); NM_001301044.2 (variant 4); NM_001301045.2 (variant 5); all accessed on Mar. 29, 2021).

The selenoprotein P (SEPP1) gene (also known as SeP, SELP, SEPP, SELENOP) encodes a selenoprotein that is predominantly expressed in the liver and secreted into the plasma. This selenoprotein is unique in that it contains multiple selenocysteine (Sec) residues per polypeptide (10 in human), and accounts for most of the selenium in plasma. It has been implicated as an extracellular antioxidant, and in the transport of selenium to extra-hepatic tissues via apolipoprotein E receptor-2 (apoER2). Human SEPP1 gene corresponds to Gene ID: 6414. Human selenoprotein P corresponds to the amino acid sequence UniProtKB/Swiss-Prot accession number P49908. Three transcript variants that encode the same protein have been found for this gene (GenBank/NCBI accession number NM_005410.4 (variant 1); NM_001085486.3 (variant 2); NM_001093726.3 (variant 3) all accessed on Mar. 22, 2021).

The solute carrier family 40 member 1 (SLC40A1) gene (also known as FPN1, HFE4, MTP1, IREG1, MST079, MSTP079, SLC11A3) encodes a cell membrane protein that may be involved in iron export from duodenal epithelial cells. Defects in this gene are a cause of hemochromatosis type 4 (HFE4). The gene is expressed in placenta, intestine, muscle and spleen; it is also detected in erythrocytes (at protein level). Human SLC40A1 gene corresponds to the Gene ID: 30061. Human SLC40A1 protein corresponds to the amino acid sequence UniProtKB/Swiss-Prot accession number Q9NP59. The transcript corresponds to the nucleotide sequence GenBank/NCBI accession number NM_014585.6 acessed on Feb. 20, 2021.

Various antibodies directed specifically to the FOLR2, TREM2, CADM1, SEPP1, or SLC40A1 protein have been disclosed and are publicly available; see in particular the antibodies used in the present examples or other antibodies disclosed on antibody related databases or portals such as with no limitations: antibodypedia, Antibody Group (ABG), Antibody Central, The hybridoma Databank, European Collection of Cell Cultures; Monoclonal Antibody Index, SCOP, Validated antibody database (VAD); Antibody Chemically Defined (ABCD) data base.

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarcinoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Kaposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pincaloma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Kaposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), car, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilms tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

In some embodiments, the cancer is selected from the group consisting of: breast, kidney, lung, liver, skin, uterus and adrenal gland cancer. In some embodiments, the cancer is selected from the group consisting of: breast, kidney, lung, liver, skin, uterus, brain, thyroid and adrenal gland cancer. Kidney cancer includes Kidney Renal Cell Carcinoma (KIRC); Lung cancer includes Lung adenocarcinoma (LUAD); Liver cancer includes Liver hepatocellular carcinoma (LIHC); uterus cancer includes cervical cancer, in particular cervical squamous cell carcinoma and endocervical adenocarcinoma (CESC); skin cancer includes melanoma (SKCM); adrenal gland cancer includes adrenocortical carcinoma (ACC); breast cancer includes estrogen receptor positive (ER+), progesterone positive (PR+), HER2 positive (HER2+) and triple-negative (ER−, PR−, HER2−) breast cancer. “Triple-negative breast cancer” refers to any breast cancer that does not overexpress the genes for estrogen receptor (ER), progesterone receptor (PR) and HER2/Neu. This subtype of breast cancer is clinically characterized as more aggressive and less responsive to standard treatment and associated with poorer overall patient survival. Breast cancer includes in particular luminal cancer (ER/PR+; HER2−). Brain cancer includes glioma such as Brain Lower Grade Glioma (LGG). Thyroid cancer includes Thyroid carcinoma (THCA). In particular embodiments, the cancer is selected from the group comprising: luminal breast cancer, Kidney Renal Cell Carcinoma (KIRC), Lung adenocarcinoma (LUAD), Liver hepatocellular carcinoma (LIHC), cervical squamous cell carcinoma, endocervical adenocarcinoma (CESC), melanoma (SKCM), glioma such as Brain Lower Grade Glioma (LGG) and Thyroid carcinoma (THCA); particularly selected from the group comprising: luminal breast cancer, Kidney Renal Cell Carcinoma (KIRC), Lung adenocarcinoma (LUAD), Liver hepatocellular carcinoma (LIHC), cervical squamous cell carcinoma, endocervical adenocarcinoma (CESC) and melanoma (SKCM). In some preferred embodiments, the cancer is breast cancer, in particular luminal breast cancer.

The method of the invention is useful to establish the prognosis of cancer at the early stage of the disease and thereby adapt cancer treatment in the patient depending on the initial prognosis determined on untreated patient. The method of the invention is also useful for monitoring cancer during the course of treatment and adjusting cancer treatment depending on the prognosis determined on treated patient.

The method of the invention is also useful to predict response to cancer therapy, in particular immunotherapy such as checkpoint blockade therapy. Patients with higher levels of FOLR2+ macrophages are likely to be good responders to immunotherapy such as checkpoint blockade therapy as they have elevated levels of immune cell infiltration in the tumor or tumor environment. Therefore, patients with higher levels of FOLR2+ macrophages, will be treated with immunotherapy, in particular checkpoint blockade therapy.

Interestingly, breast cancer patients, in particular ER/PR+ patients with high FOLR2 gene-signature have improved survival from endocrine therapy compared to chemotherapy alone, or chemotherapy plus endocrine therapy which is not the case in FOLR2 gene-signature low patients. FOLR2 high patients would benefit from endocrine therapy alone and do not require chemotherapy. While FOLR2 low patients could benefit from combined endocrine and chemotherapy. In some advantageous embodiments, the method further comprises a step of classification of the patient(s) into favorable and unfavorable prognosis groups based on the level of tumor-associated FOLR2+ macrophages determined in the patient(s) tumor sample.

Another aspect of the invention relates a composition for use in a method of treating a cancer patient, wherein the composition is administered to a patient previously diagnosed as having a favorable prognosis according to the method of prognosis according to the invention. In some embodiments, the composition comprises an immunotherapeutic agent, in particular an immune checkpoint blockage agent. In some embodiments, the composition comprises an endocrine therapy agent, in particular for treating a breast cancer patient.

Another aspect of the invention relates a composition for use in a method of treating a cancer patient, wherein the composition is administered to a patient previously diagnosed as having an unfavorable prognosis according to the method of prognosis according to the invention.

The composition may comprise an agent for immunotherapy or chemotherapy or a combination thereof. In some embodiments, the composition comprises a combination of at least an endocrine therapy and a chemotherapy agent, in particular for treating a breast cancer patient.

Patients diagnosed as having a good prognosis using the method of prognosis according to the invention may benefit from a less aggressive cancer treatment, in terms of both treatment type and treatment regimen; thereby reducing side-effects and improving patient's comfort and well-being. On the opposite, patient diagnosed as having a poor prognosis using the method of prognosis according to the invention, may benefit from a more aggressive cancer treatment, in terms of both treatment type and treatment regimen; thereby increasing the efficacy of treatment.

The invention also relates to a method of treating cancer, comprising: determining the level of FOLR2+ macrophages in a patient tumor sample according to the method of prognosis or monitoring of cancer according to the present disclosure; and administering an appropriate treatment to the patient depending on whether the outcome of cancer disease or treatment is likely to be favorable or not in the patient. In some embodiments, the method comprises the administration of an immunotherapeutic agent, in particular an immune checkpoint blockage agent if the patient is diagnosed as having a favorable prognosis. In some embodiments, the method comprises the administration of an endocrine therapy agent if the patient is diagnosed as having a favorable prognosis; preferably wherein the patient is a breast cancer patient as disclosed herein. In some other embodiments, the method comprises the administration of a chemotherapy agent, or a combination of chemotherapy agent and immunotherapy agent, if the patient is diagnosed as having an unfavorable prognosis. In some other embodiments, the method comprises the administration of a chemotherapy agent, or a combination of chemotherapy agent and endocrine therapy agent, if the patient is diagnosed as having an unfavorable prognosis; preferably wherein the patient is a breast cancer patient as disclosed herein.

Yet another aspect of the invention relates to the in vitro use of tumor-associated FOLR2+ macrophages, gene signature thereof, and FOLR2 gene, as favorable prognostic biomarker of outcome of cancer disease and treatment in patient. FOLR2 gene and gene signature of FOLR2+ macrophages include gene products (mRNA, protein). Preferably, the gene signature of FOLR2+ macrophages comprises or consists of the FOLR2, SEPP1 and SLC40A1 genes. The biomarker is used to determine the level of tumor-associated FOLR2+ macrophages in a patient tumor sample. Preferably, the biomarker is used to determine: (i) the density of tumor-associated FOLR2+ cells, preferably FOLR2+ and TREM2− and/or CADM1− cells; (ii) the level of FOLR2 mRNA or protein in the tumor sample, preferably the level of FOLR2 protein; (iii) the level of mRNA or protein expressed by a gene signature of FOLR2+ macrophages comprising or consisting of the FOLR2, SEPP1 and SLC40A1 genes; preferably the mRNA levels expressed by the signature genes.

The invention also to a gene signature of FOLR2+ macrophages comprising or consisting of the FOLR2, SEPP1 and SLC40A1 genes. The invention also relates to the in vitro use of the gene signature of FOLR2+ macrophages as a biomarker for the prognosis or monitoring of cancer.

### Therapy

The invention also provides antigen-delivery systems targeting FOLR2+ macrophages and their use for stimulating T cell immune response in the prevention and treatment of cancer and infectious diseases.

One aspect of the invention relates to a targeted antigen delivery system comprising a FOLR2 binding ligand associated with an antigen or a nucleic acid encoding the antigen in expressible form.

The antigen may be any antigen of interest, in particular a vaccine antigen. Vaccine antigens are well-known in the art and include tumor antigens and antigens from pathogens, such as viral, bacterial, fungal, parasite antigens and other antigens. The antigen may be a natural, recombinant or synthetic antigen, including complete antigens; antigen fragments or portions; and antigen constructs, in particular derived from several antigens. The antigen is specific for the tumor or pathogen; it may comprise one or more epitopes, including B cell, CD4+ T cell and/or CD8+ T cell epitopes. Any known vaccine antigen is suitable for incorporation into the vaccine delivery system of the invention and the delivery system according to the invention may incorporate any of the known vaccine antigens. The targeted antigen delivery system may be used to stimulate CD4+ and/or CD8+ T cell immune response specific for the antigen, including effector T cell and cytotoxic T cell immune responses specific for the antigen.

The nucleic acid encoding the antigen may consist of recombinant, synthetic or semi-synthetic nucleic acid which is expressible in the individual's target cells or tissue. The nucleic acid may be DNA, RNA, mixed and may further be modified. Said nucleic acid construct may be a mammalian expression cassette, preferably human expression cassette, wherein the coding sequence is operably linked to appropriate regulatory sequence(s) for their expression in an individual's target cells or tissue(s), such as promoter, intron, enhancer, terminator, and others. The nucleic acid may be incorporated in a suitable vector for gene delivery into individual's target cells or tissue(s) that are well-known in the art and include: plasmid and viral vector such as with no limitations: adenovirus, lentivirus, Adeno-associated virus (AAV), poxvirus such as vaccinia virus, replication-defective alphavirus replicons and cytomegalovirus.

The FOLR2 binding ligand binds to cell-surface FOLR2, in particular cell-surface human FOLR2. This means that the FOLR2 binding ligand as sufficient affinity for FOLR2 extracellular domain to form a stable complex, under standard conditions.

In some embodiments, the FOLR2 binding ligand comprises or consists of an anti-FOLR2 antibody or a fragment thereof comprising the antigen-binding site.

As used herein, the term “antibody” refers to a protein that includes at least one antigen-binding region of immunoglobulin. The antigen binding region may comprise one or two variable domains, such as for example a VH domain and a VL domain or a single VHH or VNAR domain. The term “antibody” encompasses full length immunoglobulins of any isotype, functional fragments thereof comprising at least the antigen-binding region and derivatives thereof. Antigen-binding fragments of antibodies include for example Fv, scFv, Fab, Fab′, F(ab′)2, Fd, Fabc and sdAb (V_{H}H, V-NAR). Antibody derivatives include with no limitation polyspecific or multivalent antibodies, intrabodies and immunoconjugates. Intrabodies are antibodies that bind intracellularly to their antigen after being produced in the same cell (for a review see for example, Marschall AL, Dübel S and Böldicke T “Specific in vivo knockdown of protein function by intrabodies”, MAbs. 2015; 7(6):1010-35). The antibody may be glycosylated. The antibody is preferably non-functional for antibody-dependent cytotoxicity and complement-mediated cytotoxicity. The antibody may comprise mutations in the Fc domain that prevent binding to high affinity Fc-gamma receptor. Such muttaions that are well-known in the art include for example N297A. The antibody may be a chimeric antibody comprising a Fv or scFv from anti-FOLR2 monoclonal antibody and constant domain(s), in particular CH2 and CH3 from another antibody. Antibodies are prepared by standard methods that are well-known in the art such as hybridoma technology, selected lymphocyte antibody method (SLAM), transgenic animals, recombinant antibody libraries or synthetic production.

The antibody is directed to the extracellular domain of FOLR2. Various anti-FOLR2 antibodies are known in the art and publicly available; see in particular the antibodies used in the present examples or other antibodies disclosed on antibody related databases or portals such as with no limitations: antibodypedia, Antibody Group (ABG), Antibody Central, The hybridoma Databank, European Collection of Cell Cultures; Monoclonal Antibody Index, SCOP, Validated antibody database (VAD); Antibody Chemically Defined (ABCD) data base. For example, anti-FOLR2 antibodies are disclosed in US 2008/0260812; US 2014/0010756; WO 2012/033987. A disulfide-stabilized Fv anti-FOLR2 is disclosed in Nagai et al., Arthritis and Rheumatism, 2006, 54, 3126-3134.

The antigen and the FOLR2 binding ligand may be associated directly or indirectly. Direct association refers, in particular to conjugates and fusion proteins. Fusion protein may comprise from N-ter to C-ter: anti-FOLR2 Fv, antibody CH domain(s), for example CH2 and CH3, and the antigen fused to the C-terminal end of the fusion protein. The antigen may be a polyepitopic polypeptide.

Indirect association refers in particular to non-covalent complexes and particles. Non-covalent complexes may be formed for example using binding interaction partners such as strepatavidin/biotin. Particles refer to any particle capable of delivering a therapeutic agent into cells. Particles include lipoparticles, microparticles, nanoparticles, exosomes, virus-like-particles, viral vector particles and combination thereof such as lipid nanoparticles (LNP). Folate-modified liposomal complex are disclosed in Tie et al. (Signal transduction and targeted therapy, 2020, 5, 6). In some embodiments, the particle incorporates the antigen or nucleic acid thereof and presents the FOLR2 binding ligand at its surface, in particular FOLR2 antibody or fragment thereof.

The targeted antigen delivery system is advantageously used in the form of an immunogenic or vaccine composition comprising, as active substance the antigen, and at least one pharmaceutically acceptable vehicle, adjuvant and/or carrier.

The pharmaceutical composition is formulated for administration by a number of routes, including but not limited to oral, parenteral and local. The pharmaceutical vehicles are those appropriate to the planned route of administration, which are well known in the art.

The pharmaceutical composition comprises a therapeutically effective amount of antigen sufficient to stimulate a specific T cell immune response in the administered subject, in particular an antitumoral response or protective immune response against the pathogen.

The pharmaceutically effective dose depends upon the composition used, the route of administration, the type of mammal (human or animal) being treated, the physical characteristics of the specific mammal under consideration, concurrent medication, and other factors, that those skilled in the medical arts will recognize.

The pharmaceutical composition of the present invention is generally administered according to known procedures, at dosages and for periods of time effective to induce a beneficial effect in the individual. The administration may be by injection or by local administration. The injection may be subcutaneous, or intramuscular.

In some embodiments, the pharmaceutical composition comprises another active agent such as in particular an immunomodulatory agent, an anticancer, antiviral, antibacterial, antifungal or antiparasitic agent.

The pharmaceutical composition of the invention is advantageously used in combination with additional cancer therapies such as with no limitations: immunotherapy including immune checkpoint therapy and immune checkpoint inhibitor, co-stimulatory antibodies, CAR-T cell therapy, anticancer vaccine; chemotherapy and/or radiotherapy. The combined therapies may be separate, simultaneous, and/or sequential.

In some embodiments, the pharmaceutical composition is used for the treatment of humans.

The invention encompasses the targeted antigen-delivery system and pharmaceutical composition for use for stimulation T-cell immune response in the prevention and treatment of cancer and infectious diseases.

The practice of the present invention will employ, unless otherwise indicated, conventional techniques which are within the skill of the art. Such techniques are explained fully in the literature.

The invention will now be exemplified with the following examples, which are not limitative, with reference to the attached drawings in which:

### FIGURE LEGENDS

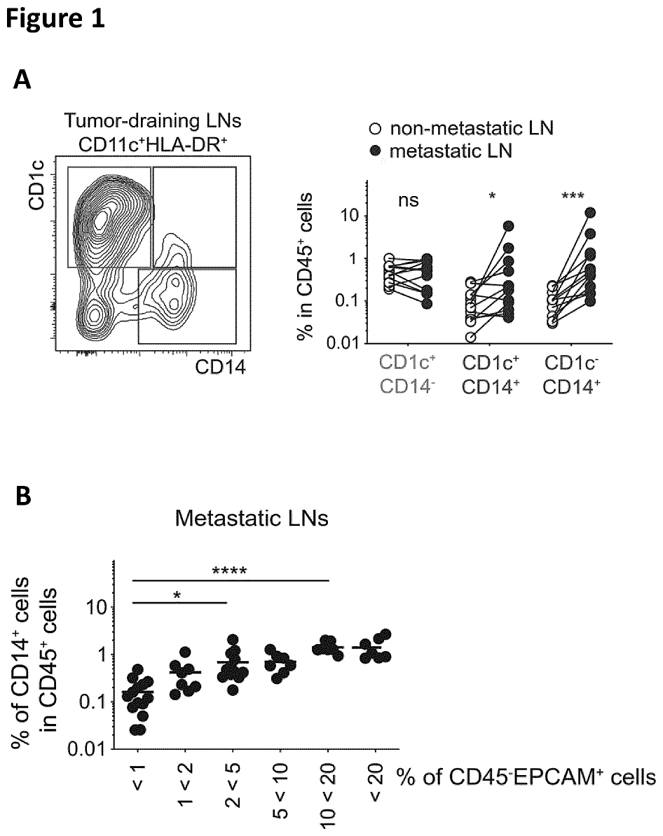

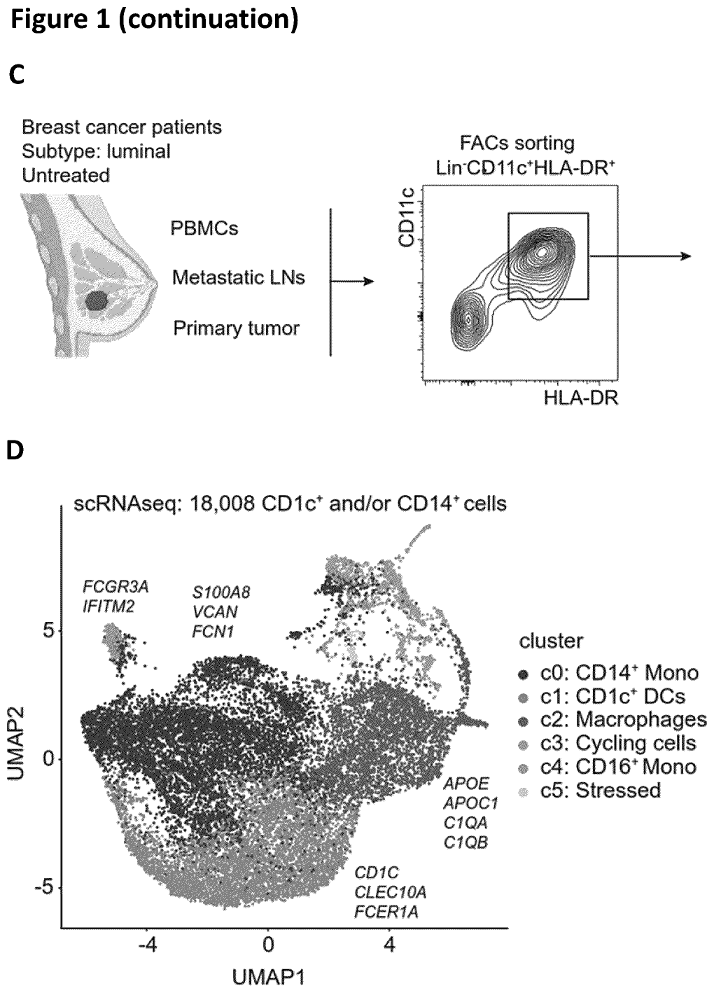

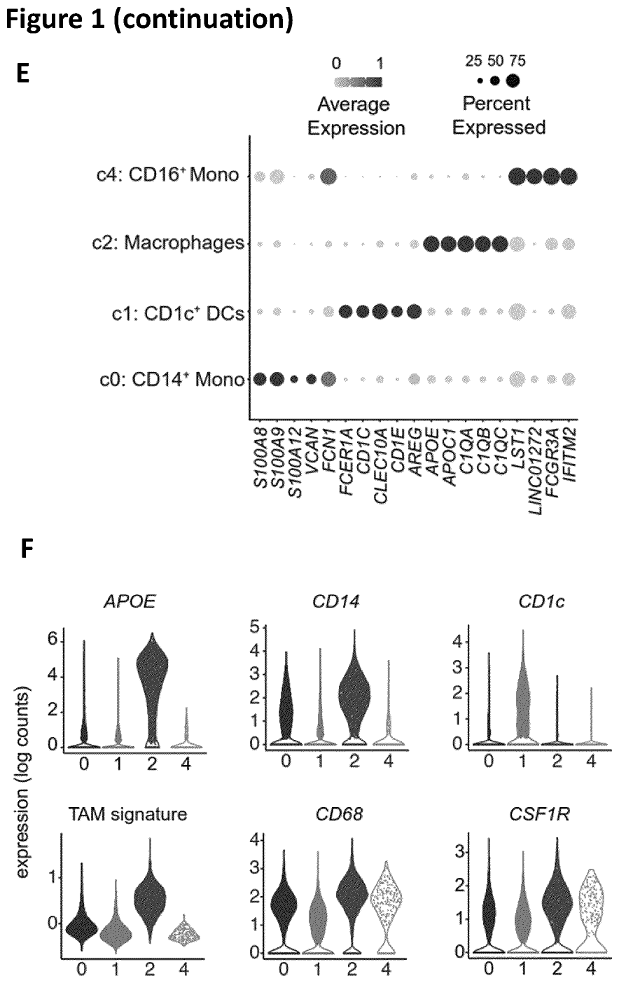

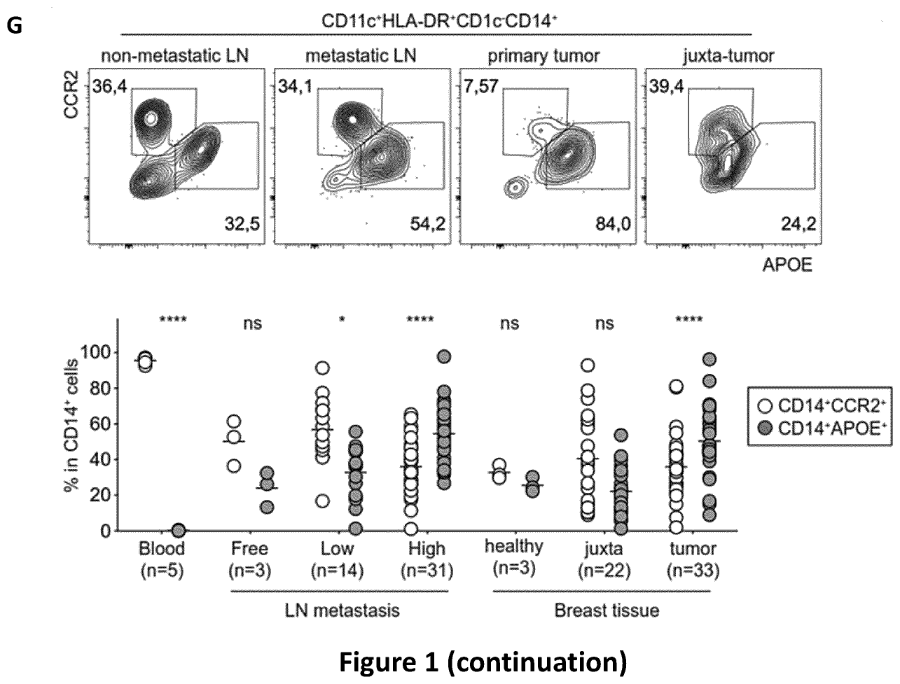

FIG. 1. APOE expression defines tumor-associated macrophages in human breast cancer.
A. Flow cytometry quantification of CD1c⁺CD14⁻, CD1c⁺CD14⁺ and CD1c⁻CD14³⁰ cells among CD11c⁺HLA-DR⁺ myeloid cells in matched non-metastatic and metastatic lymph nodes.
B. Flow cytometry quantification of CD14⁺ cells in stratified metastatic lymph nodes according to CD45⁻EPCAM⁺ tumor cells frequency.
C. Experimental design of untreated luminal breast cancer patient samples (PBMCs, metastatic lymph nodes and primary tumors) and gating strategy for FACS-sorting of myeloid cells for scRNAseq droplet-based assay (10× Chromium).
D. Dimensionality reduction of scRNAseq data merged from blood, metastatic lymph nodes and primary tumors was performed using a Louvain graph-based clustering identifying six clusters. Each dot represents an individual cell (n=18008). Representative genes from each cluster are depicted.
E. Bubble-map showing the top five most significant expressed genes across the six defined clusters. Circles sizes and represent percentage of cells within a cluster expressing a gene and color represents the average expression of each gene.
F. Violin plots illustrating expression distributions of selected genes of interest in clusters 0 (CD14⁺ monocytes), cluster 1 (CD1C⁺ DCs), cluster 2 (macrophages) and cluster 4 (CD16⁺ monocytes).
G. Flow cytometry quantification of CD14⁺CCR2⁺ monocytes versus CD14⁺APOE⁺ macrophages in healthy or metastatic lymph nodes and breasts. (*P≤0.05; ** P≤0.01; **** P<0.0001).

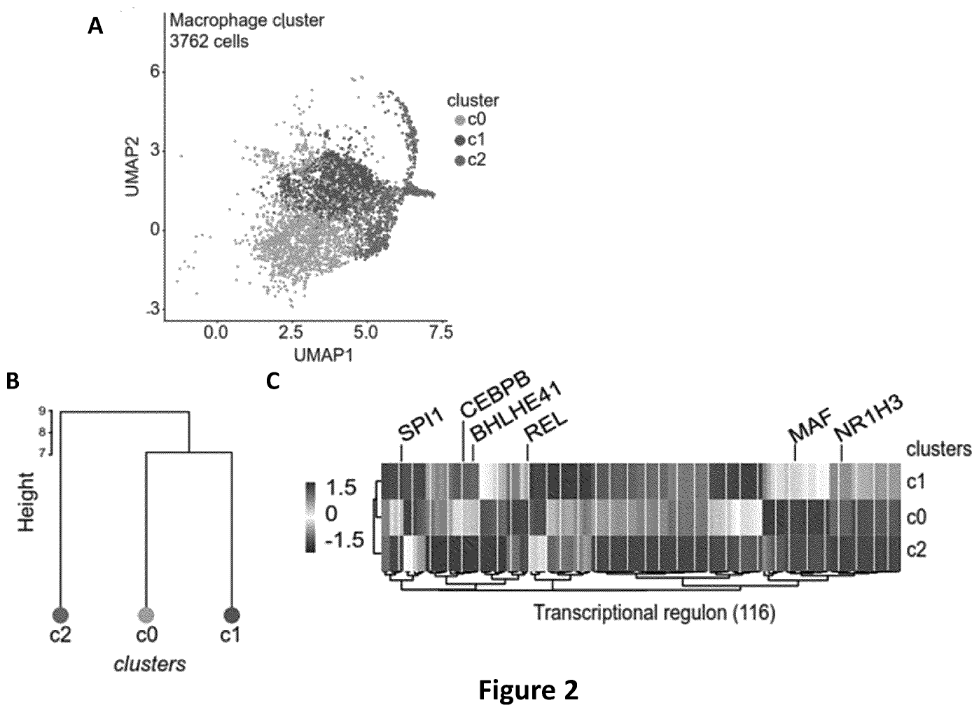

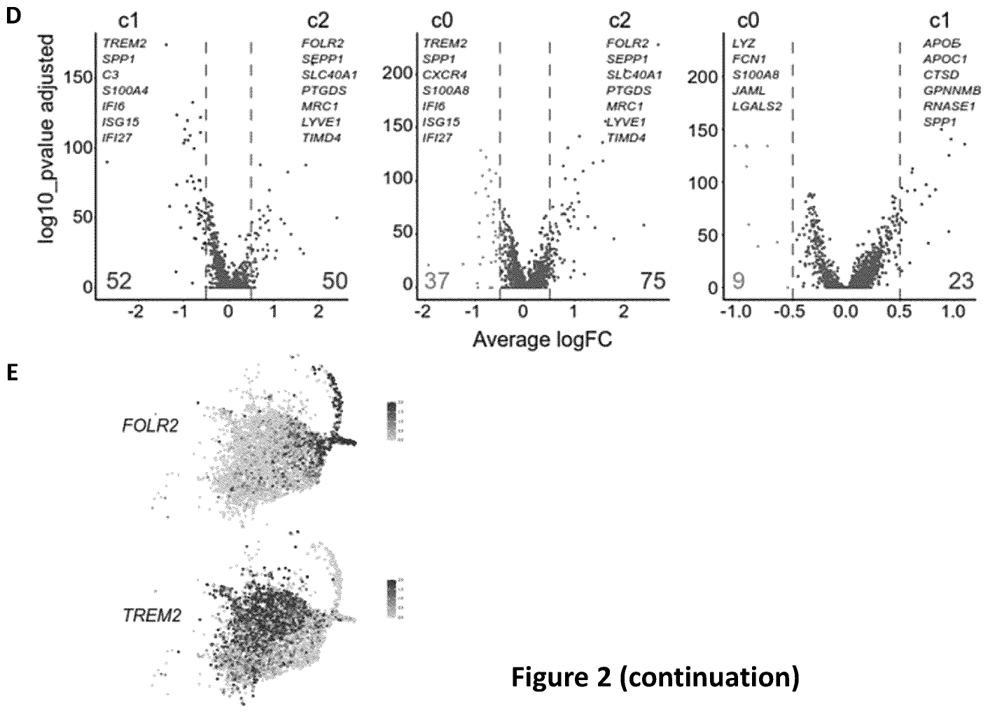

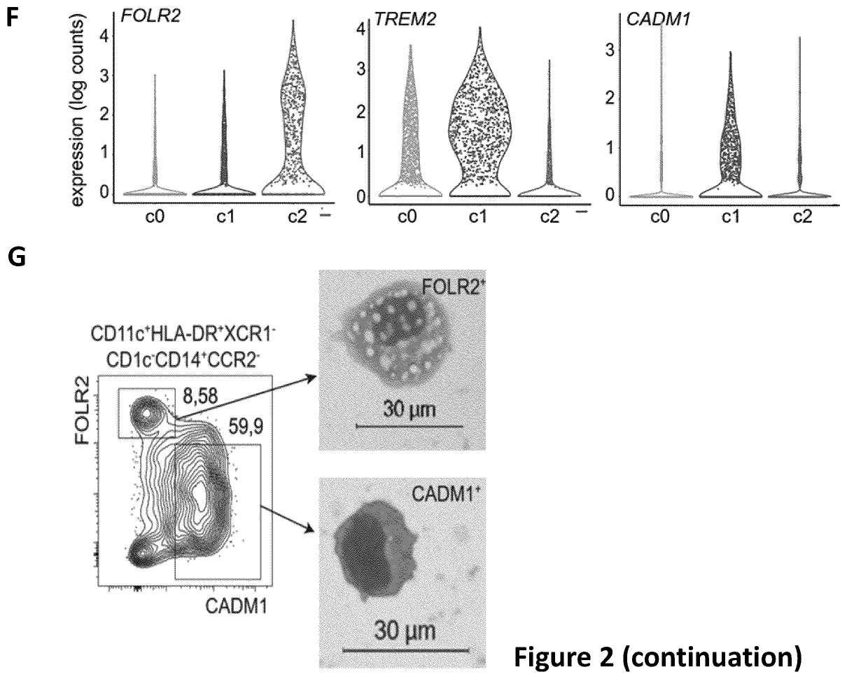

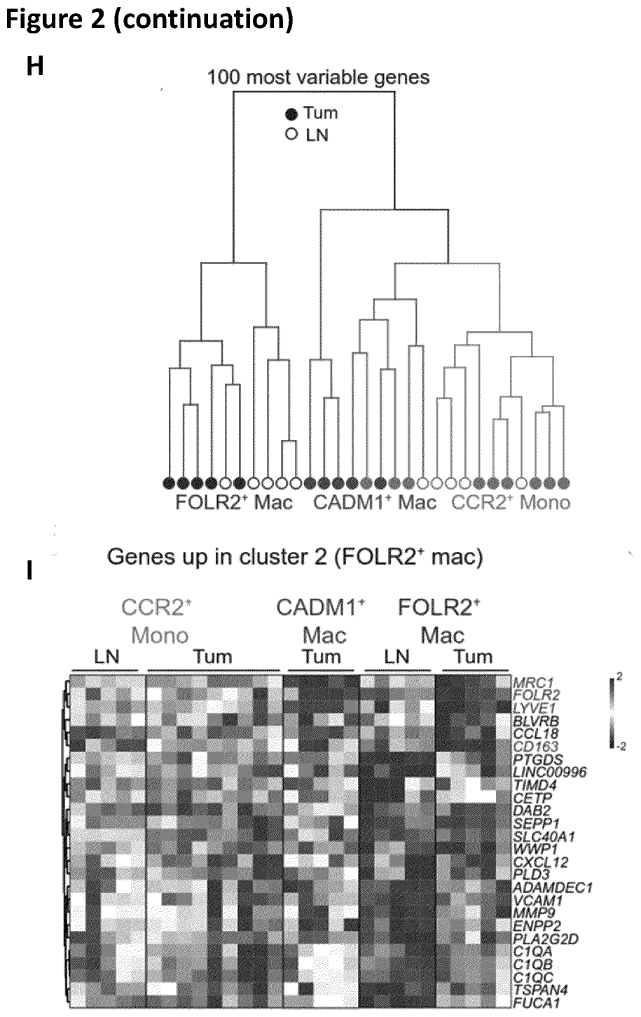

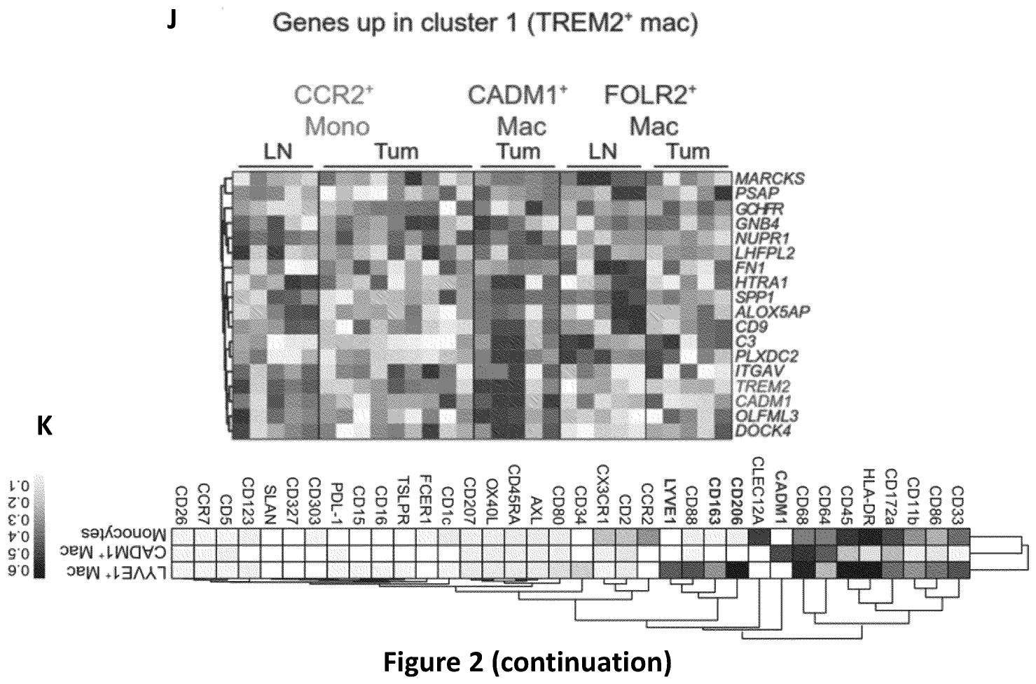

FIG. 2. Single cell RNA sequencing reveals two main subsets of APOE⁺ macrophages.
A. UMAP plot visualization of APOE⁺ macrophages (cluster 2 from FIG. 1D). Each dot represents an individual cell (n=3762).
B. Hierarchical clustering of clusters 0, 1 and 2 based on average gene expression (1200 genes).
C. Heatmap and hierarchical clustering of differently predicted transcriptional regulons in clusters 0, 1 and 2 defined using the SCENIC pipeline.
D. Volcano plot showing D.E.G. between each cluster. Selected genes among the Top25 were depicted.
E. UMAP plot showing mutually exclusive expression of TREM2 and FOLR2 in APOE⁺ macrophages.
F. Violin plots expression distributions of FOLR2, TREM2 and CADM1 across APOE⁺ macrophage clusters
G. Representative flow cytometry contour-plot and cytospin images from FACS-sorted FOLR2⁺ and CADM1⁺ macrophages isolated from metastatic lymph nodes and primary tumors.
H. Hierarchical clustering using the 100 most variable genes from bulk RNA sequencing of FOLR2⁺ TAMs, CADM1⁺ TAMs and CCR2⁺ monocytes isolated by FACS-sorting from metastatic lymph nodes and primary tumor of untreated luminal breast cancer patients.
I-J. Heatmap of the D.E.G. between FOLR2⁺ (c2) and TREM2^{high} (c1) macrophages selected from scRNAseq dataset and applied to bulk RNAseq of FOLR2⁺ macrophages, CADM1⁺ macrophages and CCR2⁺ monocytes isolated by FACS-sorting from metastatic lymph nodes and primary tumor of untreated luminal breast cancer patients.
K. Protein expression analysis (CyTOF) of monocyte/macrophage and dendritic cell markers in CD14⁺CCR2⁺ monocytes, CD14⁺CCR2⁻CD68⁺LYVE1⁺ macrophages and CD14⁺CCR2⁻CD68⁺CADM1⁺ macrophages from metastatic lymph nodes.

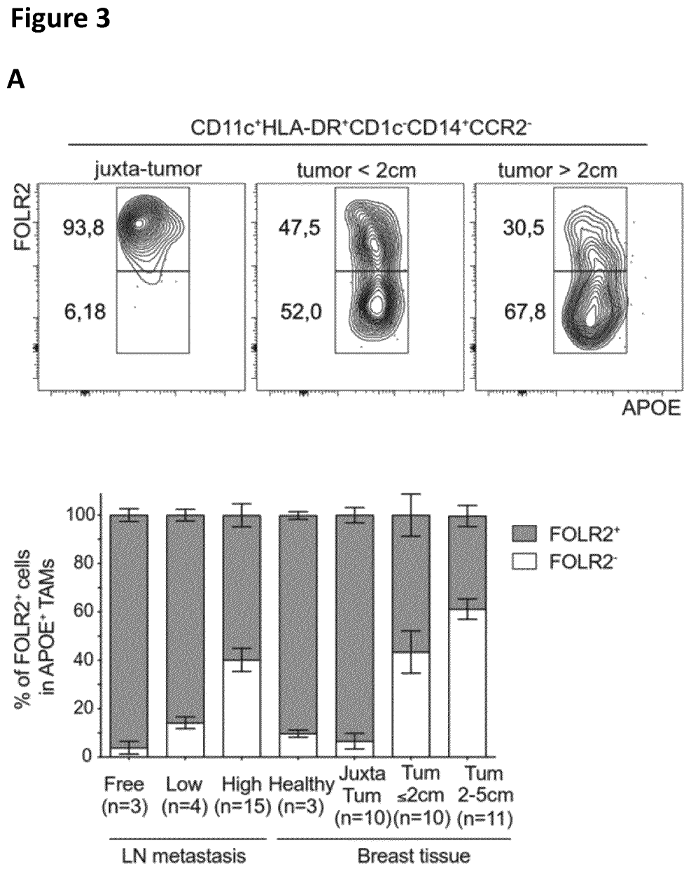

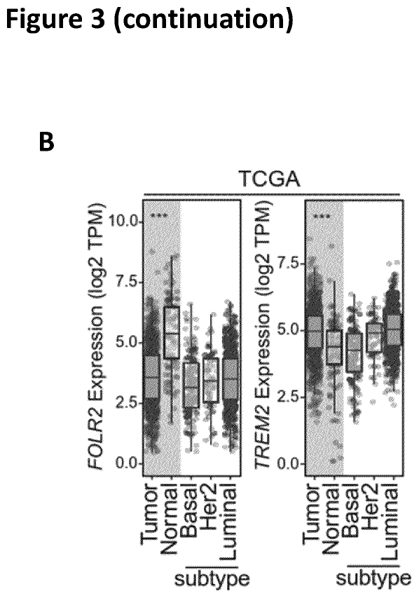

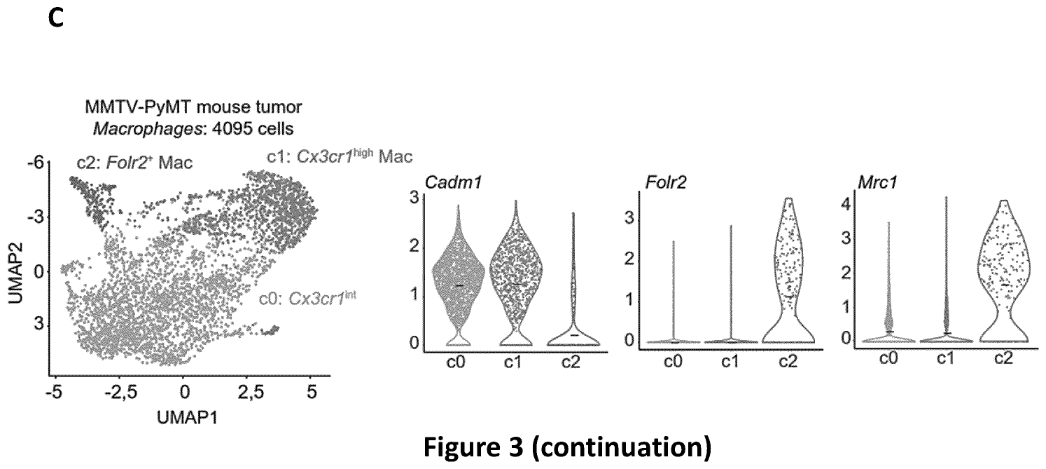

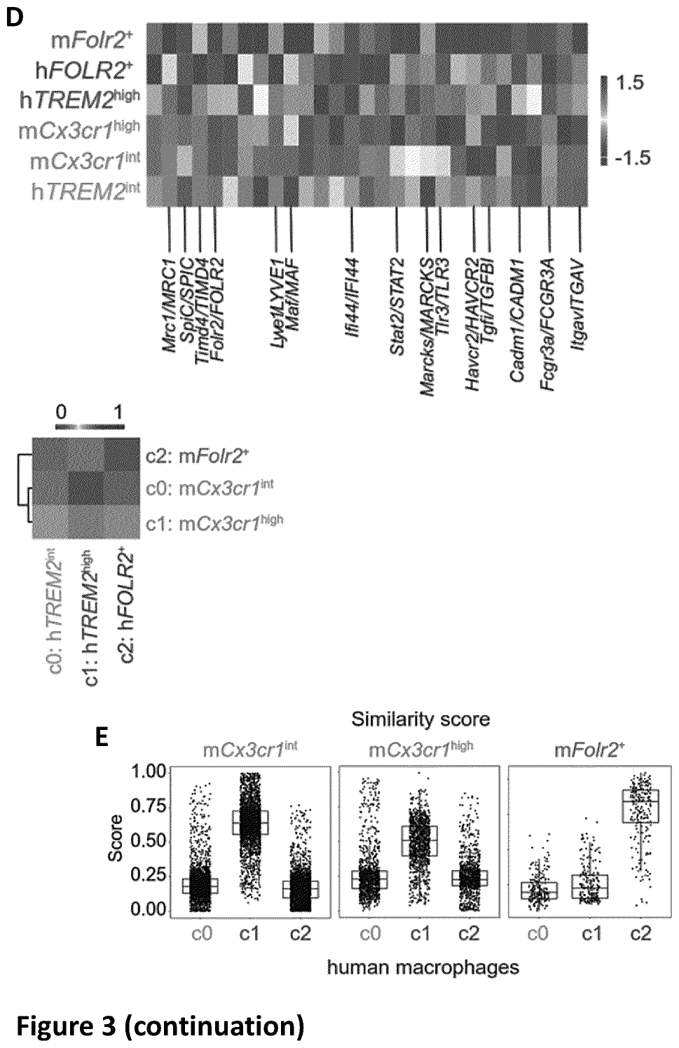

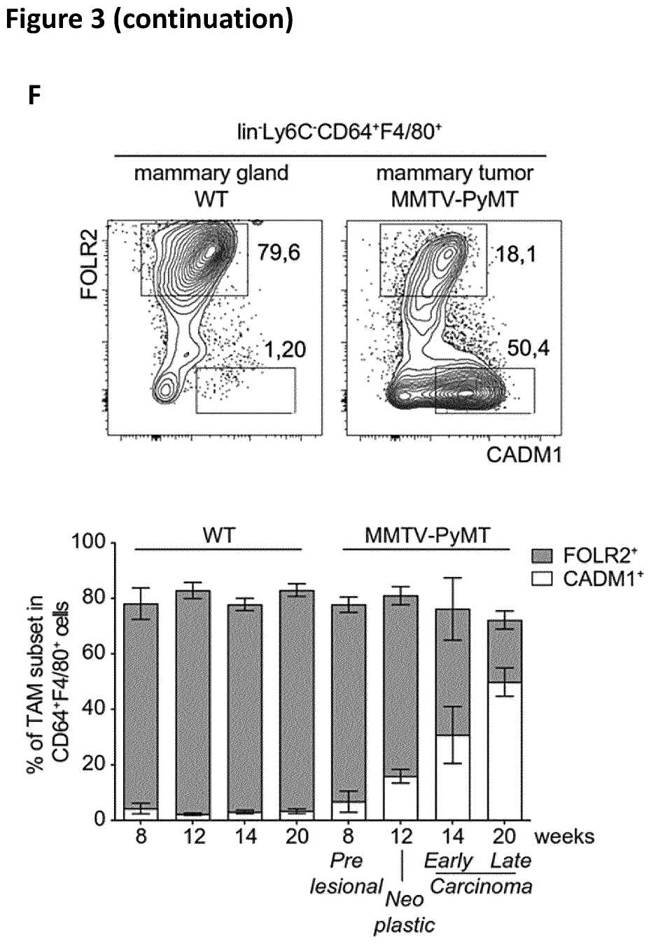

FIG. 3. FOLR2⁺ macrophages are tissue-resident macrophages.
A. Representative contour-plots (upper panel) and quantification (lower panel) of FOLR2⁺APOE⁺ and FOLR2⁻APOE⁺ macrophages across distinct breast cancer patient tissues using flow cytometry.
B. FOLR2 and TREM2 mRNA expression in tumor microarrays from breast cancer patients of the TCGA database (Tumor: n=1093; Normal breast: n=112; Basal subtype, n=190; Her2 subtype, n=82; and Luminal subtype, n=781).
C. UMAP plot visualization of Fcgr1⁺ macrophages (n=4095) isolated from mammary tumors of 23-week-old MMTV-PyMT mice (n=2). Violin plots illustrating expression distributions of Cadm1, Folr2 and Mrc1 across Fcgr1⁺ macrophages.
D. Heatmap showing gene orthologs similarly expressed across mouse and human macrophage subsets.
E. Similarity score defined using the Seurat v3 reference label transfer integration (Stuart et al., 2019). Graph plots showing prediction scores of each mouse macrophage cluster applied to each human macrophage cluster (defined in FIG. 2A). Each dot represents a cell. Heatmap of the mean of the prediction score.
F. Quantification of Folr2⁺ and Cadm1⁺ macrophages by flow cytometry during tumor development in MMTV-PyMT mouse model. Upper part shows representative contour-plots of macrophage subsets in mammary gland from tumor-free and tumor-bearing mice (20-week-old PyMT mouse). Lower part shows quantification of macrophage subsets in WT or PyMT mice during tumor development.

FIG. 4. FOLR2⁺ macrophages correlate with increased survival in breast cancer patients.
A. Venn diagram showing specific and common differentially expressed genes (DEG) of each APOE⁺ macrophages clusters (defined in FIG. 2A).
B. Heatmap showing mean expression of specific genes discriminating tumor-infiltrating immune cell populations in a published scRNAseq dataset performed on CD45³⁰ cells isolated from breast cancer patients (Azizi et al., 2018).
C. Kaplan-Meier survival curves generated for a macrophage gene signature (C1QA/C1QB/C1QC) and a FOLR2⁺ TAM gene signature (FOLR2/SEPP1/SLC40A1) in the METABRIC luminal breast cancer (BC) cohort (n=1309). Kaplan-Meier survival curve generated for FOLR2 protein expression in the CPTAC luminal breast cancer cohort (n=49). Patients were divided in high- and low-expressing groups based on 75% quantile of signature expression.
D. Kaplan-Meier survival curves generated for the FOLR2⁺ macrophage gene signature (FOLR2/SEPP1/SLC40A1) in the Wang et al. luminal breast cancer cohort (n=209 patients). Patients were divided in high- and low-expressing groups based on the 70% quantile of signature expression.
E. To test the prognostic power of the FOLR2 signature in distinct treatment groups, a univariate analysis was performed in luminal BC patients classified according to the therapy received. Kaplan-Meier survival curves generated for the FOLR2⁺ macrophage gene-signature (FOLR2/SEPP1/SLC40A1) in the METABRIC dataset in Luminal A/B patients stratified by treatment received. The FOLR2 gene-signature has a significant prognostic value in patients receiving endocrine therapy alone. The FOLR2 gene-signature did not have significant prognostic power in patients receiving endocrine therapy+chemotherapy or chemotherapy alone. Patients were divided in high- and low-expressing groups based on the 70% quantile of signature expression. Number of patients is indicated for each groups.
F. Kaplan-Meier survival curves generated for FOLR2⁺ macrophage density calculated by multispectral analysis of tumors (Cohort 1: n=122; Cohort 2: n=126). Graph shows the quantification of FOLR2⁺ macrophage density in tumors. Patients were divided in high- and low- cell-density groups based on best p value cut-off (**P≤0.01).

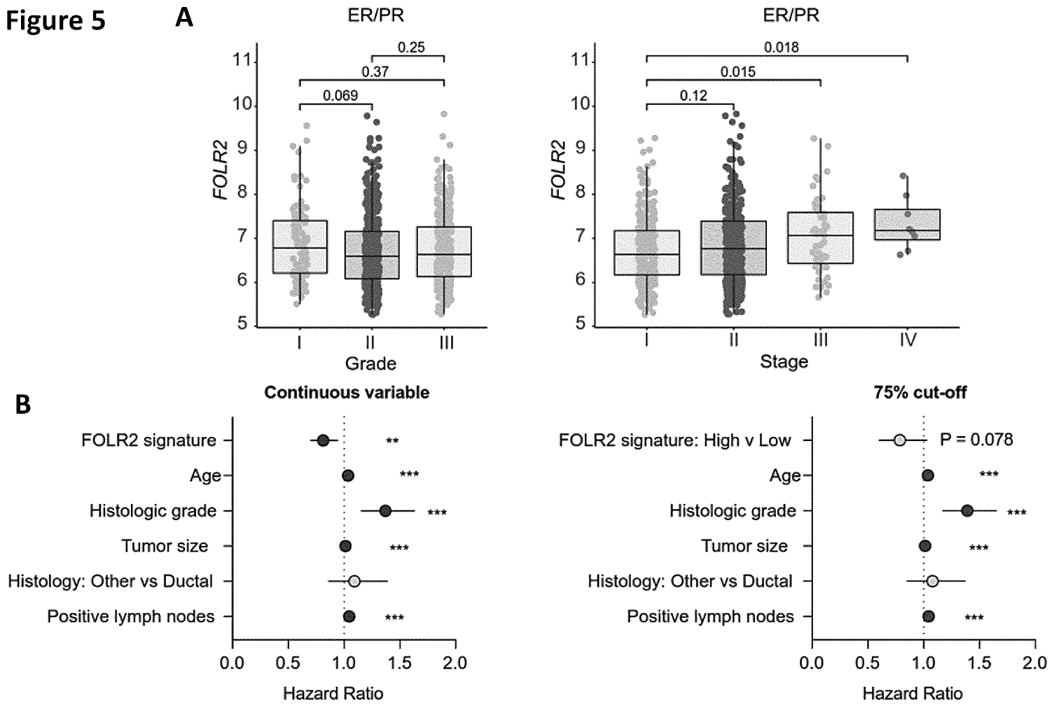

FIG. 5. FOLR2 gene-signature is an independent prognostic factor correlating with better survival
A. FOLR2mRNA expression in luminal (ER/PR) breast tumors of different grades and stages from the METABRIC dataset.
B. Multivariate analysis of the prognostic value of the FOLR2 signature as a continuous variable (left) or with 75% cut-off, adjusted age, histological grade, tumor size, histology (reference+ductal) and number od disease-positive lymph nods. Hazard ratios with 95% confidence intervals are shown. Asterisks refer to P value from the Wald test for each variable.

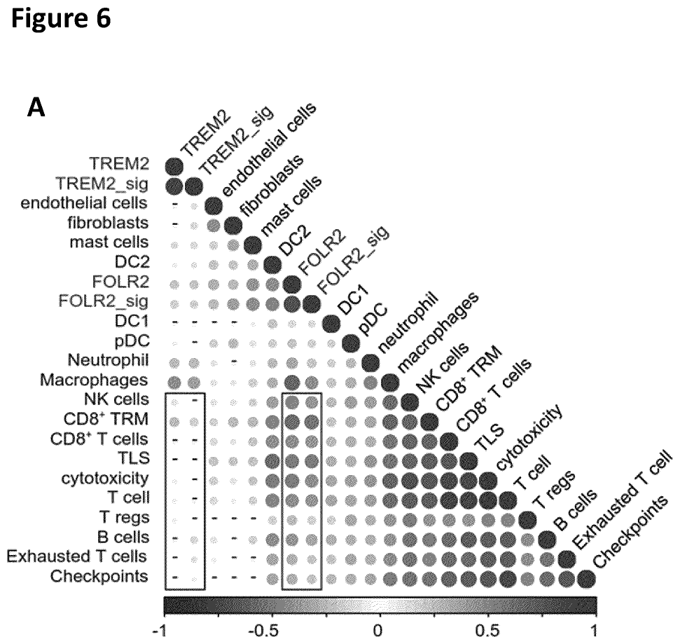

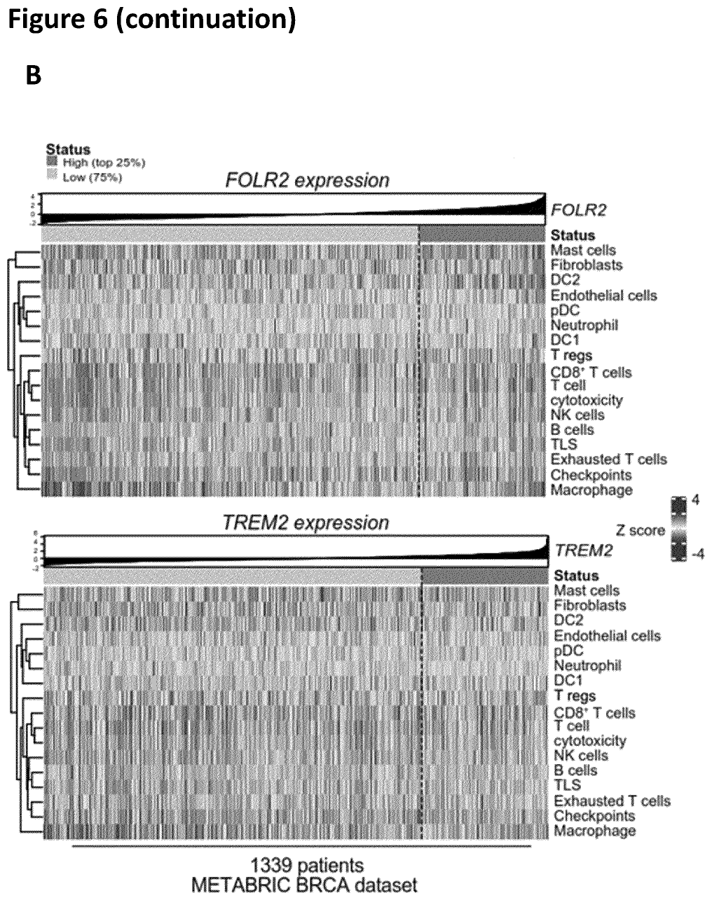

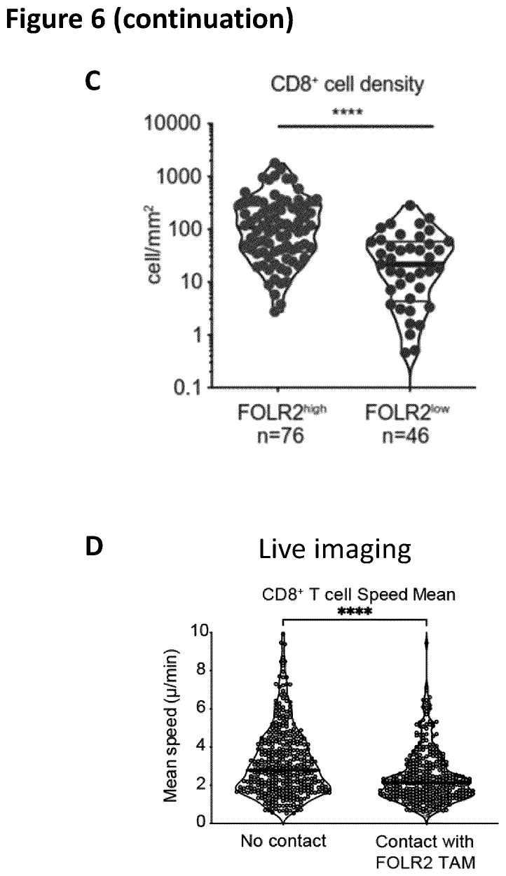

FIG. 6. FOLR2⁺ macrophages are enriched in CD8⁺ T cells infiltrated-tumors and co-localize with lymphoid aggregates across cancers.
A-B. Correlation map (A) and heatmaps (B) analyzing the association of FOLR2 and TREM2 genes to immune cell gene signatures in the METABRIC dataset.
C. Density of CD8⁺ T cells in tumors with high or low FOLR2⁺ cell density determined on immunofluorescence images of tissue-microarray.
D. Graph of the average speed of endogenous CD8⁺ T cells in contact or not with FOLR2⁺ macrophages determined by Live imaging. Mann-Whitney test.

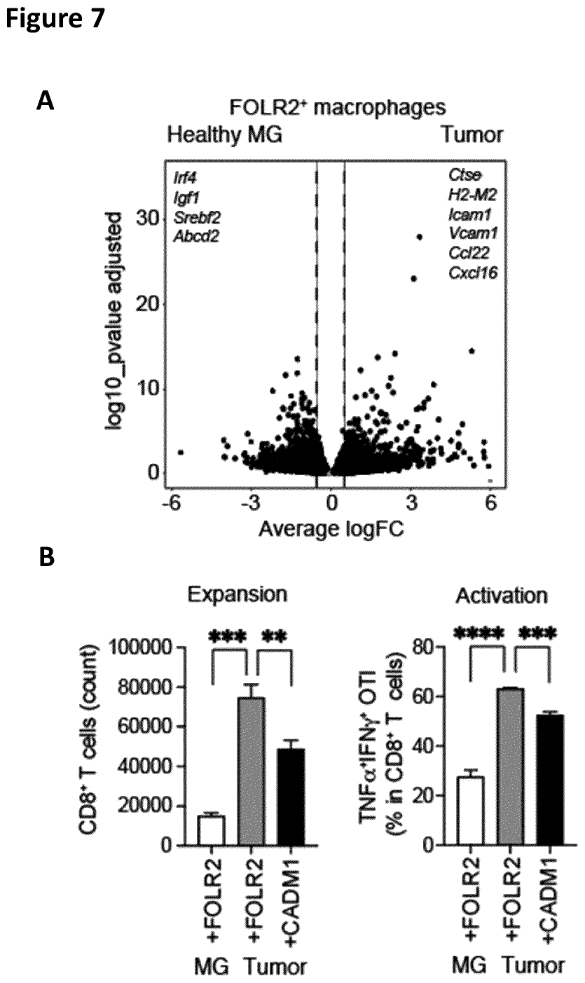

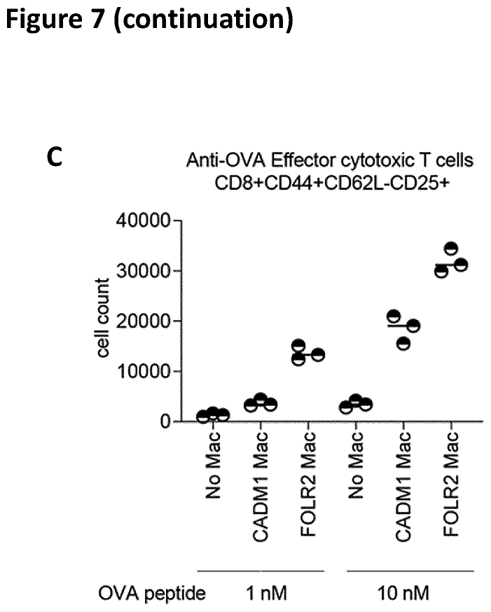

FIG. 7. FOLR2 macrophages can promote T cell effector differentiation.
A. Volcano plot showing DEG between FOLR2⁺ macrophages isolated from healthy MG or mammary tumors.
B. SIINFEKL OVA peptide pulsed FOLR2⁺ or CADM1⁺ macrophages were co-cultured with CTV labelled naïve OTI CD8⁺ T cells. T cell proliferation and activation were assessed by flow cytometry after 3 days. Data in the right panel represent OTI T cell counts and % of IFN-γ⁺TNF-α⁺CD8⁺ T cells after 3 days co-culture with the distinct macrophage populations pulsed with 1 nM of SIINFEKL. Data are mean+/−SD of triplicate wells and representative of 2 experiments. Unpaired t test.
C. FOLR2 macrophages were purified from mouse mammary tumors and loaded with OVA peptide. After washing, FOLR2 macrophages were co-cultured for 3 days with anti-OVA naive CD8 T cells. At day 3 after culture, activation and proliferation of T cell was measured. FOLR2 macrophage show higher capacity to activate T cells than CADM1 macrophages or OVA alone.

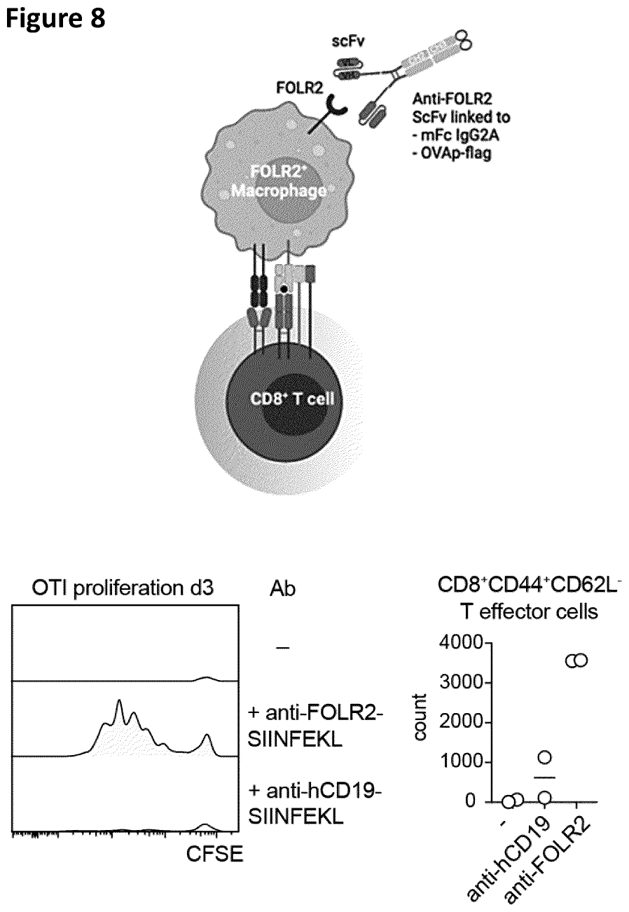

FIG. 8. Vaccination using Anti-FOLR2 targeting antibody coupled to a model antigen elicit a specific CD8+ T cell response
Scheme of anti-FOLR2 ScFv linked to SIINFEKL peptide. WT mice were immunized with anti-FOLR2-SIINFEKL or anti-hCD19-SIINFEKL as control. 4 days later, CFSE labeled CD45.1 OTI naive CD8⁺ T cells were adoptively transferred into immunized mice and their proliferation (CFSE dilution) and activation (CD44 expression and CD62L downregulation) was assessed 3 days later in lymph nodes draining the immunization site.

### EXAMPLES

### A. Material and Methods

### 1. Experimental Models and Subject Details

### Human Tumor Samples

Primary tumors, juxta-tumor tissues, metastatic and non-metastatic tumor-draining LNs were surgically resected from luminal breast cancer patients at the Institut Curie Hospital (Paris, France), in accordance with institutional ethical guidelines and informed consent was obtained (approved by the Ethical Committee of Curie Institute, CRI-0804-2015). Lymph node metastases were assigned by the pathology department of Institut Curie and further confirmed by using pan-cancer/CD45 immune detection by flow cytometry. FOLR2 and TREM2 Immunohistochemistry was performed on tumor sections retrieved from the archive of the Pathology Unit, ASST Spedali Civili di Brescia.

### Mice

Transgenic PyMT mice (MMTV-PyMT^{634Mul})(Davie et al., 2007) were maintained on C57Bl/6 background and were bred and maintained in specific pathogen-free in Curie Institute animal facility in accordance with Curie Institute guidelines. Healthy C57BL/6J female mice were obtained from Charles River Laboratories, maintained in a non-barrier facility and included at 8-12 weeks of age for experimental procedures. Animal care and use for this study were performed in accordance with the recommendations of the European Community for the care and use of laboratory animals (2010/63/UE). Experimental procedures were specifically approved by the Ministère de l'Enseignement Supérieur et de la Recherche (authorization number 2016-06.150) in compliance with the international guidelines.

### 2. Method Details

### Human Samples Processing

Patient samples were processed as previously described (Núñez et al., 2020). In brief, freshly resected human samples were cut into small fragments and digested with 0.1 mg/ml Liberase TL (Roche) and 0.1 mg/ml DNase (Roche) in CO2-independent medium (GIBCO)+0.4 g/l of human albumin (Vialebex) for 30 min at 37° C. Single cell suspension of dissociated tissues were filtered on a 40-μm cell cell strainer (BD Biosciences), washed with CO2-independent medium+0.4 g/l of human albumin and resuspended in medium for cell counting estimation.

### Flow Cytometry Analysis

After tissue processing and cell counting, cell suspensions were stained with Live/Dead Fixable Aqua Dead Cell Stain Kit (Life Technologies) in 1× phosphate-buffered saline (PBS) for 10 minutes at room temperature. After incubation cells were washed in cold PBS+0.5% bovine serum albumin (BSA)+2 mM EDTA and stained with fluorescent antibodies (Table) in the presence of Fc-receptors blocking reagent (Miltenyi) during 30 minutes at 4° C. Cell suspensions were subsequently washed and submitted to intracellular staining using fixation/permeabilization kit (eBiosciences/Thermo Fischer) accordingly to the manufacturer's instructions. Data acquisition was performed using an LSR-Fortessa (BD), compensation and analysis were done using FlowJo software (TreeStar).

### Mass Cytometry Staining and Data Analysis

For mass cytometry, pre-conjugated or purified antibodies were obtained from Invitrogen, Fluidigm (pre-conjugated antibodies), Biolegend, eBioscience, Becton Dickinson or R&D Systems as listed in Table. For some markers, fluorophore-conjugated or biotin-conjugated antibodies were used as primary antibodies, followed by secondary labeling with anti-fluorophore metal-conjugated antibodies (such as the anti-FITC clone FIT 22) or metal-conjugated streptavidin, produced as previously described (Becher et al., 2014). Briefly, patient lymph nodes cell suspension (around 30×10⁶ cells/well in a U-bottom 96 well plate; BD Falcon, Cat #3077) were washed once with 200 mL FACS buffer (4% FBS, 2 mM EDTA, 0.05% Azide in 1× PBS), then stained with 100 mL 200 mM cisplatin (SigmaAldrich, Cat #479306-1G) for 5 min on ice to exclude dead cells. Cells were then washed with FACS buffer and once with PBS before fixing with 200 mL 2% paraformaldehyde (PFA; Electron Microscopy Sciences, Cat #15710) in PBS overnight or longer. Following fixation, the cells were pelleted and resuspended in 200 μL 1× permeabilization buffer (Biolegend, Cat #421002) for 5 min at room temperature to enable intracellular labeling. Bromoacetamidobenzyl-EDTA (BABE)-linked metal barcodes were prepared by dissolving BABE (Dojindo, Cat #B437) in 100 mM HEPES buffer (GIBCO, Cat #15630) to a final concentration of 2 mM. Isotopically-purified PdCl2 (Trace Sciences) was then added to the 2 mM BABE solution to a final concentration of 0.5 mM. Similarly, DOTA-maleimide (DM)-linked metal barcodes were prepared by dissolving DM (Macrocyclics, Cat #B-272) in L buffer (MAXPAR, Cat #PN00008) to a final concentration of 1 mM. RhCl3 (Sigma) and isotopically-purified LnCl3 was then added to the DM solution at a final concentration of 0.5 mM. Six metal barcodes were used: BABE-Pd-102, BABE- Pd-104, BABE-Pd-106, BABE-Pd-108, BABE-Pd-110 and DMLn-113. All BABE and DM-metal solution mixtures were immediately snap-frozen in liquid nitrogen and stored at 80C. A unique dual combination of barcodes was chosen to stain each tissue sample. Barcode Pd-102 was used at a 1:4000 dilution, Pd-104 at a 1:2000, Pd-106 and Pd-108 at a 1:1000, and Pd-110 and Ln-113 at a 1:500. Cells were incubated with 100 mL barcode in PBS for 30 min on ice, washed in permeabilization buffer and then incubated in FACS buffer for 10 min on ice. Cells were then pelleted and resuspended in 100 mL nucleic acid Ir-Intercalator (MAXPAR, Cat #201192B) in 2% PFA/PBS (1:2000), at room temperature. After 20 min, cells were washed twice with FACS buffer and twice with water before being resuspended in water. In each set, the cells were pooled from all tissue types, counted, and diluted to 0.5×106 cells/mL. EQ Four Element Calibration Beads (DVS Science, Fluidigm) were added at a 1% concentration prior to acquisition. Cell data were acquired and analyzed using a CyTOF Mass cytometer (Fluidigm). The CyTOF data were exported in a conventional flow-cytometry file (.fcs) format and normalized using previously-described software (Finck et al., 2013). Events with zero values were randomly assigned a value between 0 and −1 using a custom R script employed in a previous version of the mass cytometry software (Newell et al., 2012). Cells for each barcode were deconvolved using the Boolean gating algorithm within FlowJo. The CD45⁺Lin(CD3/CD19/CD20)⁻HLA-DR⁺ population of PBMC were gated using FlowJo and exported as a .fcs file.

### Human and Mouse Sample Processing for Single Cell Sequencing

Mouse tumors from PyMT or wild-type littermate control were cut in small pieces and digested for 1 h at 37° C. in CO2-independent medium (Gibco) containing 150 μg/mL DNAse and 75 μg/mL Liberase TL (Roche). Cell suspensions were then obtained by filtering on a 40 μM cell-strainer (BD). Red blood cells were subsequently lysed with red blood lysis buffer. Finally, cells were resuspended in cold PBS containing 2 mM EDTA (Invitrogen) and 5% bovine serum albumin (BSA; Sigma) for further assays.

Human tumors were processed according to previously published method (Leruste et al., 2019)(Bourdely et al., 2020). Briefly, after tissue processing, dissociation and cell counting, cell suspensions were maintained on ice and stained for FACS-sorting with antibodies depicted on Table and DAPI. Cells were isolated using FACS-ARIA III (BD) cell sorter and collected in cold PBS+0.04% of BSA for cell counting. PBMC were obtained from fresh blood samples by density gradient centrifugation using Lymphoprep (Stemcell Technologies) according to the manufacturer instructions, then washed and resuspended in CO2-independent medium+0.4 g/l of human albumin prior FACS-sorting. Blood monocytes were also collected in cold PBS+0.04% of BSA before cell counting. All tissues were processed within 1 hour after tumor resection, and sorted cells were loaded in a 10× Chromium chip instrument within 6 hours.

### Single-Cell RNA-Sequencing

Human and mouse cellular suspensions (3000 to 8000 cells) were loaded on a 10× Chromium Controller (10× Genomics) according to manufacturer's protocol based on the 10× GEMCode proprietary technology. Single-cell RNA-Seq libraries were prepared using Chromium Single Cell 3′ v2 or v3 Reagent Kit (10× Genomics) according to manufacturer's protocol. Briefly, the initial step consisted in performing an emulsion where individual cells were isolated into droplets together with gel beads coated with unique primers bearing 10× cell barcodes, unique molecular identifiers (UMI), and poly(dT) sequences. Reverse transcription reactions were engaged to generate barcoded full-length cDNA followed by the disruption of emulsions using the recovery agent and cDNA clean up with DynaBeads MyOne Silane. Bulk cDNA was amplified using a GeneAmp PCR System 9700 with 96-Well Gold Sample Block Module (Applied Biosystems) (98° C. for 3 min; cycled 11/12×: 98° C. for 15 s, 63° C. for 20 s and 72° C. for 1 min; held at 4° C.). Amplified cDNA product was cleaned up with the SPRI select Reagent Kit (Beckman Coulter). Indexed sequencing libraries were constructed using the reagents from the Chromium Single Cell 3′ v3 Reagent Kit, following these steps: (1) fragmentation, end repair, and a-tailing; (2) size selection with SPRI select; (3) adaptor ligation; (4) post ligation cleanup with SPRI select; (5) sample index PCR and cleanup with SPRI select beads. Library quantification and quality assessment was performed using Qubit fluorometric assay (Invitrogen) with dsDNA HS (High Sensitivity) Assay Kit and Bioanalyzer Agilent 2100 using a High Sensitivity DNA chip (Agilent). Indexed libraries were pooled according to number of cells and sequenced on a NovaSeq 6000 (Illumina) using paired-end 28×91 bp. A depth around 50,000 reads per cell was obtained.

### Single-Cell RNA-Sequencing Data Processing and Analysis

Alignment and Raw Expression Matrix Construction. Human and mouse raw sequencing data were respectively aligned on reference genome GRCh38/84 and GRCm38/84 (genome assembly/ENSEMBL release) using 10× software CellRanger (Version 3.0.2) with default parameters. Gene expression counts for individual cells were generated using cellranger count. Cell Selection, Filtering and Normalization. For both human and mouse raw count matrix, cells expressing at least 200 genes were kept. Cells with mitochondrial content greater than 10% were removed. After this quality control, data were normalized by total counts following the Seurat 3 R pipeline. Cells identified as contaminant of the gating strategy or with high heat-shock-protein or ribosomal coding genes content were filtered out. Variable Gene Selection and Sample Merging. For sample merging a VST (Variance Stabilizing Transformation) method selecting for the most variable genes was applied. The Seurat V3 integration pipeline was performed using the most 8000 genes for the ten human samples. The 3000 most variable genes were used to merge the two mice samples. For both anchors selection and integration steps, the default parameters of Seurat V3 functions were used. Dimensionality reduction and Visualization. Data were scaled by applying a regression using as variation factors, the total UMI counts, the percent of expressed mitochondrial genes, the origin sample and tissue of each cell and the version of CellRanger chemistry kit used for sequencing. Heatmaps are showing z-scores of this scaled matrix. The UMAP visualization was built using respectively the 50 and 30 most informative components of the PCA for Human and Mouse. Clustering and Differential Gene Expression Analysis. The clustering was processed by constructing a Shared Nearest Neighbor (SNN) Graph. The 20 neighbors of each cell were first determined. The resulting KNN graph was used to construct the SNN graph by calculating the neighborhood overlap (Jaccard index) between every cell and its 20 nearest neighbors. Clustering was then applied on this graph using the Louvain graph-based algorithm. Differential gene expression analysis was applied on each sample log normalized matrix. The Seurat function FindAllMarkers was used with a Logistic Regression test, and adding as variation factors, the origin sample and tissue of each cell and the version of CellRanger sequencing kit used. Only genes expressed in more than 10% of the cells in a cluster and having at least 0.10 of log Fold-Change between compared groups were tested. Low signals produced by genes with dropouts were able to be detect. For the volcano plots only the first condition was kept. At the end, only genes with a significative adjusted p-value (pv<0.05, false discovery rate (FDR) adjusted p-value) were kept and used to define each cluster.

Human-Mouse Merging and Seurat Label Transfer Prediction Score. To compare mouse and human macrophages, conserved orthologue genes between both species were selected. Corresponding gene symbols table was provided by the Mouse Genome Informatics database. For genes with more than one corresponding orthologue in the other species, the mean expression of all orthologues was taken. Seurat label transfer scoring algorithm was applied from mouse (as query) to human dataset (as reference). For the anchors searching step, the 10 000 most variable genes of the reference dataset were used. The following steps of the Seurat pipeline was applied with default parameters.

Processing of published dataset. The dataset of Azizi et al, Cell 2018 (GSE114725) was downloaded. The classical pipeline for single cell analysis of Seurat V3 (without integration correction) from the raw count matrix (supplementary file GSE114725_rna_raw.csv.gz) was used. Next, Louvain graph- based clustering was performed. At the resolution 0.9 39 clusters were obtained: 14 clusters of T cells, 6 clusters of B cells, 5 clusters of NK cells, 1 cluster of pDCs, DC1, DC2, CD16⁺ monocytes, CD14⁺ monocytes, neutrophils or mast cells, 3 clusters of macrophages and 4 clusters of contaminating cells. Clusters of the same immune cell types were merged, except for the macrophages clusters and contaminating clusters were removed. The dataset of Han et al, Cell 2018 (GSE108097) were downloaded. 2 samples of virgin mammary gland and 1 sample of pregnant mammary gland were integrated from the raw data (supplementary file GSE108097_RAW.tar). The same pipeline described above including the integration step was used.

### Bulk RNA-Sequencing

After tissue processing and cell count, cell suspensions were washed in cold PBS+0.5% BSA+2 mM EDTA and submitted to surface antibodies staining (Table) in the presence of Fc-receptors blocking (FcR Blocking Reagent; Miltenyi) for 30 minutes at 4° C. Myeloid cell subsets were isolated using FACS-ARIA III (BD) cell sorter and directly collected on lysing TCL buffer (QIAGEN) containing 1% of beta-mercaptoethanol before storage at −80° C. RNA were extracted and isolated using the Single Cell RNA purification kit (Norgen, Cat #51800) according to the manufacturer's instructions. After extraction, total RNA was analyzed using Agilent RNA 6000 Pico Kit on the Agilent 2100 Bioanalyzer System. RNA quality was estimated based on capillary electrophoresis profiles using the RNA Integrity Number (RIN). RNA sequencing libraries were prepared using the SMARTer Stranded Total RNA-Seq Kit v2-Pico Input Mammalian (Clontech/Takara). The input quantity of total RNA was comprised between 1 and 22 ng. This protocol includes a first step of RNA fragmentation, using a proprietary fragmentation mix at 94° C. The time of incubation was set up for each sample, based on the RNA quality, and according to the manufacturer's recommendations. After fragmentation, indexed cDNA synthesis was performed. Then the ribodepletion step was performed, using probes specific to mammalian rRNA. PCR amplification was finally achieved to amplify the indexed cDNA libraries, with a number of cycles set up according to the input quantity of tRNA. Library quantification and quality assessment was performed using Qubit fluorometric assay (Invitrogen) with dsDNA HS (High Sensitivity) Assay Kit and LabChip GX Touch using a High Sensitivity DNA chip (Perkin Elmer). Libraries were then equimolarly pooled and quantified by qPCR using the KAPA library quantification kit (Roche). Sequencing was carried out on the NovaSeq 6000 (Illumina), targeting between 10 and 15M reads per sample and using paired-end 2×100 bp.

### Bulk RNA-Sequencing Data Processing and Analysis

The raw sequencing data was initially aligned on the human reference genome hg19, using STAR aligner (v2.5.3a) (Dobin et al., 2013). Raw read counts matrix made also with STAR (using the parameter—quantMode GeneCounts). FastQ files quality control were applied with FastQC (removing of adapters and low-quality bases). Non-expressed genes (the sum of counts in all samples less than 2) and lowly expressed genes (background; log2 of the average of raw counts in all samples less than 2) were removed from the raw read count matrix. For differential expression analysis, the R package DESeq2 (version 1.24.0)(Love et al., 2014) was used with a p-value correction. The DESeq matrix was designed using the information of the patient and the cell type (following the formula design=˜batch+condition). The median of ratios method (Anders and Huber, 2010) was used for the normalization, and the rlog transformation for visualization and clustering as proposed in the DESeq2 tutorial (Love et al., 2014).

### Immunohistochemistry

Paraffin-embedded tissue blocks were cut with a microtome into fine slivers of 3 microns. Immunohistochemistry was processed in a Bond RX automated (Leica) with Bond Polymer refine detection kit (Leica, DS9800). Antigen retrieval was performed in BOND Epitope Retrieval Solution 1 (Leica, AR9961). Primary antibody APOE (Abcam; ab52607) was incubated 30 minutes at room temperature. Slides were counterstained with hematoxylin before mounting with resin. Images were acquired by using Digital Pathology slide scanner (Ultra Fast Scanner 1.8, Philips). FOLR2 expression was tested on human tissues by using immunohistochemistry. Sample included reactive lymph nodes (n=7), primary carcinomas (n=47), metastatic tumor draining lymph nodes (n=7) and distant metastasis to lung (n=8) and liver (n=11) from different primary site (breast, bladder, gastro-intestinal, lung, kidney and skin) retrieved from the archive of the Pathology Unit, ASST Spedali Civili di Brescia. Briefly, anti-FOLR2 (clone OTI4G6, 1:100, ThermoFisher SCIENTIFIC) and anti-TREM2 (clone D814C, 1:100, Cell Signaling Technology) antibodies were revealed using Novolink Polymer (Leica) followed by DAB. For double staining, FOLR2 was combined with anti-CD3 (clone LN10, 1:70, Leica Biosystem), anti-CD20 (clone L26, 1:200, Leica Biosystem), anti-CD31 (clone PECAM-1, 1:50, Leica) and anti-TREM2. Briefly, after completing the first immune reaction, the second immune reaction was visualized using Mach 4 MR-AP (Biocare Medical), followed by Ferangi Blue. Localization of FOLR2⁺ cells within tertiary lymphoid structures (TLS) was confirmed by double for the B-cell marker CD20 and the T-cell marker CD3.

### Immunofluorescence on Fixed Tumor Slices

Biopsies were fixed overnight at 4° C. in a Periodate-Lysine-Paraformaldehyde solution (0.05 M phosphate buffer containing 0.1 M L-lysine [pH 7.4], 2 mg/ml NaIO4, and 10 mg/ml paraformaldehyde). Fixed tumors were then embedded in 5% low-gelling-temperature agarose (type VII-A, Sigma-Aldrich) and cut into 400 μm-thick slices as previously described (Peranzoni et al., 2018). Tumor slices were stained for 15 minutes at 37° C. with antibodies shown at Table. All antibodies were diluted in PBS and used at a concentration of 5 μg/ml. except anti-FOLR2 and CD31 antibodies that were used at 10 μg/ml. Z-stack images of 5×5 fields were taken with a 10× water immersion objective (10×/0.3 N.A.) on an inverted spinning disk confocal microscope (IXplore, Olympus). Virtual slices were reconstituted and analyzed with the ImageJ software.

### Multispectral Immunofluorescence on Paraffin-Embedded Tissues

Paraffin-embedded tissue blocks were cut with a microtome into fine slivers of 5 microns. Immunostaining was processed in a Bond RX automated (Leica) with Opal™ 7-Color IHC Kits (Akoya Biosciences, NEL821001KT) according to the manufacturer's instructions using antibodies shown at Table. Tissue sections were coverslipped with Prolong™ Diamond Antifade Mountant (ThermoFisher) and stored at 4° C. Subsequently, slides were scanned using the Vectra® 3 automated quantitative pathology imaging system (Vectra 3.0.5; Akoya Biosciences). Multispectral images were unmixed and analysed using the inForm Advanced Image Analysis Software (inForm 2.4.6; Akoya Biosciences)

### Tissue Microarray (TMA) Multispectral Immunofluorescence

Paraffin-embedded tissue microarray from breast cancer patients (n=122 spots) were obtained commercially (AMSBIO, England). Immunostaining was processed in a Bond RX automated (Leica) with Opal™ 7-Color IHC Kits (Akoya Biosciences, NEL821001KT) according to the manufacturer's instructions (Table). After immunostaining, slides were submitted to DAPI staining, washed and coverslipped with Prolong™ Diamond Antifade Mountant (ThermoFisher). Subsequently, slides were scanned using the Vectra® 3 automated quantitative pathology imaging system (Vectra 3.0.5; Akoya Biosciences). Multispectral images were unmixed using the inForm Advanced Image Analysis Software (inForm 2.4.6; Akoya Biosciences) and analyzed by HALO software for immune subsets quantification.

### METABRIC Data Analysis

METABRIC (METABRIC Group et al., 2012) gene expression data, as well as clinical and sample level metadata were downloaded from cBioPortal. Patient breast cancer subtype was annotated by defining TNBCs as those with a negative ER and HER2 status. HER2 positive patients were defined as any patients that had a HER2 positive status variable. ER/PR positive patients were defined as being HER2 negative but either ER or PR status positive. TNBCs with a positive PR IHC status were removed. Patients that died of other causes not related to their disease, as well as patients with breast sarcomas were removed. TNBC expression data was submitted to the TNBC type (Chen et al., 2012) algorithm that removed a further 6 patients from the TNBC cohort (MB-3297, MB-7269, MB-5008, MB-6052, MB-0179, MB-2993. The final cohort consisted of 1339 samples (168 TNBC, 204 HER2 and 967 ER/PR). To score individual samples for gene signatures of interest, the gene expression data was Z-score normalised, and then a mean level of expression across signature genes was calculated. MCPcounter (1.2.0) was used to infer the abundance of immune and stromal cell populations in each sample. To stratify patients according to gene signature scores, a 75% cut-off was used therefore defining 25% of patients as “high” scorers. Survival analysis was carried out using the survival (3.1-12) and survminer (0.4.6) packages in R. Pairwise Spearman correlations were computed between signatures of interest and these were plotted as a correlation heatmap using the corrplot R package (0.84). Correlations that were statistically non-significant (p<0.05) were marked with a dash (−). MCP counter-estimated immune cell abundance. Comparing MCP counter cell types between patients stratified into high and low groups by a particular signature, Wilcoxon tests were used with P values adjusted using the Benjamini-Hochberg method. ggplot2 (3.3.0), pheatmap (1.0.12) and corrplot (0.84) packages were used for plotting data.

### CPTAC Data Analysis

Data used in this publication were generated by the Clinical Proteomic Tumor Analysis Consortium (NCI/NIH). Log-ratio normalised proteomic data including clinical information and RNA sequencing data from the CPTAC BRCA study were downloaded (http://linkedomics.org/data_download/TCGA-BRCA/). BRCA patient samples annotated as either ER or PR positive, and HER2 negative, were selected for downstream analysis. Patients were then stratified according to a 25% cut-off by FOLR2 protein expression and univariate Cox regression analysis was carried out. Logrank test scores were plotted on Kaplan-Meier plots.

### GTEx to TCGA Comparison

To compare TREM2 and FOLR2 mRNA expression between non-disease healthy tissue, tumour- adjacent normal tissue, and tumour tissue, datasets from GTEx and TCGA consortia were harnessed. For breast, colon and lung studies, normalised transcriptomic data from Github (https://github.com/mskcc/RNAseqDB/tree/master/data/normalized) were downloaded. non-parametric, Wilcoxon T tests were used to compare TREM2 and FOLR2 expression between tissue types. The Genotype-Tissue Expression (GTEx) Project was supported by the Common Fund of the Office of the Director of the National Institutes of Health, and by NCI, NHGRI, NHLBI, NIDA, NIMH, and NINDS.

### Density of FOLR2+ Macrophages

Paraffin-embedded tissue microarray from breast cancer patients (n=122 spots) were obtained commercially (AMSBIO, England). Immunostaining was processed in a Bond RX automated (Leica) with Opal™ 7-Color IHC Kits (Akoya Biosciences, NEL821001KT) according to the manufacturer's instructions. After immunostaining, slides were submitted to DAPI staining, washed and coverslipped with Prolong™ Diamond Antifade Mountant (ThermoFisher). Subsequently, slides were scanned using the Vectra® 3 automated quantitative pathology imaging system (Vectra 3.0.5; Akoya Biosciences). Multispectral images were unmixed using the inForm Advanced Image Analysis Software (inForm 2.4.6; Akoya Biosciences) and analyzed by HALO software for immune subsets quantification.

### Recombinant Anti-FOLR2 IgG Construct Linked to Polyepitope

Recombinant murine IgG2a anti murine FOLR2 construct linked to polyepitope comprising the well-characterized CD8+ T cell epitope SIINFEKL (SEQ ID NO: 1) from ovalbumin (OVA) was derived from anti-FolR2 scFv disclosed in Nagai et al. (Arthritis and Rheumatism, 2006, 54, 3126-3134). The scFv linked to the polyepitope was fused to an IgG2a Fc mutated (N297A) not to bind high affinity Fc-gamma receptor. The polynucleotide construct (SEQ ID NO: 2; 1812 nt) comprises the following elements:
Kozak consensus: positions 1 to 6
Signal peptide: positions 7 to 63
XhoI site: positions 64 to 69
anti-mouse FOLR2 VL: positions 70 to 408
(G4S)4linker: positions 409 to 468
Anti-mouse FOLR2 VH: positions 469 to 831
G4S(A)3linker: positions 832 to 855
NotI-Bgl II sites: positions 856 to 870
FcIgG2A*N297A: positions 871 to 1569
srfI site: positions 1570-1578
polyepitope: positions 1579 to 1770
AscI site: positions 1771 to 1779
FlagTAG: positions 1780 to 1803
Stop codon: positions 1804 to 1806
NheI site: positions 1807 to 1812.

Recombinant anti-FOLR2 IgG protein construct corresponds to SEQ ID NO: 3.

### Statistical Analysis

The tests used for statistical analyses are described in the legends of each concerned figure and have been performed using GraphPad Prism v8 or R v3.4. Symbols for significance: ns, non-significant; *, <0.05, **, <0.01; ***, <0.001; ****, <0.0001. For each experimental group, n represent the number of subjects within each group.

### B. Results

The inventors have implemented scRNAseq of tumor associated CD14⁺HLA-DR⁺ cells isolated from metastatic LNs and primary breast tumors to assess the cellular heterogeneity within the CD14⁺ compartment. They identify two phenotypically distinct macrophage populations: TREM2⁺ macrophages expressing Triggering Receptor Expressed by Myeloid cells-2 (TREM2), Osteopontin (SPP1) and Cell Adhesion Molecule 1 (CADM1) genes; FOLR2⁺ macrophages expressing Folate Receptor 2 (FOLR2), selenoprotein P (SEPP1), solute carrier family 40 member 1 (SLC40A1), Hyaluronan receptor (LYVE-1) and Mannose Receptor C-Type 1 (MRC1/CD206) genes. This study shows that tumor-associated TREM2⁺ and FOLR2+ macrophages are evolutionarily conserved between human and mouse breast cancers. TREM2³⁰ macrophages are poorly represented in healthy breast tissues but increase with tumor development. By contrast, the study shows that FOLR2⁺ macrophages are tissue-resident macrophages (TRMs) populating healthy mammary glands prior the onset of cancer development. Specific gene signatures defining FOLR2⁺ macrophages correlate with better relapse-free survival in breast cancer patients. Accordingly, FOLR2⁺ macrophages positively correlate with signatures of major cellular players of anti-tumor immunity, including CD8⁺ T cells, NK cells and dendritic cells (DCs). This study further shows that FOLR2⁺ macrophages locate near vessels and cluster with CD8⁺T cell aggregates in tumor resection samples. This FOLR2⁺ macrophage/CD8⁺ T cell co-localization correlates with favorable clinical outcomes suggesting an anti-tumorigenic role for this newly characterized macrophage subset.

### 1. APOE Expression Defines Tumor-Associated Macrophages in Human Breast Cancer

Motivated by the idea of unambiguously identifying macrophages within breast tumors, it was sought to define common features enabling the distinction between breast cancer macrophage populations and infiltrating CD14⁺CD1c⁻ monocytes, CD14⁺CD1c⁺ inflammatory DCs/DC3 or CD14⁻CD1c⁺cDC2 (Villani et al., 2017)(Bourdely et al., 2020)(Dutertre et al., 2019).

In a first approach, mononuclear phagocytes of matched primary tumors, non-metastatic and metastatic lymph nodes (LNs) were quantified from a cohort of 13 treatment-naive luminal breast cancer patients. It was found that CD1c⁻CD14⁺ monocyte/macrophage population increased most significantly in metastatic LNs as compared to matched non-metastatic LNs (FIG. 1A). This positive correlation between CD14⁺ cell infiltration and tumor invasion, was confirmed in a second cohort of patients. CD14⁺ cell infiltration correlated with the extent of tumor invasion in LNs (FIG. 1B).

It was next sought to characterize the heterogeneity within the entire tumor-infiltrating CD14⁺ cells in an unbiased manner. To this end, metastatic LNs, primary tumors and blood of untreated luminal breast cancer patients were analyzed (FIG. 1C). All mononuclear phagocytes were isolated by FACs-sorting the CD11c⁺HLA-DR⁺ cell fraction and performing scRNAseq using a droplet-based approach. The SEURAT pipeline was used to process the data (FIG. 1D). Contaminating lymphocytes (B/T/NK cells), XCR1⁺ DCs and CCR7⁺LAMP3⁺DCs (Zhang et al., 2019) were excluded from the analysis. The ˜18000 remaining myeloid cells from all the patients were then merged (FIG. 1D). Louvain Graph-based clustering identified 4 clusters of mononuclear phagocytes and populations of cycling (mKI67, TOP2A, CDC20, e.g.) and “stressed” cells (HSPA1A, HSPB1, e.g.). Cluster 0 was characterized by the selective and high expression of markers (S100A8, S100A9, S100A12, VCAN) defining CD14⁺CD16⁻ monocytes (Villani et al., 2017)(FIG. 1D, E). Cluster 1 was characterized by genes defining CD1c⁺DCs (including DC2 and DC3 subsets) while cluster 4 was identified as CD14⁻CD16⁺ monocytes (Villani et al., 2017)(Dutertre et al., 2019)(Bourdely et al., 2020). Monocyte-clusters (c0 and c4) were both found in blood, tumor and metastatic LNs. The remaining cluster 2 was identified as TAMs because it selectively expressed high levels of a TAM signature (FIG. 1F)(Azizi et al., 2018). Cluster 2 expressed high levels of APOE, APOC1, C1QA, C1QC enabling the distinction from monocytes (FIG. 1E). It is shown that homogenous expression of APOE selectively discriminate TAMs from both CD14⁺ monocytes and CD1c⁺ DCs (FIG. 1F). No other commonly used markers (CSF1R, CD68, CD14) achieved this discrimination. CD68 is expressed in CD14⁺ monocytes, CD16⁺ monocytes and CD1c DCs; CD14 is expressed by monocytes and a subset of CD1c⁺ DCs; CSF1R is promiscuous (FIG. 1F).

It was next wanted to validate protein expression distinguishing macrophages/TAMs from monocytes within CD1c⁻CD14⁺ cells. The intracellular proteins S100A8 and S100A9—found to be highly and specifically expressed in CD14⁺ monocyte cluster 0—followed the protein-expression pattern of cell-surface CCR2, which was not detected in this scRNAseq dataset. The best monocyte/macrophage discrimination was obtained by staining with APOE and CCR2 (FIG. 1G). Then, the contribution of CCR2⁺ monocytes versus APOE⁺ macrophages to the CD14⁺ cell compartment within luminal breast tumor lesions (primary tumor or highly invaded metastatic LNs) versus tumor-free tissues (blood, healthy breast tissue, tumor-free and lowly invaded metastatic LNs or juxta-tumor) was analyzed. It was found that the frequency of APOE⁺ macrophages increased with tumor burden while the frequency of CCR2⁺ monocytes decreased (FIG. 1G). Finally, it was found that APOE⁺ cells located near and inside the tumor lesions in metastatic LNs and primary tumors. Altogether, these results establish APOE as a specific marker to identify macrophages in primary and metastatic luminal breast cancer.

### 2. Single Cell RNA Sequencing Reveals Two Main Subsets of APOE⁺ Macrophages

Next, the heterogeneity of APOE⁺ TAMs (cluster 2) was investigated. Louvain graph-based clustering identified 3 clusters of TAMs (FIG. 2A). Hierarchical clustering showed that clusters 0 and 1 were closer to each other as compared to cluster 2 (FIG. 2B). To further explore the transcriptional heterogeneity found within the APOE⁺ macrophages, single-cell regulatory network inference and clustering (SCENIC) was implemented to study the gene regulatory network (GRN) of each macrophage cluster. GRN analysis and their activity-based hierarchical clustering revealed that cluster 0 and 1 shared around half of their transcriptional regulon including CEBPB and BHLHE41 while cluster 2 presented mostly unique transcriptional regulons like NR1H3 and MAF (FIG. 2C). Analysis of differentially expressed genes revealed that FOLR2, SEPP1, SLC40A1, MRC1, LYVE1, discriminated cluster 2 from both cluster 0 and cluster 1 (FIG. 2D). Conversely, TREM2, SPP1, ISG15 discriminated both cluster 0 and cluster 1 from cluster 2 (FIG. 2D). Altogether, it is shown that FOLR2 is a defining marker for cluster 2 while TREM2 defines cluster 0 and 1 (FIG. 2E). It is concluded that APOE⁺ TAMs comprise two distinct populations: the TREM2⁺ macrophages and the FOLR2⁺ macrophages.

It was next sought to validate this finding by prospective isolation of these populations for bulk transcriptome analysis. To this end, surface proteins differentially expressed between the two populations were searched for, thus enabling their isolation by flow cytometry. TREM2 was failed to be detect at the cell surface of TAMs after tissue dissociation. Alternatively, it was found that cluster 1 (TREM2^{high} TAMs) specifically expressed and stained positive for CADM1 (FIG. 2F, G).

Flow cytometry analysis of FOLR2 and CADM1 expressions revealed mutually exclusive expression patterns (FIG. 2G). FOLR2⁺CADM1⁻ and FOLR2^{low}CADM1⁺ TAMs were isolated from both primary tumors and metastatic LNs by FACs sorting. FOLR2⁺CADM1⁻ TAMs presented a typical macrophage shape and were filled with vacuoles. In contrast, FOLR2^{low}CADM1⁺ TAMs were smaller in size, with a morphology closer to monocytes (FIG. 2G).

Next bulk RNAseq was performed on FOLR2⁺CADM1⁻ TAMs, FOLR2^{low}CADM1⁺ TAMs and CD14⁺CCR2⁺ monocytes (FIG. 2H). Hierarchical clustering showed that FOLR2⁺CADM1⁻ macrophages from both primary tumors and invaded LNs cluster together away from FOLR2^{low}CADM1⁺ macrophages or CD14⁺CCR2⁺ monocytes (FIG. 2H). The scRNAseq results were confirmed showing that FOLR2⁺ macrophages expressed higher levels of FOLR2, SEPP1, SLC40A1 and LYVE1 (FIG. 2I) as compared to FOLR2^{low}CADM1⁺ TAMs and CD14⁺CCR2⁺ monocytes. Overall, FOLR2^{low}CADM1⁺ macrophages from primary tumors clustered together with CD14⁺CCR2⁺ monocytes (FIG. 2H). However, FOLR2^{low}CADM1⁺ macrophages specifically expressed TREM2 and genes found to be overexpressed in cluster 1 (C3, FN1, SPP1) of the scRNAseq analysis (FIG. 2J).

Some of these phenotypic differences were confirmed by CyTOF profiling of CD14⁺CCR2⁻ macrophages from invaded LNs. Co-expression of LYVE1, MRC1/CD206 and CD163 was found in a macrophage population distinct from CADM1⁺ expressing macrophages (FIG. 2K).

Altogether, these results show that breast TAMs comprise two populations separable by their mutually exclusive expression of TREM2/CADM1 and FOLR2.

### 3. FOLR2⁺ Macrophages are Tissue-Resident Macrophages

A recent study identifies the infiltration of TREM2⁺ macrophages as an event associated to cancer development (Molgora et al., 2020). These data establish their transcriptional proximity to CD14⁺CCR2⁺ monocytes in breast tumors (FIG. 2H). These results suggest that TREM2⁺CADM1⁺ TAMs arise from infiltration of circulating monocytes during tumor progression.

The origin of FOLR2⁺ macrophage is not known. It was wondered whether FOLR2⁺ macrophages correspond to mammary TRMs (i.e. present in healthy breast) or tumor-recruited monocyte-derived macrophages like the TREM2⁺ TAMs. To address this question, FOLR2⁺ macrophages were quantified by flow cytometry in healthy tissues (healthy breast, mammary tissues adjacent to tumor lesion- juxta- tumor-, tumor-free or lowly-invaded metastatic LNs) versus luminal breast tumor lesion (primary tumors and highly invaded metastatic LNs). It was found that among APOE⁺ macrophages, FOLR2⁺ macrophages were enriched in healthy and juxta-tumor tissues (FIG. 3A). By contrast, the fraction of FOLR2⁻ TAMs (comprising TREM2⁺ TAMs) dramatically increased in both primary and metastatic tumor lesions as compared to healthy tissues (FIG. 3A).

The enrichment of FOLR2⁺ macrophages in adjacent normal tissue versus tumor lesions was also confirmed at the transcriptional level by analyzing breast cancer samples from the Cancer Genome Atlas (TCGA) database (FIG. 3B). FOLR2 transcripts were enriched in normal adjacent tissues as compared to breast cancer tumor lesions of different subtypes (Her2⁺, TNBC, Luminal). FOLR2 transcripts were also enriched in non-disease healthy tissues as compared to tumor in breast cancer In contrast TREM2 transcripts were enriched in breast tumor lesions as compared to tumor-adjacent normal and non-disease healthy tissues (FIG. 3B). Importantly, immunohistochemistry (IHC) analysis revealed that FOLR2⁺ macrophages were present in peri-tumoral areas in all subtypes of breast cancers. Bulk RNAseq (FIG. 2J) and CyTOF (FIG. 2K) analysis of FOLR2⁺ macrophages show that FOLR2⁺ macrophages specifically express the hyaluronan receptor LYVE1 and the mannose receptor (MRC1/CD206), both markers of perivascular macrophages (Lin et al., 2006)(Lim et al., 2018)(Chakarov et al., 2019). Therefore, it was wondered whether FOLR2⁺ macrophages would locate near vessels. Confocal imaging on tumor surgical resection specimens showed that FOLR2⁺CD206⁺ macrophages located near CD31⁺ vessels in both tumor and adjacent tissue. Altogether these results show that FOLR2⁺ macrophages are perivascular TRMs associated with healthy mammary glands and LNs.

Next mammary gland macrophages were analyzed in the mouse model enabling to carry out a longitudinal analysis of immune populations in steady state and during tumor progression. First, a published scRNAseq dataset performed on hematopoietic cells from healthy mammary glands (Han et al., 2018) was analyzed. a subset of TRMs co-expressing Folr2, Mrc1 and Lyve1, like human FOLR2⁺ macrophages was identified. These cells align to previously described MRC1⁺LYVE1⁺ TRMs (Franklin et al., 2014)(Jäppinen et al., 2019)(Wang et al., 2020). Next, scRNAseq was performed on CD45⁺CD3⁻CD19⁻B220⁻NKP46⁻ cells isolated from MMTV-PyMT autochthonous luminal-like mammary tumor model (Franklin et al., 2014)(Davie et al., 2007)(FIG. 3C). These cells were excluded from the analysis: contaminating lymphocytes (not shown), Ly6c2⁺ monocytes (c3), Ly6c2⁻Nr4a1^{high} monocytes (c6), cycling cells (c4) and cells with high content of ribosomal genes (c1). Among the remaining Fcgr1⁺ cells, 3 clusters of TAMs were identified in 23 weeks-old PyMT mouse-tumors (FIG. 3C). Among these, two expressed Cadm1 (Cx3cr1^{int} cluster 0 and Cx3cr1^{high} cluster 1, FIG. 3C). In addition, a discrete population of Folr2⁺Mrc1⁺ macrophages was identified (cluster 2, FIG. 3C). Mouse and human FOLR2⁺ TAMs share the expression of FOLR2, MRC1, SLC40A1 and MAF (FIG. 3D). On the other hand, Cadm1⁺Cx3cr1⁺ mouse macrophages (clusters 0 and 1) resemble human CADM1⁺TREM2^{high} TAMs (cluster 1, FIG. 2A) and share the expression of CADM1, HAVCR2, IF144 (FIG. 3D). Of note, the Trem2 expression pattern is more conspicuous in murine as compared to human macrophages. To probe the similarity between human and mouse FOLR2⁺ macrophages, a similarity analysis was performed across whole transcriptomes at the level of each cell (FIG. 3E). This unbiased analysis confirmed the marker-based alignment of murine Folr2⁺ macrophage to human FOLR2⁺ macrophages. Conversely, the Cadm1⁺Cx3cr1⁺ murine macrophages (clusters 0 and 1) presented high similarity with CADM1⁺TREM2⁺ human macrophages. Thus, it was concluded that FOLR2⁺ macrophages are evolutionarily conserved between murine and human luminal mammary tumors.

Next the dynamics of FOLR2⁺ (and CADM1⁺) macrophages was analyzed longitudinally during the development of murine breast cancer. To this end, mammary gland macrophages were analyzed in healthy littermate (WT), pre-lesion PyMT mice (8 weeks-old), neoplastic lesions (12 weeks-old mice), early carcinoma (14 weeks-old mice) and advanced carcinoma (20 weeks-old mice). It was found that FOLR2⁺ macrophages constitute around 90% of total macrophages (CD45⁺Lin⁻Ly6C⁻F4/80⁺CD64⁺), in healthy mammary gland (WT). The frequency of FOLR2⁺ macrophages progressively decreased upon carcinoma progression to reach a minimum of 10-20% in advanced-carcinoma lesion of 20 weeks-old PyMT mice (FIG. 3F). In contrast, carcinoma development was accompanied by the de novo expansion of CADM1⁺ macrophages representing up to 80% of total macrophage in 20 weeks-old PyMT mice (FIG. 3F). Altogether it is concluded that FOLR2⁺ macrophages represent an evolutionarily conserved TRM subset persisting in advanced carcinoma.

4. FOLR2⁺ Macrophages Correlate with Increased Survival in Breast Cancer Patients and Patients with at Least 6 Other Cancer Types

TAMs are generally thought to promote tumor growth and inhibit anti-tumor immunity. This is particularly well established in mouse models where macrophages display a plethora of pro-tumoral function (Lin et al., 2006)(Qian et al., 2011)(Franklin et al., 2014)(Linde et al., 2018). In human, TAMs generally correlate with poor prognosis and higher tumor grade. However, clinical studies have probed the association of TAMs to patient survival by using markers often shared by monocytes and macrophages (CSF1R, MRC1/CD206, CD163 e.g) or even shared with other immune cells types like pDCs (CD68 e.g.) (Colonna et al., 2004). Therefore, it was wondered whether FOLR2⁺ macrophages are similarly associated with worse survival in breast cancer patients.

To this end, gene signatures were defined enabling to infer the abundance of total macrophages or FOLR2⁺ macrophage subset within bulk tumor RNA sequencing. Three genes (C1QA, C1QB, C1QC) define a core macrophage signature shared by the 3 macrophage clusters identified in this study (FIG. 2A, FIG. 4A) and suffice to distinguish macrophages from other leukocytes lineages (FIG. 4A, B). Three genes (FOLR2, SEPP1, SLC40A1) uniquely distinguish FOLR2⁺ macrophages from other TAMs and other leukocytes lineages (FIG. 4A, B)(Azizi et al., 2018). LYVE1 was not considered for the signature analysis because of its endothelial expression.

The representation of these signatures was analyzed within bulk transcriptomes of luminal breast cancer (1339 patients, METABRIC cohort)(METABRIC Group et al., 2012). Survival probability was calculated within highest signature expressers (top 25%) versus the rest of the patients (FIG. 4C). In accordance with previous reports (Ruffell and Coussens, 2015), it was found that the highest level of macrophage infiltration correlated with worse overall survival (p=0.036). In stark contrast, highest level of FOLR2 signature correlated with increased overall survival (p=0.0013)(FIG. 4C). The association between the FOLR2 gene-signature and patient clinical outcome was confirmed in an independent BC patient cohort using the same cut-off (Wang et al., 2005a)(FIG. 4D). The association of FOLR2 protein and patient prognosis was also analyzed within 49 ER+/HER2″ patients from the CPTAC dataset. It was found that FOLR2 protein abundance positively correlated with better survival (FIG. 4C). FOLR2 high signature breast cancer patients have a prolonged time to relapse. The FOLR2 signature is a prognostic factor independent of CD8 status in ER+ patients.

Patients treated with endocrine therapy were powerfully stratified by the FOLR2 gene-signature, whereas this was not the case in patients treated with other therapeutic regimens containing chemotherapy or neither chemotherapy nor endocrine therapy. Interestingly, ER/PR+ patients with high FOLR2 gene-signatures had improved survival from endocrine therapy compared to chemotherapy alone, or chemotherapy plus endocrine therapy which was not the case in FOLR2 gene-signature low patients (FIG. 4E). As such, FOLR2 high patients would benefit from endocrine therapy alone and do not require chemotherapy. While FOLR2 low patients could benefit from combined endocrine and chemotherapy.

In the TCGA database: FOLR2 signature as a continuous variable correlates positively with a better outcome in at least 6 other cancer types: KIRC: Kidney Renal Cell Carcinoma; LUAD: Lung adenocarcinoma; LIHC: Liver hepatocellular carcinoma; CESC: cervical squamous cell carcinoma and endocervical adenocarcinoma; MSKCM: Melanoma; and ACC: Adenocortical carcinoma.

Since FOLR2⁺ macrophages are a tissue-resident population in healthy mammary gland, FOLR2 mRNA abundance could be associated to smaller and less aggressive tumors. To test whether this could be a confounding factor in the univariate survival analysis, the level of expression of FOLR2 mRNA was analyzed for breast tumors of different stages and grade. It was found no significant differences in FOLR2 expression between grades and a slight increase in late stage tumors (FIG. 5A). Moreover, multivariate analysis of the prognostic value of the FOLR2 gene-signature adjusted for age, histological grade, tumor size, histology and number of disease-positive LNs, showed that the FOLR2 gene-signature was an independent prognostic factor correlating with better survival of luminal breast cancer patients (FIG. 5B).

It was next wanted to directly assess if the density of FOLR2⁺ macrophages was associated with favorable clinical outcomes. To this end, multispectral imaging were used to analyze a tissue microarray comprising tumors from a retrospective cohort of 122 breast cancer patients. The tumors were stained for FOLR2, cytokeratin and DAPI and calculated the cellular density of FOLR2⁺ macrophages. Using the best performing threshold as a cut-off, it was found that FOLR2⁺ macrophage density positively correlates with patient survival (FIG. 4F). This result was confirmed in a second independent cohort comprising 126 breast cancer patients (FIG. 4F). Altogether these results show that FOLR2⁺ macrophage-abundance is associated with better prognosis for breast cancer patients. In addition, in patients with low CD8+ T cell infiltration, FOLR2+ cell high density also correlates with better survival probability. This identifies the density of FOLR2+ cells as a prognostic value independently of CD8+ T cell density.

5. FOLR2⁺ Macrophages are Spatially Segregated from TREM2⁺ Macrophages Across Cancers.

TREM2⁺ TAMs have been shown to infiltrate tumor nest across cancers (Molgora et al., 2020). It was shown that FOLR2⁺ macrophages are mammary-gland TRMs. Moreover, others have recently shown that macrophages expressing FOLR2 are found in healthy human tissues (Samaniego et al., 2014) (Sharma et al., 2020)(Thomas et al., 2021). Therefore, it was wondered whether FOLR2⁺ macrophages could be detected across cancer types. To address this question, FOLR2 expression was analyzed in 80 histological sections of primary and metastatic tumors across distinct cancers (oral cavity, liver, bladder, brain, kidney, skin, colon, lung, ovary, stomach, breast) FOLR2⁺ macrophages were found across all these cancers. Co-staining for FOLR2 and TREM2 confirmed mutually exclusive expression of the two markers on distinct cells. Staining of FOLR2 and TREM2 on serial sections of various cancer types showed that FOLR2⁺ and TREM2⁺ macrophages are spatially segregated. As described by Molgora et al, TREM2⁺ macrophages infiltrated the tumor nest. By contrast, FOLR2⁺ macrophages were consistently found within peri-tumoral stromal areas. This spatial segregation was found in various cancer types including melanoma, lung carcinoma, hepatocellular carcinoma, oral cavity squamous cell carcinoma, and pancreatic carcinoma. Altogether, these results show that FOLR2⁺ and TREM2⁺ macrophages are associated to specific location within the TME in various cancer types.

The prognostic value of the FOLR2 gene alone or the FOLR2 gene-signature mRNA levels were tested in the TCGA whole tumor transcriptome dataset. It was found that high FOLR2 expression (or FOLR2 gene signature expression), associates with better survival in multiple cancer types including:
Thyroid carcinoma (THCA) (HR=0.527; logRank p=0.005)
Brain lower grade glioma (LGG) (HR=0.7; logRank p=2.7×10⁻⁷)
Adrenocortical carcinoma (ACC) (HR=0.776; logRank p=0.005)
Cervical squamous cell carcinoma and endocervical adenocarcinoma (CESC) (HR=0.849; logRank p=0.025)
lung adenocarcinoma (LUAD) (HR=0.9; logRank p=0.031)
Skin Cutaneous Melanoma (MSKCM) (HR=0.912; logRank p=0.043).
   In addition, the FOLR2 gene signature (but not FOLR2 expression alone) associates with better survival in:
KIRC: Kidney Renal Clear Cell Carcinoma
LIHC: Liver Hepatocellular Carcinoma.
   6. FOLR2⁺ Macrophages are Enriched in CD8⁺ T Cells Infiltrated-Tumors and Co-Localize with Lymphoid Aggregates Across Cancers.

To gain further functional insight, the previously defined FOLR2 gene signature (FOLR2/SLC40A1/SEPP1) or FOLR2 expression was next used alone to correlate abundance of FOLR2⁺ macrophages with other immune and stromal cell types in the TME (FIG. 6A). It was found that the FOLR2 gene signature (or FOLR2 expression alone) positively correlated with known players of anti-tumor immunity like CD8⁺ T cells, DCs, B cells and tertiary lymphoid structures (FIG. 6A). In contrast CADM1⁺TREM2 gene signature (TREM2/SPP1) or TREM2 expression alone did not significantly correlate with T cells, CD8⁺ T cells, NK or B cells (FIG. 6A). In addition, the highest level of FOLR2 expression in bulk tumor transcriptomes coincides, in the same tumor, with coordinated infiltration of multiple lymphocyte lineages and a gene signature for tertiary lymphoid structures (FIG. 6B). By contrast, no correlation was found when patients were stratified according to levels of TREM2 transcript (FIG. 6B). Gene pathways represented in all genes positively correlated to FOLR2 (or TREM2) expression in whole tumor transcriptome were analyzed. In support of previous findings of the inventors, a strong correlation between FOLR2 expression in tumors and various immune pathways was found, including TCR signaling, antigen processing and PD-1 signaling. Gene pathways enriched in FOLR2⁺ TAMs as compared to CADM1⁺TREM2⁺ TAMs were also analyzed in this bulk RNAseq dataset. It was found that gene pathways enriched in FOLR2⁺ TAMs were involved in chemotaxis and functional modules of immune response regulation. Altogether, these results suggest that FOLR2⁺ macrophages are part of an immune contexture underlying the onset of anti-tumor immunity.

Since CD8⁺ T cells have been shown to be associated to better survival in various cancer types including breast cancer (DeNardo et al., 2011)(Ali et al., 2014)(Pagès et al., 2018), it was investigated whether FOLR2⁺ macrophages could be found interacting with tumor-infiltrating CD8⁺ T cells. Using confocal microscopy on tumor resection samples, it was found that FOLR2⁺ macrophages located near CD31⁺ vessels were closely associated with CD8⁺ T cell aggregates. To confirm the spatial association between FOLR2⁺ macrophages and CD8⁺ T cells the previous tissue microarray patient cohort were stained with both CD8 and FOLR2 and calculated their respective cellular density. FOLR2 macrophages^{high} tumor lesions had significantly higher CD8⁺ T cell density than FOLR2 macrophages^{low} lesions (FIG. 6C). Importantly, FOLR2⁺ macrophages found within peri-tumoral stroma were repeatedly enriched in lymphoid aggregates across various cancer types and could also be detected within tertiary lymphoid structures. Altogether, these results show that stroma-associated FOLR2⁺ macrophages are conserved across cancers and are structural component of lymphoid structures near tumor nests. These lymphoid structures are likely to be associated to ongoing immune response.

To further investigate whether FOLR2⁺ macrophages productively engage with CD8⁺ T cells a confocal live imaging was performed on fresh human BC lesions. endogenous CD8⁺ T cells, FOLR2⁺ macrophages and EPCAM⁺ tumor cells from the tumor lesion were stained with fluorescently coupled antibodies against CD8, FOLR2 and EPCAM and the cellular dynamics were subsequently imaged by time-lapse microscopy (FIG. 6D). It was observed that FOLR2⁺ macrophages localized within the tumor stroma and formed a network of sessile cells with active membrane ruffling. Quantification of the speed of displacement of CD8⁺ T cells showed a heterogeneous behavior with more or less motile cells. Importantly, it was reproducibly found that CD8⁺ T cells reduced their speed and established long-lasting contacts with FOLR2⁺ macrophages. This was in contrast with a higher motility of CD8⁺ T cells in FOLR2-deprived tumor regions. It was concluded that CD8⁺ T cells establish prolonged interactions with FOLR2⁺ macrophages, a behavior likely to promote T cell activation. In line with this result, FOLR2 expression in whole tumor transcriptome positively correlated with genes controlling cytotoxic function in T cells (GZMA, GZMB, GZMK, PFR1, KLRB1, KLRD1) but not with genes of T cell dysfunction like LAG3. TREM2 expression showed no significant correlation with genes controlling the cytotoxic function of CD8⁺ T cells.

It has been previously proposed that TAMs in the tumor stroma of lung or in pleural and peritoneal cavities sequester T cells from reaching the tumors and may have a negative impact on anti-tumor immunity (Peranzoni et al., 2018)(Chow et al., Cancer Cell, 2021, 39, 973-988). In other studies, long-lasting interactions between antigen presenting cells and T cells, precede T cell activation and may therefore promote T cell immunity (Hugues et al., Nat. Immunol., 2004, 5, 1235-1242)(Mempel et al., Nature, 2004, 427, 154-159). To assess whether CD8⁺ T cell/macrophages interactions translate into favorable clinical outcome in BC a quantitative spatial analysis of the cellular interactions between CD8⁺ T cells and macrophage subsets was performed in T cell-infiltrated tumors. To this end, multiplex imaging of tumors from ˜60 BC patients in which the density of CD8⁺ T cells was superior to 50 CD8⁺ T cells per mm², was performed. the percentage of CD8⁺ T cells in close proximity (<30 μm) with either FOLR2⁺ or TREM2⁺ macrophages was quantified and the patients were stratified according to the percentage “high” or “low” of CD8⁺ T cells in close contact with these two macrophage subsets. It was found that a high percentage of CD8⁺ T cells in close contact with FOLR2⁺ macrophages associated with a better outcome for BC patients, while this was not the case for CD8⁺ T cells interacting with TREM2⁺ macrophages. It was therefore concluded that tumor infiltrating CD8⁺ T cells actively engage with FOLR2⁺ macrophages and that this interaction positively correlates with CD8⁺ T cell activation and patient survival. It was therefore concluded that FOLR2⁺ macrophages represent a defined macrophage population associated to the onset of anti-tumoral immune responses.

### 7. FOLR2+ Macrophages can Promote T Cell Effector Differentiation

Nascent tumors have been shown to engage in cellular crosstalk with TRMs which in turn promote tumor growth, motility and invasiveness (Cassetta and Pollard, 2018). The PyMT BC murine model was used to explore the functional characteristics of FOLR2⁺ macrophages. First, it was asked whether FOLR2⁺ macrophages of healthy mammary gland would respond to the developing tumor. To this end, bulk RNAseq was used to profile FOLR2⁺ macrophages isolated from healthy versus small or medium tumor murine mammary glands. Principal component (PC) analysis using the 10,000 most variable genes showed that FOLR2⁺ macrophages isolated from mammary tumors changed their transcriptomic profile in a tumor-size dependent manner as seen by the variations in the PC2 axis. 6,139 DEG were found between FOLR2⁺ and CADM1⁺ macrophages isolated from the same tumors. It was concluded that FOLR2⁺ macrophages respond to the developing tumor but remain separable cellular entities from CADM1⁺ macrophages. 1.356 DEG were found between mammary tumor FOLR2⁺ macrophages and healthy mammary gland FOLR2⁺ macrophages (FIG. 7A). Among these DEG, mammary tumor FOLR2⁺ macrophages expressed genes involved in the positive regulation of immune system processes including B and T cell chemoattractants (Ccl6 to 9, Ccl12, Cxcl2, Cxcl13, Cxcl14, Cxcl16); adhesion molecules (Icam1, Vcam1, Fn1) and lysosomal proteins (Ctse, Rab32). In contrast, FOLR2⁺ macrophages isolated from healthy mammary glands were enriched in genes regulating metabolic processes (Igf1, Srebf2, Abcd2). Therefore, these data show that FOLR2⁺ TRMs respond to tumor development.

Macrophage activation in tumors is often referred as “pro-inflammatory/M1” versus “anti-inflammatory/M2” (Mantovani et al., 2002)(Murray et al., Immunity, 2014, 41, 14-20). To test whether mammary tumor FOLR2⁺ and CADM1⁺ macrophage subsets harbor such functional specialization, the expression of genes defining M1 or M2 gene-signatures were analyzed in the two macrophage subsets (Azizi et al., 2018). It was found that both mammary tumor FOLR2⁺ and CADM1⁺ macrophages concomitantly express individual M1 and M2 genes. For example, FOLR2⁺ macrophages expressed Cd80, Cd40, and Il6 “M1 genes” and Cd163, Mrc1, Il10 “M2 genes”. CADM1⁺ macrophages expressed Cd86, Cxcl9, Il12b “M1 genes” and Vegfa, Cd276, Tgfb3 “M2 genes”. This shows that macrophage activation in the tumor microenvironment does not fit with the in vitro M1/M2 polarization model and reveals the complexity of macrophage activation in the tumor microenvironment. Interestingly, mammary tumor FOLR2⁺ and CADM1⁺ macrophage subsets expressed distinct sets of functional genes that could be linked to T cell activation. Therefore, to directly test the T cell-activation potential of the macrophage subsets two assays were set up using FOLR2⁺ and CADM1⁺ macrophages isolated from the same tumors and co-cultured with CD8⁺ T cells. First, a T cell suppression assay was set up in which purified TAMs were co-cultured with polyclonal activated CD8⁺ T cells. Here, FOLR2⁺ macrophage did not display suppressive activity. Instead, FOLR2⁺ macrophage improved CD8⁺T cell proliferation and differentiation (loss of CD62L and upregulation of CD44 and CD25). CADM1⁺ macrophages did not suppress CD8⁺T cell activation either but their ability to promote effector T cell differentiation was weaker than FOLR2⁺ macrophages. In previous studies (Ruffell et al., Cancer Cell, 2014, 26, 623-627)(Katzenelenbogen et al., Cell, 2020, 182, 872-885) immunosuppressive activity was restricted to MHCII^{NEG} tumor associated myeloid cells. Instead, MHCII⁺ TAMs were not exerting T cell-inhibition. It is shown here that both CADM1⁺ and FOLR2⁺ TAMs express high levels of MHCII consistent with their lack of immunosuppressive activity. Since neither FOLR2⁺ and CADM1⁺ macrophage strongly suppressed T cell expansion, an antigen-specific T cell priming assay was set up. Purified FOLR2⁺ and CADM1⁺ macrophages were loaded with the OTI specific SIINFEKL peptide, washed and subsequently co-cultured with naïve OTI CD8⁺ T cells. In comparison to CADM1⁺ macrophages isolated from the same tumors, FOLR2⁺ macrophages showed higher capacity to induce the activation of naïve T cells, their expansion, their polyfunctionality (IL-2, IFN-γ, TNF-α) and cytotoxic function (expression of granzyme B) (FIG. 7B). Furthermore, it was found that FOLR2⁺ macrophages isolated from healthy mammary glands did not efficiently activate OTI CD8⁺ T cells, while FOLR2⁺ macrophages isolated from mammary tumors could induce T cell expansion and differentiation (FIG. 7B). These results confirm that FOLR2⁺ macrophages are activated during tumor development and acquire the ability to prime CD8⁺ T cells. In sum, these results provide evidence that FOLR2⁺ macrophages do not behave like immunosuppressive cells. Instead, tumor-associated FOLR2⁺ macrophages are potent antigen presenting cells displaying the functional ability to trigger CD8⁺ T cell-activation.

FOLR2 macrophages were purified from mouse mammary tumors and loaded with OVA peptide. After washing, FOLR2 macrophages were co-cultured for 3 days with anti-OVA naive CD8 T cells. At day 3 after culture, activation and proliferation of T cell was measured. FOLR2 macrophage show higher capacity to activate T cells than CADM1 macrophages or OVA alone (FIG. 7C).

8. Vaccination using Anti-FOLR2 Targeting Antibody Coupled to a Model Antigen Elicit a Specific CD8+ T Cell Response

To deliver tumor antigens to FOLR2⁺ macrophages, an anti-FOLR2 antibody linked to the CD8⁺ T cell-specific OVA peptide (SIINFEKL) was generated (FIG. 8). Preliminary data show that immunization with anti-FOLR2-SIINFEKL induces the activation of adoptively transferred OVA-specific OTI CD8⁺ T cells validating the feasibility of the approach (FIG. 8).

### Discussion

In order to understand the phenotypic and functional heterogeneity of macrophage populations in human breast cancers, a single-cell atlas of myeloid cells infiltrating human luminal breast tumors was generated. Two phenotypically distinct TAM subsets were identified: TREM2⁺CADM1⁺ macrophages and FOLR2⁺ macrophages. In stark contrast with TREM2⁺ macrophage whose infiltration has been previously associated to worst clinical outcomes (Molgora et al., 2020), it is shown that the abundance of FOLR2⁺ TAMs is predictive of better clinical outcomes. In addition, it is established that FOLR2⁺ TAMs are spatially associated with sites of lymphocyte infiltration within the TME across cancers.

FOLR2⁺ Macrophages: a Subset of TRMs Associated with Favorable Clinical Outcome.

In this study, an evolutionarily conserved tissue-resident FOLR2⁺ macrophage population present in both human and mouse healthy mammary gland was identified In mice, breast TRMs regulate post-natal mammary gland development and remodeling (Gouon-Evans et al., 2002)(Van Nguyen and Pollard, 2002). Human FOLR2⁺ macrophages express MRC1 and LYVE1 and align to murine MRC1⁺ TRMs that had been described in adult mammary gland of healthy nulliparous mice (Jäppinen et al., 2019)(Wang et al., 2020). MRC1⁺ TRMs arise from fetal precursors as demonstrated by genetic labeling at E8.5 or E13.5 in fate-mappers Csf1r^{Mer-iCre-Mer} or Cx3cr1^{Cre-ERT2} mice respectively (Jäppinen et al., 2019). In addition, MRC1⁺ TRMs exhibit a self-renewing capability (Wang et al., 2020). MRC1⁺ TRMs have a non-redundant function in mammary gland development: inhibition of MRC1⁺ TRM development in Plvap^{-/-} mice significantly impairs ductal morphogenesis during puberty (Jäppinen et al., 2019). In mice, MRC1⁺ TRMs co-exist with a minor fraction of CX3CR1^{high}MRC1⁻ TRMs endowed with an intra-ductal localization (Dawson et al., 2020). Intra-ductal CX3CR1^{high}MRC1⁻ macrophages develop from adult monocytes and they expand during tissue remodeling imposed by lactation (Dawson et al., 2020). Interestingly, most breast tumor-invading murine TAMs align transcriptionally to homeostatic intra-ductal CX3CR1^{high}MRC1⁻ TRM (Dawson et al., 2020). In sum, these findings highlight the ontogenetic and functional diversity within mammary gland TRM subsets (Ginhoux and Guilliams, 2016).

Here the FOLR2⁺ macrophages were identified as human orthologs of murine MRC1⁺ breast TRMs. It was found that human FOLR2⁺ macrophages represent the main macrophage population in healthy breast tissue. This defines FOLR2⁺ macrophages as bona fide mammary gland-resident macrophages. In contrast, it is shown that CADM1⁺TREM2⁺ macrophages are scarce in healthy tissue and increase in metastatic LN and primary tumors. It is shown that human CADM1⁺TREM2⁺ macrophages align with murine CX3CR1^{high}MRC1⁻ TAMs. These findings are consistent with a recent study identifying TREM2⁺ macrophages in multiple cancer types (Molgora et al., 2020). The infiltration of CADM1⁺TREM2⁺ macrophages is likely to rely on influx of circulating monocytes locally attracted to tumor lesions. In support of this view, this bulk RNAseq analysis of human CADM1⁺TREM2⁺ macrophages demonstrate their transcriptional closeness to CCR2⁺ monocytes.

Do TRMs have a specific function during carcinogenesis? Recent studies have reported pro- tumorigenic activities for murine TRMs. For instance, depletion of embryonic-derived pro-fibrotic TRMs delays the progression of tumor lesions in pancreatic ductal adenocarcinoma murine models (Zhu et al., 2017). However, it is not known if this impacts on overall survival. Also, depletion of CD163⁺ TRMs in ovarian cancer reduces epithelial to mesenchymal transition and overall tumor growth (Etzerodt et al., 2020). In murine PyMT breast cancer, Franklin et al. have shown that MRC1⁺ TRMs present in healthy mammary glands, persist in developing murine breast adenocarcinoma despite dilution by incoming monocyte-derived TAMs (Franklin et al., 2014). The pro-tumorigenic function of TRMs found in pancreatic and ovarian cancers does not seem to apply to breast cancer in which MRC1⁺ TRMs are less immunosuppressive than monocyte-derived, NOTCH-dependent TAMs (Franklin et al., 2014)(Kitamura et al., 2018). MRC1⁺ TRM depletion prior to carcinogenesis did not affect tumor growth in autochthonous MMTV-PyMT or MMTV-Her2 mouse models (Franklin et al., 2014)(Linde et al., 2018) despite an effect on early cancer cell dissemination (Linde et al., 2018).

In human, the lack of specific markers distinguishing monocyte-derived TAMs versus TRMs has so far precluded an assessment of their function. To address this gap in knowledge, specific gene-signatures and surface markers have been defined enabling careful identification of macrophage subsets within tumors. To probe the association of FOLR2⁺ macrophages with clinical outcome in breast cancer a gene-signature was designed enabling to infer FOLR2⁺ macrophages-abundance in breast cancer bulk transcriptomes. It was found that the abundance of FOLR2⁺ macrophages, but not of CADM1⁺TREM2⁺ macrophages, associated with better prognosis. FOLR2 signature is predictive of better survival in at least six other cancers. A validation of this in silico finding was provided showing that the density of FOLR2⁺ macrophages in a retrospective cohort of 122 breast cancer patients correlates with increased survival. Altogether, these results establish FOLR2⁺ macrophages as a biomarker of favorable outcome.

### Functional Diversity of Perivascular Macrophages.

It was observed that FOLR2⁺ macrophages present a transcriptional signature of steady state perivascular (PV) macrophages (LYVE1, MRC1, TIMD4, MAF). Accordingly, it was found that some FOLR2⁺ macrophages located in close proximity to CD31⁺ vessels. In mice, various studies have identified PV macrophages across organs including lung and skin (Chakarov et al., 2019), brain (Goldmann et al., 2016)(Sg et al., 2020), arterial wall (Lim et al., 2018), mammary gland (Jäppinen et al., 2019) and spleen (Mebius and Kraal, 2005). However, less is known about the phenotype of human PV macrophages (Lapenna et al., 2018).

In mouse models, a pro-tumoral subset of PV macrophages expressing the angiopoietin receptor TIE2 has been implicated in angiogenesis and tumor spreading (Lewis et al., 2016)(De Palma et al., 2005)(Pucci et al., 2009). Mechanistically, TIE2⁺ PV macrophages release VEGFA that favors tumor cell intravasation and metastasis by reducing tight-junctions in tumor blood vessels promoting permeabilization of the vascular wall (Harney et al., 2015). TIE2⁺ PV macrophages respond to endothelial-derived angiopoietin 2 (ANG2) engaging the TIE2 receptor thereby supporting PV positioning and pro-angiogenic function (Mazzieri et al., 2011). It is unclear if FOLR2⁺ PV macrophages described in this study align to TIE2⁺ PV macrophages. This hypothesis was not favored for two reasons. First, it was not possible to document TIE2 expression in single cell- and bulk-RNAseq analysis of FOLR2⁺ macrophages. Second, TIE2⁺ PV macrophages ontogeny relies on the progressive infiltration of a specialized subset of pro-angiogenic TIE2⁺ monocytes (De Palma et al., 2005)(Pucci et al., 2009)(Coffelt et al., 2010)(Arwert et al., 2018). This contrasts with these data evidencing FOLR2⁺ macrophages are TRMs. Therefore, further experimental efforts are needed to disentangle the heterogeneity of PV macrophages.

### A Role for FOLR2⁺ Macrophages in Promoting Lymphocyte Infiltration and Anti-Tumor Immunity

A recent study has identified a subset of FOLR2⁺ TRMs associated to human hepatocellular carcinoma and resembling fetal-liver macrophages (Sharma et al., 2020). CD86 expression in hepatic FOLR2⁺ macrophages suggest that they might be interacting with CTLA4⁺ regulatory T cells. To probe the function of FOLR2⁺ macrophages in breast cancer, this gene signature was used to infer FOLR2⁺ macrophages-abundance in bulk tumor microarray and correlate it to the infiltration of other immune cells. It was found that the infiltration of FOLR2⁺ macrophages, in contrast with TREM2⁺ macrophages infiltration, positively correlated with tumor-infiltrating lymphocytes including CD8⁺ T cell, B cells as well as DCs. In addition, it was found by confocal imaging that FOLR2⁺ macrophages co-localized with CD8⁺ T cell aggregates in the vicinity of endothelial cells. The correlation between FOLR2 and CD8⁺ T cell abundance was validated by multispectral imaging: tumor lesions highly infiltrated with FOLR2⁺ macrophages had significantly higher CD8⁺ T cell-density. Since CD8⁺ T cell infiltration correlates with better survival probability in many cancers including breast cancer (DeNardo et al., 2011)(Ali et al., 2014), these results suggest that FOLR2⁺ macrophages participate to the onset of anti-tumor immunity. FOLR2⁺ macrophages have been described in human tissues including fetal liver, placenta, colon (Samaniego et al., 2014)(Sharma et al., 2020)(Thomas et al., 2021). Here these observations were extended in healthy mammary gland and breast cancer and multiple other cancer types. Importantly, the association of FOLR2⁺ macrophages with peri-tumoral, stromal lymphoid aggregates is found in multiple cancer types. In some instances, FOLR2⁺ macrophages infiltrating organized tertiary lymphoid structures comprising B and T cells are reported here. Murine PV macrophages have been described as possible regulators of lymphocyte infiltration during inflammation (Natsuaki et al., 2014) and auto-immunity (Mohan et al., 2017). It can be hypothesized that FOLR2⁺ macrophages could regulate the infiltration of CD8⁺T cells by different mechanisms: directly by delivering chemokines attracting T cells (Dangaj et al., 2019), or indirectly by delivering inflammatory cytokines to endothelial cells or growth factor to pericytes (Minutti et al., 2019). Further studies are needed to identify the mechanisms by which FOLR2⁺ macrophages regulate lymphocyte infiltration in tumors, a key event for the development of efficient anti-tumor immune responses. This study highlights antagonistic roles for tumor-associated macrophage subsets and paves the way for subset-specific therapeutic interventions in macrophages-based cancer therapies.

TABLE
List of antibodies used in the study

| | | | |
|---|---|---|---|
| Name | Fluorochrome | Source | Identifier |

anti-human antibodies for immunohistochemistry and immunofluorescence

| | | | |
|---|---|---|---|
| CD3 | N/A | Dako | Cat# A0452 |
| APOE | N/A | Abcam | Cat# ab52607 |
| CD11c | N/A | Protein tech | Cat# 60258 |
| AE1/AE3 (Cytokeratin) | N/A | Dako | Cat# M0851 |
| LYVE-1 | N/A | Milipore | Cat# MABS129 |
| CD31 | N/A | Dako | Cat# M082 |
| FOLR2 | APC | Biolegend | Cat# 391705 |
| CD206 | PE | BD Biosciences | Cat# 555954 |
| CD8 | PerCP/Cy5.5 | BD Biosciences | Cat# 565310 |
| CD31 | AlexaFluor 488 | Biolegend | Cat# 303109 |
| EpCAM (CD326) | BrilhantViolet421 | Biolegend | Cat# 324219 |
| CD8 | N/A | Dako | Cat# GA623 |
| FOLR2 | N/A | Thermo Fischer Scientific | Cat# MA5-26933 |
| AE1/AE3 (Cytokeratin) | N/A | Abcam | Cat# ab86734 |
| FOLR2 | PE | Biolegend | Cat# 391703 |

anti-human antibodies or reagent for flow cytometry

| | | | |
|---|---|---|---|
| FOLR2 | PE | Biolegend | Cat# 391703 |
| FOLR2 | APC | Biolegend | Cat# 391705 |
| Pan cancer | BUV395 | BD Biosciences | Cat# 745704 |
| EpCAM | eFluor660 | eBioscience | Cat# 50-9326-42 |
| CD45 | APC-Cy7 | BD Biosciences | Cat# 557833 |
| CD11c | PE-Cy7 | Thermo Fischer Scientific | Cat# 25-0116-42 |
| HI-ADR | Percp-Cy5.5 | BD Biosciences | Cat# 552764 |
| CD1c | PerCP-eFluor 710 | Thermo Fischer Scientific | Cat# 46-0015-42 |
| CD14 | AlexaFluor 700 | BD Biosciences | Cat# 557923 |
| CD14 | FITC | eBioscience | Cat# 11-0149-42 |
| CD163 | Brilhant Violet 605 | BD Biosciences | Cat# 745091 |
| CD204 (MRS1) | Brilhant Violet 786 | BD Biosciences | Cat# 742443 |
| S100A8/9 | AlexaFluor 647 | BD Biosciences | Cat# 566010 |
| APOE | AlexaFluor 488 | Abcam | Cat# ab196463 |
| CCR2 | Brilhant Violet 421 | Biolegend | Cat# 357210 |
| CCR2 | PE/Dazzle 594 | Biolegend | Cat# 357222 |
| Anti-SynCAM(TSLC1/CADM1) | N/A | MBL | Cat# CM004-3 |
| CD64 | PerCP-eFluor 710 | eBioscience | Cat# 46-0649-42 |
| CD64 | PE-CF594 | BD Biosciences | Cat# 565389 |
| CD3 | APC | BD Biosciences | Cat# 561810 |
| CD3 | Brilhant Violet 650 | BD Biosciences | Cat# 563851 |
| CD19 | APC | BD Biosciences | Cat# 555415 |
| CD19 | Brilhant Violet 650 | BD Biosciences | Cat# 563226 |
| CD26 | Brilhant Violet 650 | BD Biosciences | Cat# 744451 |
| BTLA | Brilhant Violet 650 | BD Biosciences | Cat# 564803 |
| XCR1 | FITC | Biolegend | Cat# 372612 |
| Live and Dead | AQUA | Thermo Fischer Scientific | Cat# L34957 |
| FcR blocking reagent | N/A | Miltenyi | Cat# 130-059-901 |

human antibodies for mass cytometry
anti-mouse antibodies for flow cytometry

| | | | |
|---|---|---|---|
| Donkey Anti-Chicken secondary | AlexaFluor594 | JacksonImmunoResearch | Cat# 703-586-155 |
| NKp64 | FITC | eBioscience | Cat# 11-3351-82 |
| B220 | FITC | BD | Cat# 553088 |
| CD19 | FITC | Biolegend | Cat# 101506 |
| CD3E | FITC | Biolegend | Cat# 100306 |
| Ly6G | FITC | BD | Cat# 551460 |
| CD64 | APC | Biolegend | Cat# 139306 |
| CD45 | APC-Cy7 | BD | Cat# 557659 |
| MHCII (I-A I-E) | AF700 | eBioscience | Cat# 56-5321-82 |
| CCR2 | BV421 | Biolegend | Cat# 150605 |
| F4/80 | BV605 | Biolegend | Cat# 123133 |
| CD11b | BV650 | Biolegend | Cat# 101259 |
| Ly6C | BV785 | Biolegend | Cat# 128041 |
| CD11c | PC7 | eBioscience | Cat# 25-0114-82 |
| FOLR2 | PE | Biolegend | Cat# 153303 |

PGP-21,Tl

### REFERENCES

Ali, H. R., Provenzano, E., Dawson, S.-J., Blows, F.M., Liu, B., Shah, M., Earl, H. M., Poole, C. J., Hiller, L., Dunn, J. A., et al. (2014). Association between CD8+ T-cell infiltration and breast cancer survival in 12,439 patients. Ann. Oncol. 25, 1536-1543.
Ali, H. R., Chlon, L., Pharoah, P. D. P., Markowetz, F., and Caldas, C. (2016). Patterns of Immune Infiltration in Breast Cancer and Their Clinical Implications: A Gene-Expression-Based Retrospective Study. PLOS Medicine 13, e1002194.
Anders, S., and Huber, W. (2010). Differential expression analysis for sequence count data. Genome Biol 11, R106.
Arwert, E. N., Harney, A.S., Entenberg. D., Wang, Y., Sahai, E., Pollard, J. W., and Condeelis, J. S. (2018). A Unidirectional Transition from Migratory to Perivascular Macrophage Is Required for Tumor Cell Intravasation. Cell Reports 23, 1239-1248.
Azizi, E., Carr, A. J., Plitas, G., Cornish, A. E., Konopacki, C., Prabhakaran, S., Nainys, J., Wu, K., Kiseliovas, V., Setty, M., et al. (2018). Single-Cell Map of Diverse Immune Phenotypes in the Breast Tumor Microenvironment. Cell.
Binnewies, M., Roberts, E. W., Kersten, K., Chan, V., Fearon, D. F., Merad, M., Coussens, L. M., Gabrilovich, D.I., Ostrand-Rosenberg, S., Hedrick, C. C., et al. (2018). Understanding the tumor immune microenvironment (TIME) for effective therapy. Nature Medicine 24, 541-550.
Bonapace, L., Coissieux, M.-M., Wyckoff, J., Mertz, K. D., Varga, Z., Junt, T., and Bentires-Alj. M. (2014). Cessation of CCL2 inhibition accelerates breast cancer metastasis by promoting angiogenesis. Nature 515, 130-133.
Bourdely, P., Anselmi, G., Vaivode, K., Ramos, R.N., Missolo-Koussou, Y., Hidalgo, S., Tosselo, J., Nuñez, N., Richer, W., Vincent-Salomon, A., et al. (2020). Transcriptional and Functional Analysis of CD1c+ Human Dendritic Cells Identifies a CD163+ Subset Priming CD8+CD103+ T Cells. Immunity. Cassetta, L., and Pollard, J.W. (2018). Targeting macrophages: therapeutic approaches in cancer. Nature Reviews Drug Discovery.
Cassetta, L., Fragkogianni, S., Sims, A. H., Swierczak, A., Forrester, L. M., Zhang, H., Soong, D. Y. H., Cotechini, T., Anur, P., Lin, E. Y., et al. (2019). Human Tumor-Associated Macrophage and Monocyte Transcriptional Landscapes Reveal Cancer-Specific Reprogramming, Biomarkers, and Therapeutic Targets. Cancer Cell.
Caux, C., Ramos, R. N., Prendergast, G. C., Bendriss-Vermare, N., and Ménétrier-Caux, C. (2016). A Milestone Review on How Macrophages Affect Tumor Growth. Cancer Res 76, 6439-6442.
Chakarov, S., Lim, H.Y., Tan, L., Lim, S. Y., Sec, P., Lum, J., Zhang, X.-M., Foo, S., Nakamizo, S., Duan, K., et al. (2019). Two distinct interstitial macrophage populations coexist across tissues in specific subtissular niches. Science 363, eaau0964.
Chen, X., Li, J., Gray, W. H., Lehmann, B. D., Bauer, J. A., Shyr, Y., and Pietenpol, J. A. (2012). TNBCtype: A Subtyping Tool for Triple-Negative Breast Cancer: Cancer Informatics.
Coffelt, S. B., Tal, A.O., Scholz, A., De Palma, M., Patel, S., Urbich, C., Biswas, S. K., Murdoch, C., Plate, K. H., Reiss, Y., et al. (2010). Angiopoietin-2 regulates gene expression in TIE2-expressing monocytes and augments their inherent proangiogenic functions. Cancer Res. 70, 5270-5280.
Colonna, M., Trinchieri, G., and Liu, Y.-J. (2004). Plasmacytoid dendritic cells in immunity. Nat. Immunol 5, 1219-1226.
Dangaj. D., Bruand, M., Grimm, A. J., Ronet, C., Barras, D., Duttagupta, P. A., Lanitis, E., Duraiswamy, J., Tanyi, J. L., Benencia, F., et al. (2019). Cooperation between Constitutive and Inducible Chemokines Enables T Cell Engraftment and Immune Attack in Solid Tumors. Cancer Cell 35, 885-900.e10.
Davie, S. A., Maglione, J. E., Manner, C.K., Young. D., Cardiff, R. D., MacLeod, C. L., and Ellies, L. G. (2007). Effects of FVB/NJ and C57Bl/6J strain backgrounds on mammary tumor phenotype in inducible nitric oxide synthase deficient mice. Transgenic Res. 16, 193-201.
Dawson, C. A., Pal, B., Vaillant, F., Gandolfo, L. C., Liu, Z., Bleriot, C., Ginhoux, F., Smyth, G. K., Lindeman, G. J., Mueller, S. N., et al. (2020). Tissue-resident ductal macrophages survey the mammary epithelium and facilitate tissue remodelling. Nat. Cell Biol. 22, 546-558.
DeNardo, D. G., Brennan, D. J., Rexhepaj, E., Ruffell, B., Shiao, S. L., Madden, S. F., Gallagher, W. M., Wadhwani, N., Keil, S. D., Junaid, S. A., et al. (2011). Leukocyte Complexity Predicts Breast Cancer Survival and Functionally Regulates Response to Chemotherapy. Cancer Discov 1, 54-67.
Dobin, A., Davis, C.A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Dutertre, C.-A., Becht, E., Irac, S. E., Khalilnezhad, A., Narang. V., Khalilnezhad, S., Ng, P. Y., van den Hoogen, L. L., Leong, J. Y., Lee, B., et al. (2019). Single-Cell Analysis of Human Mononuclear Phagocytes Reveals Subset-Defining Markers and Identifies Circulating Inflammatory Dendritic Cells. Immunity 57, 573-589.e8.
Engblom, C., Pfirschke, C., and Pittet, M. J. (2016). The role of myeloid cells in cancer therapies. Nat. Rev. Cancer 16, 447-462.
Etzerodt, A., Moulin, M., Doktor, T.K., Delfini, M., Mossadegh-Keller, N., Bajenoff, M., Sieweke, M. H., Moestrup, S.K., Auphan-Anezin, N., and Lawrence, T. (2020). Tissue-resident macrophages in omentum promote metastatic spread of ovarian cancer. J. Exp. Med. 217.
Franklin, R. A., Liao, W., Sarkar, A., Kim, M. V., Bivona, M. R., Liu, K., Pamer, E. G., and Li, M. O. (2014). The cellular and molecular origin of tumor-associated macrophages. Science 344, 921-925.
Gallina, G., Dolcetti, L., Serafini, P., Santo, C. D., Marigo, I., Colombo, M. P., Basso, G., Brombacher, F., Borrello, I., Zanovello, P., et al. (2006). Tumors induce a subset of inflammatory monocytes with immunosuppressive activity on CD8⁺ T cells. J Clin Invest 116, 2777-2790.
Ginhoux, F., and Guilliams, M. (2016). Tissue-Resident Macrophage Ontogeny and Homeostasis. Immunity 44, 439-449.
Ginhoux, F., Greter, M., Lebocuf, M., Nandi, S., See, P., Gokhan, S., Mehler, M. F., Conway, S. J., Ng. L.G., Stanley, E. R., et al. (2010). Fate Mapping Analysis Reveals That Adult Microglia Derive from Primitive Macrophages. Science 330, 841-845.
Goldmann, T., Wieghofer, P., Jordão, M. J. C., Prutek, F., Hagemeyer, N., Frenzel, K., Amann, L., Staszewski, O., Kierdorf, K., Krueger, M., et al. (2016). Origin, fate and dynamics of macrophages at central nervous system interfaces. Nat Immunol 17, 797-805.
Gouon-Evans, V., Lin, E. Y., and Pollard, J. W. (2002). Requirement of macrophages and eosinophils and their cytokines/chemokines for mammary gland development. Breast Cancer Res 4, 1-10.
Guilliams, M., and Scott, C. L. (2017). Does niche competition determine the origin of tissue-resident macrophages? Nature Reviews Immunology 17, 451-460.
Han, X., Wang. R., Zhou, Y., Fei, L., Sun, H., Lai, S., Saadatpour, A., Zhou, Z., Chen, H., Ye, F., et al. (2018). Mapping the Mouse Cell Atlas by Microwell-Seq. Cell 172, 1091-1107.e17.
Hanna, R. N., Cekic, C., Sag, D., Tacke, R., Thomas, G. D., Nowyhed, H., Herrley, E., Rasquinha, N., McArdle, S., Wu, R., et al. (2015). Patrolling monocytes control tumor metastasis to the lung. Science 350, 985-990.
Harney, A. S., Arwert, E. N., Entenberg, D., Wang, Y., Guo, P., Qian, B.-Z., Oktay, M. H., Pollard, J. W., Jones, J. G., and Condeelis, J. S. (2015). Real-Time Imaging Reveals Local, Transient Vascular Permeability, and Tumor Cell Intravasation Stimulated by TIE2hi Macrophage-Derived VEGFA. Cancer Discov 5, 932-943.
Jäppinen, N., Félix, I., Lokka, E., Tyystjärvi, S., Pynttäri, A., Lahtela, T., Gerke, H., Elima, K., Rantakari, P., and Salmi, M. (2019). Fetal-derived macrophages dominate in adult mammary glands. Nat Commun 10, 281.
Kitamura, T., Doughty-Shenton, D., Cassetta, L., Fragkogianni, S., Brownlie, D., Kato, Y., Carragher, N., and Pollard, J. W. (2018). Monocytes Differentiate to Immune Suppressive Precursors of Metastasis-Associated Macrophages in Mouse Models of Metastatic Breast Cancer. Front. Immunol. 8.
Lapenna, A., De Palma, M., and Lewis, C. E. (2018). Perivascular macrophages in health and disease. Nature Reviews Immunology.
Leek, R. D., Lewis, C. E., Whitehouse, R., Greenall, M., Clarke, J., and Harris, A. L. (1996). Association of Macrophage Infiltration with Angiogenesis and Prognosis in Invasive Breast Carcinoma. Cancer Res 56, 4625-4629.
Leruste, A., Tosello, J., Ramos, R. N., Tauziède-Espariat, A., Brohard, S., Han, Z.-Y., Beccaria, K., Andrianteranagna, M., Caudana, P., Nikolic, J., et al. (2019). Clonally Expanded T Cells Reveal Immunogenicity of Rhabdoid Tumors. Cancer Cell S1535610819304829.
Lewis, C. E., and Pollard, J. W. (2006). Distinct Role of Macrophages in Different Tumor Microenvironments. Cancer Res 66, 605-612.
Lewis, C. E., Harney, A. S., and Pollard, J. W. (2016). The Multifaceted Role of Perivascular Macrophages in Tumors. Cancer Cell 30, 18-25.
Lim, H. Y., Lim, S. Y., Tan, C. K., Thiam, C. H., Goh, C. C., Carbajo, D., Chew, S. H. S., See, P., Chakarov, S., Wang, X. N., et al. (2018). Hyaluronan Receptor LYVE-1-Expressing Macrophages Maintain Arterial Tone through Hyaluronan-Mediated Regulation of Smooth Muscle Cell Collagen. Immunity 49, 326-341.e7.
Lin, E. Y., Jones, J. G., Li, P., Zhu, L., Whitney, K. D., Muller, W. J., and Pollard, J. W. (2003). Progression to Malignancy in the Polyoma Middle T Oncoprotein Mouse Breast Cancer Model Provides a Reliable Model for Human Diseases. The American Journal of Pathology 163, 2113-2126.
Lin, E. Y., Li, J.-F., Gnatovskiy, L., Deng, Y., Zhu, L., Grzesik, D. A., Qian, H., Xue, X.-n., and Pollard, J. W. (2006). Macrophages Regulate the Angiogenic Switch in a Mouse Model of Breast Cancer. Cancer Research 66, 11238-11246.
Linde, N., Casanova-Acebes, M., Sosa, M. S., Mortha, A., Rahman, A., Farias, E., Harper, K., Tardio, E., Reyes Torres, I., Jones, J., et al. (2018). Macrophages orchestrate breast cancer early dissemination and metastasis. Nat Commun 9.
Love, M. I., Huber, W., and Anders, S. (2014). Moderated estimation of fold change and dispersion for RNA-seq data with DESeq2. Genome Biol 15, 1-21.
Loyher, P.-L., Hamon, P., Laviron, M., Meghraoui-Kheddar, A., Goncalves, E., Deng, Z., Torstensson, S., Bercovici, N., Baudesson de Chanville, C., Combadière, B., et al. (2018). Macrophages of distinct origins contribute to tumor development in the lung. The Journal of Experimental Medicine jem.20180534.
Mantovani, A., Sozzani, S., Locati, M., Allavena, P., and Sica, A. (2002). Macrophage polarization: tumor-associated macrophages as a paradigm for polarized M2 mononuclear phagocytes. Trends in Immunology 23, 549-555.
Mantovani, A., Sica, A., Sozzani, S., Allavena, P., Vecchi, A., and Locati, M. (2004). The chemokine system in diverse forms of macrophage activation and polarization. Trends in Immunology 25, 677-686.
Mantovani, A., Marchesi, F., Malesci, A., Laghi, L., and Allavena, P. (2017). Tumour-associated macrophages as treatment targets in oncology. Nature Reviews Clinical Oncology 14,399-416.
Mazzieri, R., Pucci, F., Moi, D., Zonari, E., Ranghetti, A., Berti, A., Politi, L.S., Gentner, B., Brown, J. L., Naldini, L., et al. (2011). Targeting the ANG2/TIE2 Axis Inhibits Tumor Growth and Metastasis by Impairing Angiogenesis and Disabling Rebounds of Proangiogenic Myeloid Cells. Cancer Cell 19, 512-526.
Mebius, R. E., and Kraal, G. (2005). Structure and function of the spleen. Nat. Rev. Immunol. 5, 606-616.
METABRIC Group, Curtis, C., Shah, S.P., Chin, S.-F., Turashvili, G., Rueda, O. M., Dunning, M. J., Speed, D., Lynch, A. G., Samarajiwa, S., et al. (2012). The genomic and transcriptomic architecture of 2,000 breast tumours reveals novel subgroups. Nature 486, 346-352.
Minutti, C. M., Modak, R.V., Macdonald, F., Li, F., Smyth, D. J., Dorward, D. A., Blair, N., Husovsky, C., Muir, A., Giampazolias, E., et al. (2019). A Macrophage-Pericyte Axis Directs Tissue Restoration via Amphiregulin-Induced Transforming Growth Factor Beta Activation. Immunity 50, 645-654.e6.
Mohan, J. F., Kohler, R. H., Hill, J. A., Weissleder, R., Mathis, D., and Benoist, C. (2017). Imaging the emergence and natural progression of spontaneous autoimmune diabetes. Proc Natl Acad Sci USA 114, E7776-E7785.
Molgora, M., Esaulova, E., Vermi, W., Hou, J., Chen, Y., Luo, J., Brioschi, S., Bugatti, M., Omodei, A. S., Ricci, B., et al. (2020). TREM2 Modulation Remodels the Tumor Myeloid Landscape Enhancing Anti-PD-1 Immunotherapy. Cell 182, 886-900.e17.
Natsuaki, Y., Egawa, G., Nakamizo, S., Ono, S., Hanakawa, S., Okada, T., Kusuba, N., Otsuka, A., Kitoh, A., Honda, T., et al. (2014). Perivascular leukocyte clusters are essential for efficient activation of effector T cells in the skin. Nat. Immunol. 15, 1064-1069.
Núñez, N. G., Tosello Boari, J., Ramos, R. N., Richer, W., Cagnard, N., Anderfuhren, C. D., Niborski, L. L., Bigot, J., Meseure, D., De La Rochere, P., et al. (2020). Tumor invasion in draining lymph nodes is associated with Treg accumulation in breast cancer patients. Nature Communications 11, 3272.
Pagès, F., Mlecnik, B., Marliot, F., Bindea, G., Ou, F.-S., Bifulco, C., Lugli, A., Zlobec, I., Rau, T. T., Berger, M. D., et al. (2018). International validation of the consensus Immunoscore for the classification of colon cancer: a prognostic and accuracy study. The Lancet 397, 2128-2139.
Peranzoni, E., Lemoine, J., Vimeux, L., Feuillet, V., Barrin, S., Kantari-Mimoun, C., Bercovici, N., Guérin, M., Biton, J., Ouakrim, H., et al. (2018). Macrophages impede CD8 T cells from reaching tumor cells and limit the efficacy of anti-PD-1 treatment. PNAS 115, E4041-E4050.
Pucci, F., Venneri, M. A., Biziato, D., Nonis, A., Moi, D., Sica, A., Di Serio, C., Naldini, L., and De Palma, M. (2009). A distinguishing gene signature shared by tumor-infiltrating Tie2-expressing monocytes, blood “resident” monocytes, and embryonic macrophages suggests common functions and developmental relationships. Blood 114, 901-914.
Qian, B.-Z., Li, J., Zhang, H., Kitamura, T., Zhang, J., Campion, L. R., Kaiser, E. A., Snyder, L. A., and Pollard, J. W. (2011). CCL2 recruits inflammatory monocytes to facilitate breast-tumour metastasis. Nature 475, 222-225.
Ramos, R. N., Rodriguez, C., Hubert, M., Ardin, M., Treilleux, I., Ries, C.H., Lavergne, E., Chabaud, S., Colombe, A., Trédan, O., et al. (2020). CD163+ tumor-associated macrophage accumulation in breast cancer patients reflects both local differentiation signals and systemic skewing of monocytes. Clin Transl Immunology 9, e1108.
Ries, C. H., Cannarile, M. A., Hoves, S., Benz, J., Wartha, K., Runza, V., Rey-Giraud, F., Pradel, L.P., Feuerhake, F., Klaman, I., et al. (2014). Targeting tumor-associated macrophages with anti-CSF-1R antibody reveals a strategy for cancer therapy. Cancer Cell 25, 846-859.
Ruffell, B., and Coussens, L. M. (2015). Macrophages and Therapeutic Resistance in Cancer. Cancer Cell 27, 462-472.
Ruffell, B., Au, A., Rugo, H. S., Esserman, L. J., Hwang, E. S., and Coussens, L. M. (2012). Leukocyte composition of human breast cancer. PNAS 109, 2796-2801.
Samaniego, R., Palacios, B. S., Domiguez-Soto, Á., Vidal, C., Salas, A., Matsuyama, T., Sánchez-Torres, C., Torre, I. de la, Miranda-Carús, M. E., Sánchez-Mateos, P., et al. (2014). Macrophage uptake and accumulation of folates are polarization-dependent in vitro and in vivo and are regulated by activin A. Journal of Leukocyte Biology 95, 797-808.
Sg. U., P. S., W. M., Ms, T., A. S., M, L., F. I., Na, R., I, L., A, B., et al. (2020). Early Fate Defines Microglia and Non-parenchymal Brain Macrophage Development (Cell).
Sharma, A., Seow, Justine Jia Wen, and Dutertre, Charles-Antoine (2020). Onco-fetal Reprogramming of Endothelial Cells Drives Immunosuppressive Macrophages in Hepatocellular Carcinoma. Cell 183, 40.
Stuart, T., Butler, A., Hoffman, P., Hafemeister, C., Papalexi, E., Mauck, W. M., Hao, Y., Stoeckius, M., Smibert, P., and Satija, R. (2019). Comprehensive Integration of Single-Cell Data. Cell 177, 1888- 1902.e21.
Thomas, J. R., Appios, A., Zhao, X., Dutkiewicz, R., Donde, M., Lee, C. Y. C., Naidu, P., Lee, C., Cerveira, J., Liu, B., et al. (2021). Phenotypic and functional characterization of first-trimester human placental macrophages, Hofbauer cells. J Exp Med 218.
Van Nguyen, A., and Pollard, J. W. (2002). Colony Stimulating Factor-1 Is Required to Recruit Macrophages into the Mammary Gland to Facilitate Mammary Ductal Outgrowth. Developmental Biology 247, 11-25.
Veglia, F., Perego, M., and Gabrilovich, D. (2018). Myeloid-derived suppressor cells coming of age. Nature Immunology 19, 108-119.
Villani, A.-C., Satija, R., Reynolds, G., Sarkizova, S., Shekhar, K., Fletcher, J., Griesbeck, M., Butler, A., Zheng, S., Lazo, S., et al. (2017). Single-cell RNA-seq reveals new types of human blood dendritic cells, monocytes, and progenitors. Science 356, eaah4573.
Wang, Y., Chaffee, T.S., LaRue, R.S., Huggins, D. N., Witschen, P. M., Ibrahim, A. M., Nelson, A. C., Machado, H.L., and Schwertfeger, K. L. (2020). Tissue-resident macrophages promote extracellular matrix homeostasis in the mammary gland stroma of nulliparous mice. ELife 9, e57438.
Yuan, Z.-Y., Luo, R.-Z., Peng, R.-J., Wang, S.-S., and Xue, C. (2014). High infiltration of tumor-associated macrophages in triple-negative breast cancer is associated with a higher risk of distant metastasis (Dove Press).
Zhang, Q., He, Y., Luo, N., Patel, S.J., Han, Y., Gao, R., Modak, M., Carotta, S., Haslinger, C., Kind, D., et al. (2019). Landscape and Dynamics of Single Immune Cells in Hepatocellular Carcinoma. Cell 179, 829-845.e20.
Zhao, X., Qu, J., Sun, Y., Wang, J., Liu, X., Wang, F., Zhang, H., Wang, W., Ma, X., Gao, X., et al. (2017). Prognostic significance of tumor-associated macrophages in breast cancer: a meta-analysis of the literature. Oncotarget 8.
Zhu, Y., Herndon, J. M., Sojka, D. K., Kim, K.-W., Knolhoff, B. L., Zuo, C., Cullinan, D. R., Luo, J., Bearden, A. R., Lavine, K. J., et al. (2017). Tissue-Resident Macrophages in Pancreatic Ductal Adenocarcinoma Originate from Embryonic Hematopoiesis and Promote Tumor Progression. Immunity 47, 323-338.e6.

1-16. (canceled)

17. A method of treating cancer in a patient, comprising:
determining the level of FOLR2+ macrophages in a patient tumor sample, wherein the level of tumor-associated FOLR2+ macrophages correlates positively with outcome of cancer disease or treatment in the patient;
deducing therefrom whether the outcome of cancer disease or treatment is likely to be favorable or unfavorable in the patient; and
administering an appropriate treatment to the patient depending on whether the outcome of cancer disease or treatment is likely to be favorable or not in the patient.

18. The method according to claim 17, wherein an elevated level of FOLR2+ macrophages in the patient tumor sample as compared to a reference, indicates that the outcome of cancer disease or treatment is likely to be favorable in the patient.

19. The method according to claim 17, wherein the favorable outcome of cancer disease comprises an increased survival time or rate, a decreased rate of relapse, an increased time to relapse, and/or a reduced tumor evolution or metastasis.

20. The method according to claim 17, which comprises determining the density of FOLR2+ cells in the patient tumor sample.

21. The method according to claim 20, comprising determining the density of FOLR2+ cells in the patient tumor sample by immunohistochemical technique using anti-FOLR2 antibody.

22. The method according to claim 20, wherein the FOLR2+ cells are further TREM2− or TREM2^{low} and/or CADM1−.

23. The method according to claim 17, which comprises determining the level of expression of FOLR2 gene in the patient tumor sample.

24. The method according to claim 23, which comprises determining the level of FOLR2 protein in the patient tumor sample.

25. The method according to claim 17, which comprises determining the level of expression of a gene signature of tumor-associated FOLR2+ macrophages, which comprises or consists of the FOLR2, SEPP1 and SLC40A1 genes.

26. The method according to claim 25, which comprises determining the level(s) of mRNA expressed by the FOLR2 gene or the FOLR2, SEPP1 and SLC40A1 genes by RNA-Seq.

27. The method according to claim 17, further comprising a step of classification of the patient into favorable and unfavorable prognosis groups based on the level of tumor-associated FOLR2+ macrophages determined in the patient tumor sample.

28. The method according to claim 27, comprising the administration of an immune checkpoint blockage agent or an endocrine therapy agent, if the patient is classified as having a favorable prognosis.

29. The method according to claim 27, comprising the administration of a chemotherapy agent, or a combination of chemotherapy agent and immunotherapy agent or endocrine therapy agent if the patient is classified as having an unfavorable prognosis.

30. The method according to claim 17, wherein the cancer is selected from the group consisting of: breast, kidney, lung, liver, skin, uterus, brain, thyroid and adrenal gland cancer.

31. A targeted antigen delivery system comprising a FOLR2 binding ligand associated with an antigen of interest or a nucleic acid encoding the antigen in expressible form.

32. The targeted antigen delivery system according to claim 31, wherein the antigen of interest is a vaccine antigen.

33. The targeted antigen delivery system according to claim 31, wherein the FOLR2 binding ligand comprises an anti-FOLR2 antibody or fragment thereof comprising the antigen-binding site.

34. The targeted antigen delivery system according to claim 31, wherein the FOLR2 binding ligand and antigen or nucleic acid thereof are associated in a conjugate, a fusion protein or a particle.

35. A pharmaceutical composition, comprising the antigen delivery system according to claim 31, and at least one pharmaceutically acceptable vehicle, adjuvant and/or carrier.

36. A gene signature of tumor-associated FOLR2+ macrophages comprising or consisting of the FOLR2, SEPP1 and SLC40A1 genes.
